SEQUENCE LISTING (60 sequences)

SEQ ID NO: 1 - protein, 52 aa, Streptococcus zooepidemicus
DITGAALLEAKEAAINELKQYGISDYYVTLINKAKTVEGVNALKAEILSALP

SEQ ID NO: 2 - protein, 111 aa, Unknown
  Description of Unknown Rabbit polypeptide
ELVLTQTPASVEAAVGGTVTIKCQASQSISSYLAWYQQKPGHPPKLLIYKASTLASGVSSRFKGSGSGTEFTLTISDLECADAATYYCQSTYLGGTYVGGAFGGGTELEIL

SEQ ID NO: 3 - protein, 110 aa, Unknown
  Description of Unknown Rabbit polypeptide
ELVMTQTPASVEAAVGGTVTIKCQASQSISRYLAWYQQKPGQPPKLLIYRASTLASGVSSRFKGSGSGTEFTLTISDLECADAATYYCQSTYVNRGSVAAFGGGTEVVVK

SEQ ID NO: 4 - protein, 107 aa, Unknown
  Description of Unknown Rabbit polypeptide
ELVLTQTPSSVSAAVGGTVTINCQASESIGNYLAWYQQKPGQPPKLLLYSASTLESGVPSRFRGSGSGTEFTLTISGMKAEDAATYYCQSGYYSALTFGAGTNVEIK

SEQ ID NO: 5 - protein, 111 aa, Unknown
  Description of Unknown Rabbit polypeptide
ELDMTQTPASVSAAVGGTVTINCQASQNIYSNLAWYQQKPGQRPKLLIYAASNLASGVPSRFSGSGSGTDFTLTISDLECADAATYYCQSYYSSSSRYGNGFGGGTELEIL

SEQ ID NO: 6 - protein, 110 aa, Unknown
  Description of Unknown Rabbit polypeptide
ELVLTQTPASVSAAVGGTVTINCQASQNISSWLSWYQQKPGQPPKLLIYRASTLASGVPSRFKGSGSGTQFTLTISDLECDDAATYYCQQTYTYTNIENAFGGGTELEIL

SEQ ID NO: 7 - protein, 108 aa, Unknown
  Description of Unknown Rabbit polypeptide
ELVMTQTPASVSEPVGGTVTIKCQASQSISNLLAWYQQKPGQPPKLLIYKASTLASGVSSRFKGSGSGTEFTLTISGVQCDDAATYACQCTGYGSDWAFGGGTEVVVK

SEQ ID NO: 8 - protein, 108 aa, Unknown
  Description of Unknown Rabbit polypeptide
ELVLTQTPSSVSAAVGGTVTINCQASQSVSNLLAWYQQKPGQPPKLLIYGASNLESGVPSRFRGSGSGTEFTLTISGMKAEDAATYYCQSGYYSAGATFGAGTNVEIK

SEQ ID NO: 9 - protein, 107 aa, Unknown
  Description of Unknown Rabbit polypeptide
ELDLTQTPSSVSAAVGGTVTINCQASQSVSNLLAWYQQKPGQPPKLLIYGASNLESGVPSRFRGSGSGTEFTLTISGMKAEDAATYYCQSGYYSALTFGAGTNVEIK

SEQ ID NO: 10 - protein, 111 aa, Unknown
  Description of Unknown Rabbit polypeptide
ELVMTQTPSSVSAAVGGTVTINCQASPSVYKNYLAWFQQKPGQPPKLLIYLTSTLASGVPSRFKGSGSGTEFTLTISDVQCDDAATYYCLGGYGDIDDTHTFGGGTELEIL

SEQ ID NO: 11 - protein, 106 aa, Unknown
  Description of Unknown Rabbit polypeptide
ELVMTQTPSSVSAAVGGTVTIKCQASQSVNNYLAWYRHKPRQPPQLLIGASNLESGVASWFRGRGSGTGINLTITGMKAEDVAAYYCYRHSNCYLTFGAGTNVEIK

SEQ ID NO: 12 - protein, 109 aa, Unknown
  Description of Unknown Rabbit polypeptide
ELDLTQTPPSLSASVGETVRIRCLASEDIYSGISWYQQKPEKPPRLLISGASRLESGVPPRFSGSGSGTDYTLTIGGVQTEDAATYYCLGGSYGSTGMTFGAGTNVEIK

SEQ ID NO: 13 - protein, 109 aa, Unknown
  Description of Unknown Rabbit polypeptide
ELVMTQTPPSLSASVGETVRIRCLASEDIYSGISWYQQKPGKPPTLLIYGASNLESGVPPRFSGSGSGTDYTLTIGGVQAEDAATYYCLGGYTYSGTLTFGAGTNVEIK

SEQ ID NO: 14 - protein, 111 aa, Unknown
  Description of Unknown Rabbit polypeptide
ELVMTQTPASVSAAVGGTVTINCQASQSIGNNYLCWYQKKRGPPPMLLIYKASPLASGVPSRFEGGGSGTEYPLTIGGVRGDDAASYCCQGYIGYNNDDHAFGGGTEVVVK

SEQ ID NO: 15 - protein, 112 aa, Unknown
  Description of Unknown Rabbit polypeptide
ELVLTQTPASVEAAVGGTVTIKCQASESISNYLAWYQQKPGQPPKLLIYSASTLASGVPSRFSGSGSGTEFTLTISDLECADAATYYCQCTVAISGNSYGNAFGGGTEVVVK

SEQ ID NO: 16 - protein, 108 aa, Unknown
  Description of Unknown Rabbit polypeptide
ELDLTQTPSSVSAAVGGTVTINCQASQSVSNLLAWYQQKPGQPPKLLIYGASNLESGVPSRFRGSGSGTEFTLTISGMKAEDAATYYCQSGYYSAGATFGAGTNVEIK

SEQ ID NO: 17 - protein, 107 aa, Unknown
  Description of Unknown Rabbit polypeptide
ELVMTQTPASVSEPVGGTVTIKCQASQSIYSYLSWYQQKPGQRPKLLIYEASKLASGVPSRFSGSGSGTEFTLTISDLECADAATYYCQYSGGSIYSFGGGTELEIL

SEQ ID NO: 18 - protein, 110 aa, Unknown
  Description of Unknown Rabbit polypeptide
ELDLTQTPASVEAAVGGTVTIKCQASQSISSYLAWYQQKPGQPPKLLIYRASTLASGVSSRFSGSGSGTEFTLTISDLECADAATYYCQCTYAGSSYGNTFGGGTEVVVK

SEQ ID NO: 19 - protein, 109 aa, Unknown
  Description of Unknown Rabbit polypeptide
ELDMTQTPSPVSGAVGGTVTIKCQASQNIIGYLSWYQQKPGQPPKLLIYRASTLASGVPSRFSGSGSGTQFTLTISDVQCDDAATYYCAGDYTDNSDNAFGGGTEVVVK

SEQ ID NO: 20 - protein, 111 aa, Unknown
  Description of Unknown Rabbit polypeptide
ELVLTQQPASVEAAPGGTVTIKCQASQSISSYLAWYQQKPGHPPKLLIYKVSTRASGVSSRFSGSGSGTEFTLTISDLKSADAATYYCQSAYLGGTYVGGAFGGGTELEIK

SEQ ID NO: 21 - protein, 111 aa, Unknown
  Description of Unknown Rabbit polypeptide
ELVLTQQPASVEAAPGGTVTIKCQASQSISSYLAWYQQKPGHPPKLLIYKVSTEASGVSSRFSGSGSGTEFTLTISDLKSADAATYYCQSAYLGGTYVGGAFGGGTELEIK

SEQ ID NO: 22 - protein, 111 aa, Unknown
  Description of Unknown Rabbit polypeptide
ELVLTQQPASVEAAPGGTVTIKCQASQSISSYLAWYQQKPGHPPKLLIYKVSTRASGVSSRFSGSGSGTEFTLTISDLKSADAATYYCQSTYLGGTYVGGAFGGGTELEIK

SEQ ID NO: 23 - protein, 111 aa, Unknown
  Description of Unknown Rabbit polypeptide
ELVLTQQPASVEAAPGGTVTIKCQASQSISSYLAWYQQKPGHPPKLLIYKVSTEASGVSSRFSGSGSGTEFTLTISDLKSADAATYYCQSTYLGGTYVGGAFGGGTELEIK

SEQ ID NO: 24 - protein, 111 aa, Unknown
  Description of Unknown Rabbit polypeptide
ELVLTQQPASVEAAPGGTVTIKCQASQSISSYLAWYQQKPGHPPKLLIYKASTLASGVSSRFSGSGSGTEFTLTISDLKSADAATYYCQSTYLGGTYVGGAFGGGTELEIK

SEQ ID NO: 25 - protein, 110 aa, Unknown
  Description of Unknown Rabbit polypeptide
ELVMTQQPASVEAAPGGTVTIKCQASQSISSYLAWYQQKPGQPPKLLIYRVSTRASGVSSRFSGSGSGTEFTLTISDLKSADAATYYCQSAYVNRGSVASFGGGTKVVVK

SEQ ID NO: 26 - protein, 110 aa, Unknown
  Description of Unknown Rabbit polypeptide
ELVMTQQPASVEAAPGGTVTIKCQASQSISSYLAWYQQKPGQPPKLLIYRVSTEASGVSSRFSGSGSGTEFTLTISDLKSADAATYYCQSAYVNRGSVASFGGGTKVVVK

SEQ ID NO: 27 - protein, 110 aa, Unknown
  Description of Unknown Rabbit polypeptide
ELVMTQQPASVEAAPGGTVTIKCQASQSISSYLAWYQQKPGQPPKLLIYRVSTRASGVSSRFSGSGSGTEFTLTISDLKSADAATYYCQSTYVNRGSVAAFGGGTKVVVK

SEQ ID NO: 28 - protein, 110 aa, Unknown
  Description of Unknown Rabbit polypeptide
ELVMTQQPASVEAAPGGTVTIKCQASQSISSYLAWYQQKPGQPPKLLIYRVSTEASGVSSRFSGSGSGTEFTLTISDLKSADAATYYCQSTYVNRGSVAAFGGGTKVVVK

SEQ ID NO: 29 - protein, 110 aa, Unknown
  Description of Unknown Rabbit polypeptide
ELVMTQQPASVEAAPGGTVTIKCQASQSISSYLAWYQQKPGQPPKLLIYRASTLASGVSSRFSGSGSGTEFTLTISDLKSADAATYYCQSTYVNRGSVAAFGGGTKVVVK

SEQ ID NO: 30 - protein, 18 aa, Artificial Sequence
  Description of Artificial Sequence Synthetic peptide
SGGGGSGGGGSGGGGSSG

SEQ ID NO: 31 - protein, 52 aa, Streptococcus zooepidemicus
DITGAALLEAKDAAINELKQYGISDYYVHLINKADTVEGVNALKDEILSALP

SEQ ID NO: 32 - protein, 239 aa, Artificial Sequence
  Description of Artificial Sequence Synthetic polypeptide
ELVLTQTPASVEAAVGGTVTIKCQASQSISSYLAWYQQKPGHPPKLLIYKASTLASGVSSRFKGSGSGTEFTLTISDLECADAATYYCQSTYLGGTYVGGAFGGGTELEILSGGGGSGGGGSGGGGSSGELVMTQTPASVEAAVGGTVTIKCQASQSISRYLAWYQQKPGQPPKLLIYRASTLASGVSSRFKGSGSGTEFTLTISDLECADAATYYCQSTYVNRGSVAAFGGGTEVVVK

SEQ ID NO: 33 - protein, 309 aa, Artificial Sequence
  Description of Artificial Sequence Synthetic polypeptide
ELVLTQTPASVEAAVGGTVTIKCQASQSISSYLAWYQQKPGHPPKLLIYKASTLASGVSSRFKGSGSGTEFTLTISDLECADAATYYCQSTYLGGTYVGGAFGGGTELEILSGGGGSGGGGSGGGGSSGELVMTQTPASVEAAVGGTVTIKCQASQSISRYLAWYQQKPGQPPKLLIYRASTLASGVSSRFKGSGSGTEFTLTISDLECADAATYYCQSTYVNRGSVAAFGGGTEVVVKSGGGGSGGGGSGGGGSSGDITGAALLEAKDAAINELKQYGISDYYVHLINKADTVEGVNALKDEILSALP

SEQ ID NO: 34 - protein, 309 aa, Artificial Sequence; identical to SEQ ID NO: 33
  Description of Artificial Sequence Synthetic polypeptide

SEQ ID NO: 35 - protein, 239 aa, Artificial Sequence
  Description of Artificial Sequence Synthetic polypeptide
ELVLTQQPASVEAAPGGTVTIKCQASQSISSYLAWYQQKPGHPPKLLIYKVSTRASGVSSRFSGSGSGTEFTLTISDLKSADAATYYCQSAYLGGTYVGGAFGGGTELEIKSGGGGSGGGGSGGGGSSGELVMTQQPASVEAAPGGTVTIKCQASQSISSYLAWYQQKPGQPPKLLIYRVSTRASGVSSRFSGSGSGTEFTLTISDLKSADAATYYCQSAYVNRGSVASFGGGTKVVVK

SEQ ID NO: 36 - protein, 239 aa, Artificial Sequence
  Description of Artificial Sequence Synthetic polypeptide
ELVLTQQPASVEAAPGGTVTIKCQASQSISSYLAWYQQKPGHPPKLLIYKVSTEASGVSSRFSGSGSGTEFTLTISDLKSADAATYYCQSAYLGGTYVGGAFGGGTELEIKSGGGGSGGGGSGGGGSSGELVMTQQPASVEAAPGGTVTIKCQASQSISSYLAWYQQKPGQPPKLLIYRVSTEASGVSSRFSGSGSGTEFTLTISDLKSADAATYYCQSAYVNRGSVASFGGGTKVVVK

SEQ ID NO: 37 - protein, 239 aa, Artificial Sequence
  Description of Artificial Sequence Synthetic polypeptide
ELVLTQQPASVEAAPGGTVTIKCQASQSISSYLAWYQQKPGHPPKLLIYKVSTRASGVSSRFSGSGSGTEFTLTISDLKSADAATYYCQSTYLGGTYVGGAFGGGTELEIKSGGGGSGGGGSGGGGSSGELVMTQQPASVEAAPGGTVTIKCQASQSISSYLAWYQQKPGQPPKLLIYRVSTRASGVSSRFSGSGSGTEFTLTISDLKSADAATYYCQSTYVNRGSVAAFGGGTKVVVK

SEQ ID NO: 38 - protein, 239 aa, Artificial Sequence
  Description of Artificial Sequence Synthetic polypeptide
ELVLTQQPASVEAAPGGTVTIKCQASQSISSYLAWYQQKPGHPPKLLIYKVSTEASGVSSRFSGSGSGTEFTLTISDLKSADAATYYCQSTYLGGTYVGGAFGGGTELEIKSGGGGSGGGGSGGGGSSGELVMTQQPASVEAAPGGTVTIKCQASQSISSYLAWYQQKPGQPPKLLIYRVSTEASGVSSRFSGSGSGTEFTLTISDLKSADAATYYCQSTYVNRGSVAAFGGGTKVVVK

SEQ ID NO: 39 - protein, 239 aa, Artificial Sequence
  Description of Artificial Sequence Synthetic polypeptide
ELVLTQQPASVEAAPGGTVTIKCQASQSISSYLAWYQQKPGHPPKLLIYKASTLASGVSSRFSGSGSGTEFTLTISDLKSADAATYYCQSTYLGGTYVGGAFGGGTELEIKSGGGGSGGGGSGGGGSSGELVMTQQPASVEAAPGGTVTIKCQASQSISSYLAWYQQKPGQPPKLLIYRASTLASGVSSRFSGSGSGTEFTLTISDLKSADAATYYCQSTYVNRGSVAAFGGGTKVVVK

SEQ ID NO: 40 - protein, 309 aa, Artificial Sequence
  Description of Artificial Sequence Synthetic polypeptide
ELVLTQQPASVEAAPGGTVTIKCQASQSISSYLAWYQQKPGHPPKLLIYKVSTRASGVSSRFSGSGSGTEFTLTISDLKSADAATYYCQSAYLGGTYVGGAFGGGTELEIKSGGGGSGGGGSGGGGSSGELVMTQQPASVEAAPGGTVTIKCQASQSISSYLAWYQQKPGQPPKLLIYRVSTRASGVSSRFSGSGSGTEFTLTISDLKSADAATYYCQSAYVNRGSVASFGGGTKVVVKSGGGGSGGGGSGGGGSSGDITGAALLEAKDAAINELKQYGISDYYVHLINKADTVEGVNALKDEILSALP

SEQ ID NO: 41 - protein, 309 aa, Artificial Sequence
  Description of Artificial Sequence Synthetic polypeptide
ELVLTQQPASVEAAPGGTVTIKCQASQSISSYLAWYQQKPGHPPKLLIYKVSTEASGVSSRFSGSGSGTEFTLTISDLKSADAATYYCQSAYLGGTYVGGAFGGGTELEIKSGGGGSGGGGSGGGGSSGELVMTQQPASVEAAPGGTVTIKCQASQSISSYLAWYQQKPGQPPKLLIYRVSTEASGVSSRFSGSGSGTEFTLTISDLKSADAATYYCQSAYVNRGSVASFGGGTKVVVKSGGGGSGGGGSGGGGSSGDITGAALLEAKDAAINELKQYGISDYYVHLINKADTVEGVNALKDEILSALP

SEQ ID NO: 42 - protein, 309 aa, Artificial Sequence
  Description of Artificial Sequence Synthetic polypeptide
ELVLTQQPASVEAAPGGTVTIKCQASQSISSYLAWYQQKPGHPPKLLIYKVSTRASGVSSRFSGSGSGTEFTLTISDLKSADAATYYCQSTYLGGTYVGGAFGGGTELEIKSGGGGSGGGGSGGGGSSGELVMTQQPASVEAAPGGTVTIKCQASQSISSYLAWYQQKPGQPPKLLIYRVSTRASGVSSRFSGSGSGTEFTLTISDLKSADAATYYCQSTYVNRGSVAAFGGGTKVVVKSGGGGSGGGGSGGGGSSGDITGAALLEAKDAAINELKQYGISDYYVHLINKADTVEGVNALKDEILSALP

SEQ ID NO: 43 - protein, 309 aa, Artificial Sequence
  Description of Artificial Sequence Synthetic polypeptide
ELVLTQQPASVEAAPGGTVTIKCQASQSISSYLAWYQQKPGHPPKLLIYKVSTEASGVSSRFSGSGSGTEFTLTISDLKSADAATYYCQSTYLGGTYVGGAFGGGTELEIKSGGGGSGGGGSGGGGSSGELVMTQQPASVEAAPGGTVTIKCQASQSISSYLAWYQQKPGQPPKLLIYRVSTEASGVSSRFSGSGSGTEFTLTISDLKSADAATYYCQSTYVNRGSVAAFGGGTKVVVKSGGGGSGGGGSGGGGSSGDITGAALLEAKDAAINELKQYGISDYYVHLINKADTVEGVNALKDEILSALP

SEQ ID NO: 44 - protein, 309 aa, Artificial Sequence
  Description of Artificial Sequence Synthetic polypeptide
ELVLTQQPASVEAAPGGTVTIKCQASQSISSYLAWYQQKPGHPPKLLIYKASTLASGVSSRFSGSGSGTEFTLTISDLKSADAATYYCQSTYLGGTYVGGAFGGGTELEIKSGGGGSGGGGSGGGGSSGELVMTQQPASVEAAPGGTVTIKCQASQSISSYLAWYQQKPGQPPKLLIYRASTLASGVSSRFSGSGSGTEFTLTISDLKSADAATYYCQSTYVNRGSVAAFGGGTKVVVKSGGGGSGGGGSGGGGSSGDITGAALLEAKDAAINELKQYGISDYYVHLINKADTVEGVNALKDEILSALP

SEQ ID NO: 45 - protein, 5 aa, Artificial Sequence
  Description of Artificial Sequence Synthetic peptide
GGGGS

SEQ ID NO: 46 - protein, 4 aa, Artificial Sequence
  Description of Artificial Sequence Synthetic peptide
GGSG

SEQ ID NO: 47 - protein, 6 aa, Artificial Sequence
  Description of Artificial Sequence Synthetic peptide
GGSGGS

SEQ ID NO: 48 - protein, 9 aa, Artificial Sequence
  Description of Artificial Sequence Synthetic peptide
GGGGSGGGS

SEQ ID NO: 49 - protein, 10 aa, Artificial Sequence
  Description of Artificial Sequence Synthetic peptide
GGGGSGGGGS

SEQ ID NO: 50 - protein, 15 aa, Artificial Sequence
  Description of Artificial Sequence Synthetic peptide
GGGGSGGGGSGGGGS

SEQ ID NO: 51 - protein, 20 aa, Artificial Sequence
  Description of Artificial Sequence Synthetic peptide
GGGGSGGGGSGGGGSGGGGS

SEQ ID NO: 52 - protein, 25 aa, Artificial Sequence
  Description of Artificial Sequence Synthetic peptide
GGGGSGGGGSGGGGSGGGGSGGGGS

SEQ ID NO: 53 - protein, 30 aa, Artificial Sequence
  Description of Artificial Sequence Synthetic polypeptide
GGGGSGGGGSGGGGSGGGGSGGGGSGGGGS

SEQ ID NO: 54 - protein, 8 aa, Artificial Sequence
  Description of Artificial Sequence Synthetic peptide
SGGGGSSG

SEQ ID NO: 55 - protein, 13 aa, Artificial Sequence
  Description of Artificial Sequence Synthetic peptide
SGGGGSGGGGSSG

SEQ ID NO: 56 - protein, 23 aa, Artificial Sequence
  Description of Artificial Sequence Synthetic peptide
SGGGGSGGGGSGGGGSGGGGSSG

SEQ ID NO: 57 - protein, 28 aa, Artificial Sequence
  Description of Artificial Sequence Synthetic peptide
SGGGGSGGGGSGGGGSGGGGSGGGGSSG

SEQ ID NO: 58 - protein, 38 aa, Artificial Sequence
  Description of Artificial Sequence Synthetic polypeptide
SGGGGSGGGGSGGGGSGGGGSGGGGSGGGGSGGGGSSG

SEQ ID NO: 59 - protein, 21 aa, Artificial Sequence
  Description of Artificial Sequence Synthetic peptide
PTPTPTTPTPTPTTPTPTPTS

SEQ ID NO: 60 - protein, 9 aa, Artificial Sequence
  Description of Artificial Sequence Synthetic peptide
YPYDVPDYA

(19) United States
(12) Patent Application Publication
(10) Pub. No.: US 2015/0044208 A1
(43) Pub. Date: Feb. 12, 2015

(54) Modified Albumin-Binding Domains and Uses Thereof to Improve Pharmacokinetics

(71) Applicant: TECHNOPHAGE, INVESTIGAÇÄO E DESENVOLVIMENTO EM BIOTECNOLOGIA, SA, Lisboa (PT)

(72) Inventors: Frederico Nuno Castanheira Aires da Silva, Lisboa (PT); Sofia Volker Côrte-Real, Cruz Quebrada (PT); Sara Ferreira Llorente Grancho Lourenço, Massama (PT)

(73) Assignee: TECHNOPHAGE, INVESTIGAÇÄO E DESENVOLVIMENTO EM BIOTECNOLOGIA, SA, Lisboa (PT)

(21) Appl. No.: 14/346,126

(22) PCT Filed: Sep. 19, 2012
(86) PCT No.: PCT/PT2012/000036

§ 371 Date: Mar. 20, 2014

Related U.S. Application Data
(60) Provisional application No. 61/538,552, filed on Sep. 23, 2011.

(51) Int. Cl. C07K 14/315; C07K 16/24

(52) U.S. Cl. CPC C07K 14/315; C07K 16/241; C07K 2317/94; C07K 2319/70
USPC 424/134.1; 530/387.3; 530/391.3; 536/23.4; 435/69.6

(57) ABSTRACT

The present invention relates to compositions and methods comprising a modified albumin-binding domain to improve the pharmacokinetic properties of therapeutic molecules. The modified peptides show reduced immunogenicity and/or proved solubility. In particular, compositions and methods for enhancing therapeutic potential of protein therapeutics are provided including linking a protein albumin-binding domain, which has been modified to reduce immunogenicity and/or improve solubility, to a therapeutic protein, including therapeutic antibodies, antibody fragments, antibody single domains and/or dimers of antibody single domains. These linked polypeptides can exhibit enhanced serum half life without exacerbated immunogenicity and/or without decreased solubility, and without substantially affecting the specific binding properties of the therapeutic protein.

### CROSS-REFERENCE TO RELATED APPLICATIONS

This is application claims the benefit of U.S. Provisional Application Ser. No. 61/538,552, filed Sep. 23, 2011, the contents of which are hereby incorporated-by-reference in its entirety. This application also relates to U.S. Provisional Application Ser. No. 61/538,548, entitled “Anti-Tumor Necorsis Factor-alpha Agents and Uses Thereof,” filed by Frederico Aires da Silva, Sofia Volker Côrte-Real, and Sara Lourenco also on Sep. 23, 2011, and the contents of which also are hereby incorporated-by-reference in its entirety.

### FIELD OF THE INVENTION

The present invention relates to compositions and methods comprising a modified albumin-binding domain to improve the pharmacokinetic properties of therapeutic molecules. The modified peptides show reduced immunogenicity and/or improved solubility. In particular, compositions and methods for enhancing therapeutic potential of protein therapeutics are provided including linking a protein albumin-binding domain, which has been modified to reduce immunogenicity and/or improve solubility, to a therapeutic protein, including therapeutic antibodies, antibody fragments, antibody single domains and/or dimers of antibody single domains. These linked polypeptides can exhibit enhanced serum half life without exacerbated immunogenicity and/or without decreased solubility, and without substantially affecting the specific binding properties of the therapeutic protein.

### BACKGROUND

The effectiveness of pharmaceuticals depends heavily on the intrinsic pharmacokinetics of the compounds. This is true of both small molecule drugs and therapeutic protein pharmaceuticals. Small molecule drugs have long relied on their association with various plasma components to improve their pharmacokinetic properties in vivo; however, although its half-life is extended, a drug associated with plasma protein is usually unavailable for binding to the target. Since only the unbound fraction of the small molecule is generally functionally active, a fine balance must be maintained between the concentration of free drug required for efficacy and the frequency at which it must be administered (Rowland, M. (ed) (1988) Clinical Pharmacokinetics: Concepts and Applications, 2nd Ed., Lea & Febiger, Philadelphia, Pa.).

Therapeutic proteins, including interferons, growth hormones, antibody fragments, and the like, generally possess short serum half-lives largely due to their small size. This makes the proteins highly susceptible to rapid renal clearance and to degradation by serum proteases. Because the kidney generally filters out molecules below 60 kDa, efforts to reduce clearance of therapeutic proteins have focused on increasing molecular size through chemical modifications such as glycosylation or the addition of polyethylene glycol polymers (i.e. PEG). Kurtzhals, P., et al. (1995) Biochem. J. 312,725-731; Markussen, J., et al. (1996) Diabetologia 39,281-288. Although chemical derivatization approaches have enhanced serum half life, the addition of these molecules may also decrease bioactivity and therapeutic efficacy, as well as increase immunogenicity of the constructs.

The use of immunoglobulins as therapeutic agents in particular has increased dramatically in recent years and has expanded to different areas of medical treatments. Such uses include treatment of agammaglobulinemia and hypogammaglobulinemia, as immunosuppressive agents for treating autoimmune diseases and graft-vs.-host (GVH) diseases, the treatment and management of inflammatory diseases like rheumatoid arthritis, the treatment of lymphoid malignancies, and passive immunotherapies for the treatment of various systemic and infectious diseases. Also, immunoglobulins are useful as in vivo diagnostic tools, for example, in diagnostic imaging procedures. However, a persisting issue in these therapies is the clearance of immunoglobulins from the circulation. The rate of immunoglobulin clearance directly affects the amount and frequency of dosage of the immunoglobulin and increased dosage and frequency may cause adverse effects in the patient, as well as increasing medical costs.

The use of antibody fragments as therapeutic proteins also has greatly increased in recent years. For example, antibody fragments such as sdAbs, scFv, diabodies and Fabs offer rapid tumor penetration and have been explored for these indications; however, these fragments also are cleared rapidly and their ability to be retained in the tumor is limited (Kashmiri, S. V. S. (2001) J. Nucl. Med. 42:1528-1529; Wu, A. M. and Yazaki, P. J. (2000) Q. J. Nucl. Med. 44:268-83).

Several strategies have been used to overcome the problems associated with short serum half-life. For example, certain protein fusions have been attempted to increase the size and thus stability and half-life of protein therapeutics. (Syed et al. (1997) Blood 89:3243-3252; Yeh, et al. (1992) Proc. Natl. Acad. Sci. U.S.A. 89:1904-1908). These have included fusions of therapeutic proteins to the Fc portion of an antibody IgG and to arabinogalactan-protein (AGP). For example, an AGP chimera with interferon α2 showed up to a 13-fold increased in vivo serum half-life while the biological activity remained similar to native interferon a2. See also, U.S. Patent Publication No. 2007/0050855 that provides certain fusion proteins of therapeutic proteins with soluble toxin receptor fragments and modified transferrin molecules to improve serum half life and stability.

Fusions to antibody fragments also have been used, e.g., in products for treating rheumatoid arthritis, including Etanercept (Enbrel) and Certolizumab pegol (Cimzia), as well as Remicade™ (Inflixmab), Humira (adalimumab), and Simponi (golimumab). Enbrel is a genetic fusion of a TNF-alpha receptor fused with the Fc portion of an antibody while Cimzia is a PEGylated Fab fragment. Nonetheless, many patients develop immunogenic responses towards the products, requiring switching to other treatments.

Fusions of therapeutic proteins to albumin have also been attempted. Albumin (molecular mass of 67 kDa) is the most abundant protein in plasma, present at 50 mg/ml, and having a half-life of 19 days in humans (Peters, T., Jr. (1985) Adv. Protein Chem. 37:161-245; Peters, T., Jr. (1996) All about Albumin, Academic Press, Inc., San Diego, Calif.). Albumin plays a vital role in vivo by reversibly binding and transporting a wide variety of endogenous substances as well as drugs, and several major small molecule binding sites in albumin have been described. (e.g., see, Frick, et al. (1994) Mol Microbiol. 12:143-51; and Akesson et al. (1994) Biochem. J 300:877-886). Direct fusion with albumin, however, has proved less than successful. For example, albumin fusion to antibodes have failed because the albumin can become denatured either by the coupling procedure or by the conjugation. This can result in high immunogenicity and in considerable uptake of the conjugates by the reticuloendothelial system, such that these conjugates are removed rapidly from the circulation and taken up in large amounts by the liver, rather than the target tissues. Stehle, G., et al. (1997) Anticancer Drugs 8:677; Stehle, G., et al. (1997) Anticancer Drugs 8:835; Kashmiri,S. V. S. (2001) J. Nucl. Med., 42, 1528-1529; Wu, A. M. et al. (2000) Q. J. Nucl. Med., 44, 268-83. See also, e.g., WO 07/146038 to Human Genome Sciences providing albumin fusion proteins to increase serum availability and shelf life stability of therapeutic proteins.

Still a further strategy involves coupling the therapeutic to another protein that will allow in vivo association to serum albumins. Examples of this approach have been described e.g. in EP 0486525 and U.S. Pat. No. 6,267,964, which describe the use of albumin-binding peptides or proteins derived from streptococcal protein G (SpG) for increasing the half-life of other proteins by fusing the bacterially-derived albumin-binding peptide to a therapeutic protein. The fusion protein is intended to bind serum albumin and thus enhance the half-life. For example, in one case, a recombinant fusion of the albumin-binding domain from streptococcal protein G to human complement receptor type I increased its half-life 3-fold to 5 h in rats (Makrides, S. et al. (1996) J Pharmacol. Exp. Ther. 277, 534-542); in another case, fusion to this domain enhanced the immunological response directed to peptide antigens (Sjolander, A et al. (1997) J Immunol. Methods 201, 115-123). Additional albumin-binding protein sequences are provided in, e.g., PCT Publication No. WO 91/19741, PCT Publication No. WO 05/097202, PCT Publication No. WO 01/45746 and U.S. Patent Publication No. 2004/0001827. Fusion products, however, are prone to misfolding and the fused region can create highly immunogenic sites, resulting in a strong immunological reaction to the construct upon administration to a subject. The fusion molecule also may be less soluble than the therapeutic molecule alone.

Accordingly, there remains a need in the art for strategies of improving the bioavailability, stability, and/or serum half-life of molecules to allow for more effective therapeutics, without exacerbating immunogenicity of the therapeutic in the subject and/or without excessively reducing solubility. The instant invention provides compositions and techniques directed to addresing these and other needs.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

The present invention provides compounds, compositions, and methods of using modified albumin-binding domains, that are de-immunized and/or modified to improve solubility. Specifically, de-immunized albumin-binding domains of Streptococcus zooepidemicus, as well as albumin-binding fragments or derivatives thereof, are provided, in which one or more T_{H} epitopes of the albumin-binding domain is reduced or eliminated to decrease immunogenicity of the peptide. When linked to a therapeutic molecule, the modified albumin-binding domains can enhance the pharmaceutical potential of the molecule, by increasing half-life but not exacerbating immunogenicity and/or not excessively decreasing solubility, while maintaining the therapeutic properties of the molecule.

Accordingly, one aspect of the invention relates to an agent comprising a therapeutic molecule linked to at least one albumin-binding domain, or an albumin-binding fragment or derivative thereof, where the albumin-binding domain comprises an amino acid sequence corresponding to SEQ ID NO:1 (PEP) and where the domain is modified by at least one amino acid substitution selected from the group consisting of E12D, T29H-K35D, and A45D, the substitutions referring to amino acid positions in SEQ ID NO:1; such that the agent has an increased serum half life as compared to the therapeutic molecule.

In some particular embodiments, the albumin-binding domain comprises the amino acid substitution E12D, the substitution referring to an amino acid position in SEQ ID NO:1. In some particular embodiments, the albumin-binding domain comprises the amino acid substitution T29H-K35D, the substitution referring to an amino acid position in SEQ ID NO:1. In some particular embodiments, the albumin-binding domain comprises the amino acid substitution A45D, the substitution referring to an amino acid position in SEQ ID NO:1. In some particular embodiments, the albumin-binding domain comprises an amino acid sequence corresponding to SEQ ID NO:31.

In some preferred embodiments, the serum half life of the agent is increased by at least about 5 fold compared to the therapeutic molecule without the albumin-binding domain. In some preferred embodiments, serum half life is increased by at least about 8 fold or by at least about 10 fold. In some preferred embodiments, the serum half life of the agent is at least about 30 hours, or at least about 40 hours.

In some preferred embodiments, the agent also has increased solubility compared to the therapeutic molecule. For example, in some preferred embodiments, the solubility is increased by at least about 2 fold, by at least about 5 fold, by at least about 10 fold, or by at least about 15 fold.

In some particular embodiments, the linkage between the therapeutic molecule and albumin-binding domain is via a linker. In some preferred embodiments, the linker is a peptide linker, e.g., a peptide linker comprising an amino acid sequence corresponding to SEQ ID NO:30.

In some particularly preferred embodiments, the therapeutic molecule is a therapeutic polypeptide or peptide and in some further embodiments, the therapeutic polypeptide or peptide is linked to the albumin-binding domain as a fusion. The therapeutic molecule may be selected from the group consisting of protamine, gp60, gp30, gp18, protein A, a G protein, a protein transduction domain, toxins, cytotoxins, radionuclides, and macrocyclic chelators. In some particularly preferred embodiments, the therapeutic molecule is an antibody or antibody fragment, such as, e.g., an antibody or antibody fragment selected from the group consisting of a monoclonal antibody, multispecific antibody, humanized antibody, synthetic antibody, chimeric antibody, polyclonal antibody, single-chain Fv (scFv), single chain antibody, anti-idiotypic (anti-Id) antibody, diabody, minibody, nanobody, single domain antibody, Fab fragment, F(ab′) fragment, disulfide-linked bispecific Fv (sdFv), and intrabody.

In some particular embodiments, the therapeutic molecule comprises a dimer of two antibody single domains or antigen-binding fragments thereof, where the domains comprise light chain variable domains. In some preferred embodiments, the dimer binds TNF-alpha. In some preferred embodiments, the dimer comprises at least one light chain variable domain comprising an amino acid sequence corresponding to SEQ ID NO:2 (VL18), SEQ ID NO:3 (VL11), SEQ ID NOs:4-19, or a TNF-alpha-binding fragment or derivative thereof. In some embodiments, the dimer comprises two light chain variable domains comprising amino acid sequences corresponding to SEQ ID NO:2 (VL18) and SEQ ID NO:3 (VL11), or a TNF-alpha-binding fragment or derivative thereof for one or both sequences. In some embodiments, the dimer comprises at least one amino acid sequence selected from the group consisting of SEQ ID NO:32 (VL18-3L-VL11), SEQ ID NOs: 34, and a TNF-alpha-binding fragment or derivative thereof.

In some embodiments, at least one variable domain antagonizes binding of human TNF-alpha to a TNF-alpha receptor. In some more preferred embodiments, the at least one variable domain further cross-reacts with at least one other mammalian TNF-alpha, where the mammal is not a primate. In some even more preferred embodiments, the variable domain cross-reacts with TNF-alpha of at least two other mammals, the at least two other mammals being a rodent and a non-rodent species.

In some embodiments, the dimer is further de-immunized by eliminating at least one T_{H} epitope of in at least one of the variable domains. For example, in some particular embodiments, at least one variable domain comprises an amino acid sequence corresponding to SEQ ID NO:2 (VL18), which is also de-immunized by at least one amino acid substitution selected from the group consisting of T7Q, V15P, (A51V-L54R/A51V-L54E), K63 S, E79K, (C80S), T91A, and L111K, the substitutions referring to amino acid positions in SEQ ID NO:2. In some particular embodiments, at least one variable domain comprises an amino acid sequence corresponding to SEQ ID NO:3 (VL11), which is also de-immunized by at least one amino acid substitution selected from the group consisting of T7Q, V15P, R31S, (A51V-54R/A51V-L54E), K63S, E79K, (C80S), T91A, A100S, and E106K, the substitutions referring to amino acid positions in SEQ ID NO:3. In some preferred embodiments, the dimer comprises at least one light chain variable domain comprising an amino acid sequence corresponding to SEQ ID NOs:20-24, SEQ ID NOs: 25-29, or a TNF-alpha-binding fragment or derivative thereof. In some preferred embodiments, the dimer comprises at least one amino acid sequence selected from the group consisting of SEQ ID NOs: 35-44 (VL18-3L-VL11/PEP variants), and a TNF-alpha-binding fragment or derivative thereof.

Another aspect of the instant invention relates to a method of enhancing the efficacy of a therapeutic molecule in a subject, comprising: providing an agent comprising the therapeutic molecule linked at least one albumin-binding domain, or an albumin-binding fragment or derivative thereof, where the albumin-binding domain comprises an amino acid sequence corresponding to SEQ ID NO:1 (PEP) where the domain is modified by at least one amino acid substitution selected from the group consisting of E12D, T29H-K35D, and A45D, the substitutions referring to amino acid positions in SEQ ID NO:1; and administering the agent to the subject.

Yet another aspect of the invention relates to a pharmaceutical composition comprising the agent according to the invention, and/or or a nucleic acid comprising a nucleotide sequence encoding an agent, and a pharmaceutically acceptable carrier. Still yet another aspect of the invention relates to a nucleic acid comprising a nucleotide sequence encoding an agent according to the invention, as well as vectors and/or host cells and/or pharmaceutical compositions comprising same.

Still yet another aspect of the invention relates to a method of making an agent according to the invention, comprising (i) providing a host cell comprising a vector encoding the agent; (ii) culturing the cell under conditions allowing expression of the agent; and (iii) recovering the agent from the culture.

### BRIEF DESCRIPTION OF THE FIGURES

FIG. 1 illustrate proposed positions for substitutions (highlighted in gray) in an albumin binding domain in accordance with the invention, using Kabat and Ordinal numbering

FIGS. 2A-B illustrate proposed positions for substitutions (highlighted in gray) in two VL single domain antibodies (A-B) for fusion with an albumin-binding domain of the invention, using Kabat and Ordinal numbering; CDRs are indicated by x.

FIGS. 3A-B illustrate features of constructs containing modified albumin-binding domains in accordance with the invention. FIG. 3A illustrates the mode of binding between a VL-VL-PEP construct containing an albumin-binding domain of the invention and human albumin; FIG. 3B illustrates pharmacokinetics of VL18-3L-VL11 and VL18-3L-VL11-PEP tested by administration to Wistar female rats to determine the serum half-life thereof in vivo; data were normalized considering maximal concentration as that assayed 5 minutes after administration (% of 5 min value).

FIG. 4 illustrates results of Coomassie Blue SDS-PAGE expression analysis of a recombinant de-immunized fusion comprising an anti-TNF-alpha polypeptide (VL-VL dimer) with an albumin-binding domain of the invention (VL18-3L-VL11 DI3-PEP DI #8). The gel position of the expressed fusion is indicated by the arrow. Lanes 1-4 represent the following: Lane 1: See Blue 2 Plus Ladder, 10 μL; Lane 2: Pre-induction sample, total protein; Lane 3: overnight post-induction sample, total protein; and Lane 4: overnight post-induction sample, soluble protien.

FIGS. 5A-E illustrate downstream process development for de-immunized fusions comprising an anti-TNF-alpha polypeptide (VL-VL dimer) with an albumin-binding domain of the invention. FIG. 5A illustrates a schematic representation of downstream process development. FIGS. 5B-C illustrate results of Coomassie Blue SDS-PAGE expression analysis following Protein L Affinity purification of a de-immunized fusion of the invention (VL18-3L-VL11 DI3-PEP DI #8). FIG. 5D illustrates results of Coomassie Blue SDS-PAGE expression analysis following SP Sepharose cation exchange chromatography of a de-immunized fusion of the invention (VL18-3L-VL11 DI3-PEP DI #8). FIG. 5E illustrates results of Coomassie Blue SDS-PAGE expression analysis following size exclusion chromatography of a de-immunized fusion of the invention(VL18-3L-VL11 DI3-PEP DI #8).

FIG. 6 illustrates disease progression induced in an adjuvant-induced arthritis model in Wistar female rats

FIG. 7 illustrates therapeutic effect of de-immunized fusions comprising an anti-TNF-alpha polypeptide (VL-VL dimer) with an albumin-binding domain of the invention in an established rat adjuvant induced arthritis model (AIA).

FIGS. 8A-I illustrates therapeutic effect of de-immunized VL-VL dimers and de-immunized fusions of the invention in an established rat adjuvant-induced arthritis model (AIA) based on histological analyses.

FIGS. 9A-B illustrate biodistribution data for fac-[^{99m}Tc(CO)₃]-VL18-3L-VL11 (FIG. 9A) and fac-[^{99m}Tc(CO)₃]-VL18-3L-VL11-PEP (FIG. 9B) in relevant organs, expressed as % ID/Organ for 15 min, 1 h, 3 h, 6 h, and 24 h after i.p. administration in Wistar rats (n=3).

### DETAILED DESCRIPTION

### 1. Definitions

The term “derivative” when used in the context of a protein agent (including full length proteins, multimeric proteins, polypeptides, peptides, antibodies and fragments thereof, and specifically including domains such as albumin-binding domains) refers to an agent that possesses a similar or identical function as a second agent but does not necessarily comprise a similar or identical amino acid sequence, modifications such as glycosylation, or secondary, tertiary or quaternary structure of the second agent. A protein agent that has a similar amino acid sequence refers to a second protein agent that satisfies at least one of the following: (a) a protein agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a second protein agent; (b) a protein agent encoded by a nucleotide sequence that hybridizes under stringent conditions to a nucleotide sequence encoding a second protein agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a protein agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a second protein agent. A protein agent with similar structure to a second protein agent refers to a protein agent that has a similar secondary, tertiary or quaternary structure to the second protein agent. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403.

As used herein, the term “derivative” in the context of proteins, including albumin-binding domains and fragments thereof, also refers to a polypeptide or peptide that comprises an amino acid sequence which has been altered by the introduction of amino acid residue substitutions, deletions or additions. The term “derivative” as used herein also refers to a polypeptide or peptide which has been modified, i.e., by the covalent attachment of any type of molecule to the polypeptide or peptide. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative polypeptide or peptide may be produced by chemical modifications using techniques known to those of skill in the art, including, but not limited to specific chemical cleavage, acetylation, formylation, metabolic synthesis of tunicamycin, etc. Further, a derivative polypeptide or peptide derivative possesses a similar, identical, or improved function as the polypeptide or peptide from which it was derived.

As used herein, “derivative” in used interchangeably with “variant.”

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide. In a specific embodiment, a fragment of a polypeptide retains at least one function of the polypeptide.

As used herein, the terms “heavy chain,” “light chain,” “variable region,” “framework region,” “constant domain,” and the like, have their ordinary meaning in the immunology art and refer to domains in naturally occurring immunoglobulins and the corresponding domains of synthetic (e.g., recombinant) binding proteins (e.g., humanized antibodies, single chain antibodies, chimeric antibodies, etc.). The basic structural unit of naturally occurring immunoglobulins (e.g., IgG) is a tetramer having two light chains (L) and two heavy chains (H), usually expressed as a glycoprotein of about 150,000 Da. The amino-terminal (“n”) portion of each chain includes a variable region of about 100 to 110 or more amino acids primarily responsible for antigen recognition. The carboxy-terminal (“c”) portion of each chain defines a constant region, with light chains having a single constant domain and heavy chains usually having three constant domains and a hinge region. Accordingly, each light chain is made up generally of a variable domain (VL) and a constant domain (CL); while each heavy chain generally involves a variable domain (VH) and three constant domains (CH1, CH2, and CH3), as well a a hinge region (H). Thus, the structure of the light chains of an immunoglobulin molecule, e.g., IgG, is n-V_{L}-C_{L}-c and the structure of heavy chains of an immunoglobulin molecule, e.g., IgG, is n-V_{H}-C_{H1}-H-C_{H2}-C_{H3}-c (where H is the hinge region). The variable regions of the antibodies or antibody fragments include the complementarity determining regions (CDRs), which contain the residues in contact with antigen, and non-CDR segments, referred to as framework segments or framework regions (FRs or FwRs), which in general maintain the structure and determine the positioning of the CDR loops (although certain framework residues may also contact the antigen). Thus, the V_{L} and V_{H} domains have the structure n-FR1, CDR1, FR2, CDR2, FR3, CDR3, FR4-c.

Antibody fragments can be generated from an intact conventional IgG and include antigen-binding fragments, Fc domains, Fab fragments (F(ab)), F(ab′) fragments, single-chain Fv fragments (scFv) (VH-VL dimer), heavy chain domains only, light chain domains only, as well as individual (single) domains, e.g., VH domain, VL domain, CH₁ domain, CH₂ domain, CH₃ domain, CL domain, etc.

The terms “antibody single domain”, “single domain antibody”, or “sdAb” refer to antibody fragments that comprise or consist of either a VH or VL domain of an antibody, that is, a single monomeric variable antibody domain. Like an intact antibody, an antibody single domain can immunospecifically bind a specific antigen. Unlike whole antibodies, however, antibody single domains do not exhibit complement system triggered cytotoxicity, as they lack an Fc region. Two or more antibody single domains may combine to give dimers and higher order structures thereof.

As used herein, the terms “immunospecifically binds,” “immunospecifically recognizes,” “specifically binds,” “specifically recognizes” and analogous terms refer to molecules that specifically bind to an antigen (e.g., epitope or immune complex) and do not specifically bind to another molecule under physiological conditions. Molecules that specifically bind an antigen can be identified, for example, by immunoassays, BlAcore, or other techniques known to those of skill in the art. A molecule that specifically binds to an antigen may bind to other peptides or polypeptides but with lower affinity as determined by, e.g., immunoassays, BIAcore, or other assays known in the art. In some embodiments, molecules that specifically bind an antigen cross-react with other molecules (such as analogous protein from other species).

The term “in vivo half-life”, “serum half-life”, or “plasma half life” (also referred to as t_{1/2}) as used herein refers to a biological half-life of a molecule in the circulation of a given host and is represented by a time required for half the quantity administered in the animal to be cleared from the circulation and/or other tissues in the animal. The in vivo half-life is an important clinical parameter which determines the amount and frequency of administration for a therapeutic. When a clearance curve of a given molecule is constructed as a function of time, the curve is usually biphasic with a rapid “α-phase”, which represents an equilibration of the injected molecules between the intra- and extra-vascular space and which is, in part, determined by the size of molecules; and a longer “β-phase”, which represents the catabolism of the molecules in the intravascular space. In practical terms, the in vivo half-life usually corresponds closely to the half life of the molecules in the β-phase.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and derivatives of DNA or RNA molecules. Such derivatives can be generated using, for example, nucleotides, which include, but are not limited to, inosine or tritylated bases. Such derivatives can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably are double-stranded DNA.

An “isolated” or “purified” molecule is substantially free of cellular material or other contaminants from the cell or tissue source from which the molecule is derived, or substantially free of chemical precursors or other chemicals when chemically synthesized. The language “substantially free of cellular material” includes preparations of molecule in which the molecule is separated from cellular components of the cells from which it is isolated or recombinantly produced. Thus, a protein that is substantially free of cellular material includes preparations having less than about 30%, 20%, 10%, or 5% (by dry weight) of contaminating protein. When the molecule, typically a protein, is recombinantly produced, it is also typically substantially free of culture medium, i.e., culture medium represents less than about 30%, 20%, 10%, or 5% of the volume of the preparation. When the molecule is produced by chemical synthesis, it is typically substantially free of chemical precursors or other chemicals, i.e., it is separated from chemical precursors or other chemicals which are involved in the synthesis of the molecule. Accordingly such preparations have less than about 30%, 20%, 10%, 5% (by dry weight) of chemical precursors or compounds other than the molecule of interest. In one embodiment of the present invention, proteins, and in particular fusion proteins, are isolated or purified.

As used herein, the terms “subject,” “host” and “patient” are used interchangeably. A subject is typically a mammal such as a non-primate (e.g., cows, pigs, horses, cats, dogs, rats etc.) or a primate (e.g., monkey and human), most often a human.

### 2. Modified Albumin-Binding Domains

One aspect of the invention relates to albumin-binding domains, and fusions thereof, e.g., fusions with a biological therapeutic, such as a protein, e.g., an immuno-therapeutic, which have reduced immunogenicity and/or improved solubility. An albumin-binding domain or fusion thereof with reduced immogenicity is one in which at least one T_{H} epitope in the domain has been eliminated and/or reduced. Such a polypeptide, domain, or fusion is referred to herein as a “de-immunized” polypeptide, domain, or fusion. De-immunized albumin-binding domains and fusions, as well as albumin-binding fragments and derivatives thereof, in accordance with the invention, result in reduced immunogenicity in the intended host, e.g., in a human patient, as compared to the albumin-binding domain not de-immunized. Albumin-binding domains, with or without fusion to a therapeutic molecule, can be modified, where the modification reduces immunogenicity. In some embodiments, the therapeutic molecule of the fusion, e.g., a therapeutic polypeptide, may be de-immunized separately. In particular, the present invention encompasses variant albumin-binding domains, which have been modified to reduce immunogenicity of the variant.

Another aspect of the invention relates to albumin-binding domains, and fusions thereof, e.g., fusions with a biological therapeutic, such as a protein, e.g., an immuno-therapeutic, which have improved solubility, that is albumin-binding domains and fusions thereof, which has been mutated to enhance solubilty. Such a polypeptide, domain, or fusion is referred to herein as a “solubilized” polypeptide, domain, or fusion. Solubilized albumin-binding domains and fusions, as well as albumin-binding fragments and derivatives thereof, in accordance with the invention, result in good solubility in serum of the intended host, e.g., in a human patient, as compared to the albumin-binding domain not solubilized. Albumin-binding domains, with or without fusion to a therapeutic molecule, can be modified, where the modification improves or enhances solubility, thereby making the domain and/or fusions thereof, more soluble. In some embodiments, the therapeutic molecule of the fusion, e.g., a therapeutic polypeptide, may be modified separately or in addition to also improve its solubility. In particular, the present invention encompasses variant albumin-binding domains, which have been modified to enhance solubility of the variant. In some particularly preferred embodiments, the modified albumin-binding domains show both enhanced solubility and reduced immunogenicity.

In some particular embodiments, the albumin-binding domain is modified to reduce immunogenicity and/or enhance solubility by one or more amino acid substitutions. The amino acid substitutions for reducing immunogenicity in an albumin-binding domain, or albumin-binding fragment or derivative thereof, in accordance with the invention, may occur within stretches of amino acids that provide peptides with affinity for an HLA class II receptor—known as T_{H} epitopes. Substitution at a T_{H} epitope can eliminate or reduce binding to an HLA class II receptor, thus reducing the immunogenicity. For example, the substitution may occur within a strech of amino acids that provies a peptide with affinity for at least one HLA class II receptor selected from HLA class II receptors composed of DRA/DRB1, DQA/DQB and DPA/DPB.

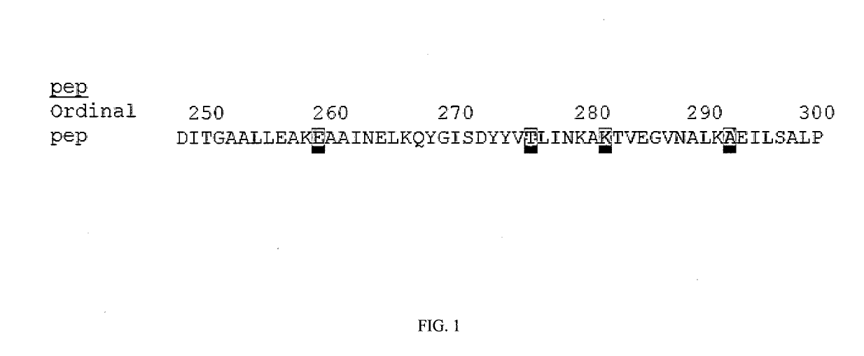

In particular embodiments, the albumin-binding domain to be de-immunized comprises or consists of an amino acid sequence corresponding to SEQ ID NO:1, which also may be referred to herein as “PEP.” De-immunized variants of PEP may be referred to herein as “PEP variants” See also Example 1 and FIG. 1.

In other particular embodiments, the PEP albumin-binding domain to be de-immunized comprises or consists of an albumin-binding fragment of SEQ ID NO:1. In some embodiments, the albumin-binding domain to be de-immunized comprises or consists of an albumin-binding fragment of PEP comprising or consisting of at least 10 contiguous amino acids of SEQ ID NO:1. In other embodiments, the albumin-binding domain to be de-immunized comprises an albumin binding-fragment of PEP comprising or consisting of at least 15 contiguous amino acids of SEQ ID NO:1. In other embodiments, the albumin-binding domain to be de-immunized comprises an albumin-binding fragment of PEP comprising or consisting of at least 20 contiguous amino acids of SEQ ID NO:1. In other embodiments, the albumin-binding domain to be de-immunized comprises an albumin-binding fragment of PEP comprising or consisting of at least 25 contiguous amino acids of SEQ ID NO:1. In other embodiments, the albumin-binding domain to be de-immunized comprises an albumin-binding fragment of PEP comprising or consisting of at least 30 contiguous amino acids of SEQ ID NO:1. In other embodiments, the albumin-binding domain to be de-immunized comprises an albumin-binding fragment of PEP comprising or consisting of at least 35 contiguous amino acids of SEQ ID NO:1. In other embodiments, the albumin-binding domain to be de-immunized comprises an albumin-binding fragment of PEP comprising or consisting of at least 40 contiguous amino acids of SEQ ID NO:1.

In still other embodiments, the albumin-binding domain to be de-immunized comprises or consists of at least two fragments of PEP that together bind albumin and that each independently include at least 10 contiguous amino acids of SEQ ID NO:1. In yet other embodiments, the albumin-binding domain to be de-immunized comprises at least two fragments of PEP that together bind albumin and that each independently include at least 15 contiguous amino acids of SEQ ID NO:1. In yet other embodiments, the albumin-binding domain to be de-immunized comprises at least two fragments of PEP that together bind albumin and that each independently include at least 20 contiguous amino acids of SEQ ID NO:1. In yet still further embodiments, the albumin-binding domain to be de-immunized comprises or consists of at least 10, at least 20, at least 30, at least 40, or at least 50 contiguous amino acids of SEQ ID NO:1.

In some embodiments, the albumin-binding domain to be de-immunized comprises or consists of an amino acid sequence corresponding to a derivative of the amino acid sequence of SEQ ID NO:1 . In some instances, the albumin-binding domain to be de-immunized has at least 45%, at least 50%, at least 55%, at least 60%, at least 65%, at least 70%, at least 75%, at least 80%, at least 85%, at least 90%, at least 95%, at least 98%, or at least 99% sequence identity to the amino acid sequence of SEQ ID NO:1.

In certain embodiments, the albumin-binding domain to be de-immunized comprises 1, 2, 3, 4, 5, 6, 7, 8, 9, 10, or more amino acid modifications (e.g., insertion, substitution, deletion, etc.) relative to the amino acid sequence of SEQ SEQ ID NO:1. Amino acid sequence derivatives of the albumin-binding domain can be created such that they are substitutional, insertional or deletion derivatives. Deletion derivatives lack one or more residues of the native polypeptide which are not essential for function (e.g., albumin-binding). Insertional mutants typically involve the addition of material at a non-terminal point in the peptide. Substitutional derivatives typically contain the exchange of one amino acid for another at one or more sites within the amino acid sequence, and may be designed to modulate one or more properties of the peptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Preferably, mutation of the amino acids of a protein creates an equivalent, or even an improved, second-generation molecule. For example, certain amino acids may be substituted for other amino acids in an amino acid sequence without detectable loss of function (e.g., albumin-binding). In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, interaction with a serum albumin molecule. Each amino acid has been assigned a hydropathic index on the basis of their hydrophobicity and charge characteristics; for example: isoleucine(+4.5); valine(+4.2); leucine(+3. 8); phenylalanine(+2.8); cysteine/cystine(+2.5); methionine(+1.9); alanine(+1.8); glycine(−0.4); threonine(−0.7); serine(−0.8); tryptophan 0.9); tyrosine(−1.3); proline(−1.6); histidine(−3.2); glutamate(−3.5); glutamine(−3.5); aspartate (-3.5); asparagine (−3.5); lysine (−3.9); and arginine (−4.5). It is also understood in the art that the substitution of like amino acids can be made effectively on the basis of hydrophilicity. Like hydrophobicity, values of hydrophilicity have been assigned to each amino acid: arginine (+3.0); lysine (+3.0); aspartate (+3.0±1); glutamate (+3.0+1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In general, equivalent molecules may be obtained by substitution of one amino acid for another where their hydrophilicity indices are within +2, preferably ±1, or most preferably ±0.5 of each other. The resulting derivatives may show improved solubility, improved stability and/or albumin binding, as well as reduced immunogenicity, or other advantegous feature described herein and/or known in the art. In particular, immmunogenicity can be reduced by amino acid substitutions identified by immunogenicity profiling analysis, as described above.

Accordingly, in some embodiments, PEP albumin-binding domains are provided that are de-immunized. In some embodiments, de-immunized PEP albumin-binding domains are provided that are in linked to a therapeutic molecule. The “de-immunized” domain will have been mutated to reduce T_{H} epitope content and will comprise one or more substitutions that reduce immunogenicity by reducing or eliminating epitopes that bind one or more HLA class II receptors. In some embodiments, de-immunized PEP albumin-binding domains further comprise mutations that facilitate expression and/or folding of the domain, and/or that facilitate expression and/or folding of a fusion comprising the de-immunized PEP albumin-binding domain and another agent, as described in more detail below.

In some embodiments, the de-immunized PEP albumin-binding domain comprises substitutions that eliminate at least 2 T_{H} epitopes, at least 3 T_{H} epitopes, at least 4 T_{H} epitopes, at least 5 T_{H} epitopes, at least 6 T_{H} epitopes, at least 8 T_{H} epitopes, at least 10 T_{H} epitopes, at least 12 T_{H} epitopes, or at least 15 T_{H} epitopes. In preferred embodiments, the substitutions do not affect, or at least do not substantially affect, binding of the domain or binding of a fusion protein containing the domain to serum albumin compared with binding before de-immunization.

Alternatively and/or in addition, the albumin-binding domain, or fusion thereof, may be modified to enhance solubility as compared to the wild type PEP albumin-binding domain and/or fusions thereof That is, in some embodiments, the amino acid substitutions result in increase solubility of the PEP albumin-binding domain and/or fusions thereof In some embodiments, the solubility of the agent may be increased by at least about 2 fold, at least about 5 fold, at least about 10 fold, at least about 15 fold, or more.

In particular embodiments, the PEP albumin-binding domain to be made more soluble comprises or consists of an amino acid sequence corresponding to SEQ ID NO:1 (PEP). Variants of PEP with increased solubility also may be referred to herein as “PEP variants” Accordingly, the invention encompasses variants of PEP albumin-binding domains, or albumin-binding fragments or derivative thereofs, that have been modified by any method known in the art and/or described herein to reduce immunogenicity and/or increase solubility.

In other particular embodiments, the PEP albumin-binding domain to be made more soluble comprises or consists of an albumin-binding fragment of SEQ ID NO:1. In some embodiments, the albumin-binding domain to be made more soluble comprises or consists of an albumin-binding fragment of PEP comprising or consisting of at least 10 contiguous amino acids of SEQ ID NO:1. In other embodiments, the albumin-binding domain to be made more soluble comprises an albumin binding-fragment of PEP comprising or consisting of at least 15 contiguous amino acids of SEQ ID NO:1. In other embodiments, the albumin-binding domain to be made more soluble comprises an albumin-binding fragment of PEP comprising or consisting of at least 20 contiguous amino acids of SEQ ID NO:1. In other embodiments, the albumin-binding domain to be made more soluble comprises an albumin-binding fragment of PEP comprising or consisting of at least 25 contiguous amino acids of SEQ ID NO:1. In other embodiments, the albumin-binding domain to be made more soluble comprises an albumin-binding fragment of PEP comprising or consisting of at least 30 contiguous amino acids of SEQ ID NO:1. In other embodiments, the albumin-binding domain to be made more soluble comprises an albumin-binding fragment of PEP comprising or consisting of at least 35 contiguous amino acids of SEQ ID NO:1. In other embodiments, the albumin-binding domain to be made more soluble comprises an albumin-binding fragment of PEP comprising or consisting of at least 40 contiguous amino acids of SEQ ID NO:1.

In still other embodiments, the albumin-binding domain to be modified to enhance solubility comprises or consists of at least two fragments of PEP that together bind albumin and that each independently include at least 10 contiguous amino acids of SEQ ID NO:1. In yet other embodiments, the albumin-binding domain to be made more soluble comprises at least two fragments of PEP that together bind albumin and that each independently include at least 15 contiguous amino acids of SEQ ID NO:1. In yet other embodiments, the albumin-binding domain to be made more soluble comprises at least two fragments of PEP that together bind albumin and that each independently include at least 20 contiguous amino acids of SEQ ID NO:1. In yet still further embodiments, the albumin-binding domain to be made more soluble comprises or consists of at least 10, at least 20, at least 30, at least 40, or at least 50 contiguous amino acids of SEQ ID NO:1.

In some embodiments, the albumin-binding domain to be made more soluble comprises or consists of an amino acid sequence corresponding to a derivative of the amino acid sequence of SEQ ID NO:1. In some instances, the albumin-binding domain to be made more soluble has at least 45%, at least 50%, at least 55%, at least 60%, at least 65%, at least 70%, at least 75%, at least 80%, at least 85%, at least 90%, at least 95%, at least 98%, or at least 99% sequence identity to the amino acid sequence of SEQ ID NO:1.

In certain embodiments, the albumin-binding domain to be made more soluble comprises 1, 2, 3, 4, 5, 6, 7, 8, 9, 10, or more amino acid modifications (e.g., insertion, substitution, deletion, etc.) relative to the amino acid sequence of SEQ ID NO:1. Amino acid sequence derivatives of the albumin-binding domain can be created such that they are substitutional, insertional or deletion derivatives, as described above with respect to de-immunized variants.

Alternatively and/or in addition, the albumin-binding domain, or fusion thereof, may be modified to reduce immunogenicity.

In particular embodiments, the PEP albumin-binding domain of the invention comprises an amino acid sequence corresponding to SEQ ID NO:1, or an albumin-binding fragment or derivative thereof, which is de-immunized and/or solubilized. In more particular examples, PEP is de-immunized by at least one amino acid substitution selected from the group consisting of of E12D, T29H-K35D, and A45D, where the numbering of the substitutions refer to amino acid positions in SEQ ID NO:1. In some particular embodiments, PEP is de-immunized by an amino acid substitution corresponding to E12D, where the numbering of the substitution refers to amino acid positions in SEQ ID NO:1. In some particular embodiments, PEP is de-immunized by a pair of amino acid substitutions corresponding to T29H-K35D, where the numbering of the substitutions refer to amino acid positions in SEQ ID NO:1. In some particular embodiments, PEP is de-immunized by an amino acid substitution corresponding to A45D, where the numbering of the substitution refers to amino acid positions in SEQ ID NO:1. In particular some embodiments, PEP is de-immunized by amino acid substitutions corresponding to E12D and T29H-K35D, where the numbering of the substitutions refers to amino acid positions in SEQ ID NO:1. In some particular embodiments, PEP is de-immunized by amino acid substitutions corresponding to E12D and A45D, where the numbering of the substitutions refers to amino acid positions in SEQ ID NO:1. In some particular embodiments, PEP is de-immunized by amino acid substitutions corresponding T29H-K35D and A45D, where the numbering of the substitutions refers to amino acid positions in SEQ ID NO:1. In some particular embodiments, PEP is de-immunized by amino acid substitutions corresponding E12D, T29H-K35D, and A45D, where the numbering of the substitutions refers to amino acid positions in SEQ ID NO:1. In some particularly preferred embodiments, PEP is de-immunized to comprise an amino acid sequence corresponding to SEQ ID NO:31. SEQ ID NO:31 refers to a particular de-immunized PEP variant, that also is referred to herein as “PEP DI.”

In preferred embodiments, the modifications that enhance solubility and/or decrease immunogenicity do not, or do not substantially, affect binding of the albumin-binding domain to albumin. In some instances, the affinity to albumin of the de-immunized and/or solubilized albumin-binding domains of the invention, albumin-binding fragment or derivatives thereof; or conjugates or fusions thereof with a therapeutic molecule, is at least 0.5×10⁹ M⁻¹. In other instances, the affinity is at least 1×10⁴ M⁻¹, at least 1×10⁵ M⁻¹, at least 1×10⁶ M⁻¹, at least 1×10⁻⁷ M⁻¹, at least 1×10⁸ M⁻¹, at least 1×10⁹ M⁻¹, at least 2×10⁹ M⁻¹, at least 3×10⁹ M⁻¹, at least 4×10⁹ M⁻¹, at least 5×10⁹ M⁻¹, at least 6×10⁹ M⁻¹, at least 7×10⁹ M⁻¹, at least 8×10⁹ M⁻¹, or at least 9×10⁹ M⁻¹. Albumin binding can be measured by any technique known in the art. In certain instances, albumin binding is measured by an in vitro assay such as those described in, for e.g. Epps, et al. (1999) J. Pharm. Pharmacol.51:41-48; Nguyen, et al. (2006) Prot. Engin. Design Select. 19:291-297; Weisiger, et al. (2001) J. Biol. Chem., 276:29953-29960.

The isolated albumin-binding domains, albumin-binding fragments or derivatives thereof; can be linked to one or more therapeutic molecules, before, during, or after modification to increase solubility and/or reduce immunogenicity. Therapeutic molecules include in particular antibodies or fragments thereof. In preferred embodiments, the linkage is a fusion resulting in a product that binds to serum albumin while maintaining original binding affinity to a respective antigen, and while not increasing immogenicity (or not increasing immunogenicity substantially) and/or while not decreasing solubility (or not decreasing solubility substantially), as described in more detail below.

### 3. Therapeutic Molecules

Another aspect of the instant invention relates to conjugates and/or fusions of therapeutic molecules with the modified PEP albumin-binding domains described herein. For example, de-immunized albumin-binding domains of PEP, albumin-binding fragments or derivatives thereof, can be used to make conjugates and/or fusions with therapeutic molecules to improve stability and/or serum half-life, without exacerbating immunogenicity, while maintaining (or substantially maintaining) bioavailability and/or bioactivity. Similarly, solubilized albumin-binding domains of PEP, albumin-binding fragments or derivatives thereof, can be used to make conjugates and/or fusions with therapeutic molecules to improve stability and/or serum half-life, without exacerbating insolubility, while maintaining (or substantially maintaining) bioavailability and/or bioactivity. Without being bound to theory, the strategy involves high-affinity non-covalent interaction with albumin to improve serum half-life, using a construct that has been de-immunized and/or made more soluble. Coupling an albumin-binding domain of the invention to a therapeutic molecule can allow in vivo association to serum albumins, which in turn can extend the half-life of the polypeptide, and/or improve storage and stability. Further, enhanced solubility promotes ease of manufacture, formulation, storage, and/or bioavailability. Accordingly, albumin-binding domains can be used to make conjugates and/or fusions with therapeutic molecules to improve storage, stability, solubility, and/or serum half-life, preferably while maintaining bioavailability and/or bioactivity, and without exacerbating immunogenicity.

A modified albumin-binding domain in accordance with the invention may be linked to a therapeutic molecule or therapeutic agent. Alternatively, an albumin-binding domain may be linked to a therapeutic molecule or therapeutic agent and then subjected to modification, e.g., to increase solubility and/or to decrease immunogenicity. “Therapeutic agent” is used interchangeably herein with “therapeutic molecule” and includes any agent that modifies a given biological response. Unless specifically indicated otherwise, “therapeutic agents” encompass agents that can be used prophylatically and/or that can be used diagnostically. Therapeutic agents also are not to be construed as limited to classical chemical therapeutic agents (chemotherapeutics). For example, the therapeutic agent may be a drug (e.g., a small organic molecule), drug moiety, radioactive material, macrocyclic chelator, siRNA molecule, or protein that modifies a biological response.

“Therapeutic proteins” are proteins, polypeptides, peptides, antibodies, or fragments or variants thereof, having one or more therapeutic and/or biological activities, in particular, a biological activity that is useful for treating, preventing, slowing, or ameliorating a disease. The term “therapeutic protein” is used interchangeably with “protein therapeutic agent.” A non-inclusive list of biological activities that may be possessed by a therapeutic protein includes: inhibition of H1V infection of cells, stimulation of intestinal epithelial cell proliferation, reducing intestinal epithelial cell permeability, stimulating insulin secretion, induction of bronchodilation and vasodilation, inhibition of aldosterone secretion, blood pressure regulation, promoting neuronal growth, enhancing an immune response, suppressing an immune response, decreasing platelet aggregation, receptor binding, enhancing or reducing inflammation, suppression of appetite, or any one or more of the biological activities described herein. Therapeutic proteins encompassed by the invention include but are not limited to, proteins, polypeptides, peptides, antibodies, and biologics. (The terms peptides, proteins, and polypeptides are used interchangeably herein.) In some embodiments, the therapeutic agent of the invention contains a fragment or variant of a therapeutic protein, such as a fragment or variant of an antibody. Additionally, the term “therapeutic protein” may refer to the endogenous or naturally occurring correlate of a therapeutic protein.

By a polypeptide displaying a “therapeutic activity” or a protein that is “therapeutically active” is meant a polypeptide that possesses one or more known biological and/or therapeutic activities associated with a therapeutic protein such as one or more of the therapeutic proteins described herein or otherwise known in the art. As a non-limiting example, a therapeutic protein is a protein that is useful to treat, prevent, slow, or ameliorate a disease, condition, or disorder. A therapeutic protein also may be one that binds specifically to a particular cell type (normal (e.g., lymphocytes) or abnormal e.g., (cancer cells)) and therefore may be used to target a compound (drug or cytotoxic agent) to that cell type specifically.

Protein therapeutic agents include, but are not limited to, tumor necrosis factor-alpha (TNF-α), anti-TNF-a antibodies and antibody fragments thereof, von Willebrand factor (vWF), anti-vWF antibodies and antibody fragments thereof, epidermal growth factor (“EGF”) or anti-EFG antibodies, protamine, protein A, a G protein, protein transduction domains (see e.g., Bogoyevitch et al., 2002, DNA Cell Biol 12:879-894, hereby incorporated by reference in its entirety); a toxin such as abrin, ricin, ricin A, pseudomonas exotoxin (e.g., PE-40), or diphtheria toxin; gelonin, and pokeweed antiviral protein; a protein such as tumor necrosis factor; interferons, including but not limited to α-interferon (IFN-α), β-interferon (IFN-β), nerve growth factor (NGF), platelet derived growth factor (PDGF), tissue plasminogen activator (TPA), an apoptotic agent (e.g., TNF-α, TNF-β, AIM I as disclosed in PCT Publication No. WO 97/33899), AIM II (see, e.g., PCT Publication No. WO 97/34911), Fas Ligand (Takahashi et al., J. Immunol., 6:1567-574, 1994), and VEGI (PCT Publication No. WO 99/23105), a thrombotic agent or an anti-angiogenic agent (e.g., angiostatin or endostatin); or a biological response modifier such as, for example, a lymphokine (e.g., interleukin-1 (“IL-1”), interleukin-2 (“IL-T”), interleukin-6 (“IL-6”), granulocyte macrophage colony stimulating factor (“GM-CSF”), and granulocyte colony stimulating factor (“G-CSF”)), macrophage colony stimulating factor (“M-CSF”), or a growth factor (e.g., growth hormone (“GH”)); a protease or a ribonuclease, antibody, monoclonal antibody, antibody fragment, single domain antibodies, dimers thereof, and other non-antibody proteins, e.g., a soluble receptor or receptor fragment, and an antigen of an infectious agent.

A protein therapeutic agent may also include a portion of a full length sequence. A non-exhaustive list of therapeutic protein portions includes, but is not limited to, IFNn, ANP, BINP, LANP, VDP, KUP, CNP, DNP, HCC-1, beta defensin-2, fractalkine, oxyntomodulin, killer toxin peptide, TIMP-4, PYY, adrenomedullin, ghrelin, CGRP, IGF-1, neuraminidase, hemagglutinin, butyrylcholinesterase, endothelin, and mechano-growth factor.

Non-protein therapeutic agents include, but are not limited to, antimetabolites (e.g., methotrexate, 6-mercaptopurine, 6-thioguanine, cytarabine, and 5-fluorouracil decarbazine), alkylating agents (e.g, mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), and the like.

Other specific examples of non-protein therapeutic agents include a cytotoxin (e.g., a cytostatic or cytocidal agent) or a radioactive element or a macrocyclic chelator. Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or derivatives thereof. Radioactive elements may include radionuclides (e.g., alpha-, beta-, gamma-emitters, etc.) known in the art for labeling (i.e., producing a detectable signal in vivo or in vitro) and/or producing a therapeutic effect (e.g., ¹²⁵I, ¹³¹I, ¹⁴C, etc.). Macrocyclic chelators may include those known in the art for conjugating radiometal ions. In certain embodiments, the macrocyclic chelator is 1,4,7,10-tetraazacyclododecane-N,N′,N″,N″-tetraacetic acid (DOTA), which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50, each incorporated by reference in their entireties.

In some embodiments, a non-protein therapeutic agent may itself be conjugated to another protein, e.g., a therapeutic protein, which may modify a given biological response. The conjugate then may be combined with a de-immunized albumin-binding domain within the scope of the invention. Techniques for conjugating therapeutic agents to proteins, such as antibodies, are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243 56, Alan R. Liss, Inc.; Hellstrom et al., “Antibodies For Drug Delivery”, in Controlled Drug Delivery (2nd Ed.), Robinson et al. (eds.), 1987, pp. 623 53, Marcel Dekker, Inc.; Thorpe, “Antibody Carriers Of Cytotoxic Agents In Cancer Therapy: A Review”, in Monoclonal Antibodies '84:Biological And Clinical Applications, Pinchera et al. (eds.), 1985, pp. 475 506; “Analysis, Results, And Future Prospective Of The Therapeutic Use Of Radiolabeled Antibody In Cancer Therapy”, in Monoclonal Antibodies For Cancer Detection And Therapy, Baldwin et al. (eds.),1985, pp. 303 16, Academic Press; and Thorpe et al., Immunol. Recombinant expression vector., 62:119 58, 1982.

In some embodiments, the therapeutic protein is itself modified to give a derivative, e.g., before, after, or during linkage to a modified albumin-binding domain in accordance with the invention. In particular, the present invention encompasses de-immunized PEP albumin-binding domain-bound therapeutic proteins that have been modified by any method known in the art and/or described herein that can increase or improve the serum half-life of the therapeutic protein, increase solubility, and/or reduce immunogenicity of the construct. For example, but not by way of limitation, derivatives include therapeutic proteins that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids. That is, another modification to extend the serum half-life of the molecules of the invention, to improve solubility, and/or to reduce immunogenicity, involves the use of non-natural amino acis, for example in the D form and/or the use of amino acid analogs, such as sulfur-containing forms of amino acids. In certain embodiments, reactive side chains of amino acid residues are capped, for example, the carboxy side chain in glutamic acid. Capping can be accomplished using any suitable capping groups, as known in the art. As a further example, the serum half-life of the de-immunized PEP albumin-binding domain-bound therapeutic protein may be increased or improved by including in the therapeutic protein an additional antigen binding domain, which domain immunospecifically binds to, e.g., fibronectin.

As a particular example, a therapeutic protein that is a cell surface or secretory protein may be modified by the attachment of one or more oligosaccharide groups. The modification, referred to as glycosylation, can dramatically affect the physical properties of proteins and can be important in protein stability, secretion, and localization. Glycosylation occurs at specific locations along the polypeptide backbone. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser or Asn-X-Thr sequence, where X can be any amino acid except proline. N-acetylneuramic acid (also known as sialic acid) is usually the terminal residue of both N-linked and O-linked oligosaccharides. Variables such as protein structure and cell type influence the number and nature of the carbohydrate units within the chains at different glycosylation sites. Glycosylation isomers are also common at the same site within a given cell type. Therapeutic proteins, such as cell surface and secretory proteins, for use in accordance with the instant invention, may be modified so that glycosylation at one or more sites is altered. This may be achieved by any techniques known in the art, including, e.g., by manipulation(s) of their corresponding nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine; or unglycosylated recombinant proteins may be produced by expressing the proteins in host cells that will not glycosylate them, e.g. in E. coli or glycosylation-deficient yeast.

The protein therapeutic agents for use in accordance with the invention may contain modifications to the C-and/or N-terminus which include, but are not limited to, amidation or acetylation, and that may also improve serum half-life. Acetylation refers to the introduction of a COCH₃ group and can occur either at the amino terminus or on a lysine side chain(s) of a protein or fragment thereof. Accordingly, in certain embodiments, the amino-terminal of the therapeutic protein, e.g., an immunospecific molecule, is modified by acetylation. In certain embodiments, a lysine side chain in the therapeutic protein, e.g., an immunospecific molecule, is modified by acetylation. In yet other embodiments, the therapeutic protein, e.g., immunospecific molecule, is acetylated both at the amino terminus and on one or more lysine side chains.

As another particular example, the serum half-life of proteins can also be increased by attaching polymer molecules such as high molecular weight polyethyleneglycol (PEG). PEG can be attached to polypeptides or fragments thereof with or without a multifunctional linker and either through site-specific conjugation of the PEG to the N- or C-terminus; or via epsilon-amino groups present on lysine residues. Linear or branched polymer derivatization that results in minimal loss of biological activity and/or minimal increase in immunogenicity can be used. The degree of conjugation can be closely monitored, e.g., by SDS-PAGE and mass spectrometry, to ensure proper conjugation of PEG molecules to the agents of the invention. Unreacted PEG can be separated from polypeptide-PEG conjugates by, e.g., size exclusion or ion-exchange chromatography.

Other methods known in the art to increase serum half-life include conjugation and/or fusion to antibody domains including, but not limited to, antibody constant regions including Fc and/or hinge regions (see for example, U.S. Pat. Nos. 5,565,335, and 6,277,375); and/or conjugation and/or fusion to interferon, thymosin targeting peptides, and/or permeability increasing proteins (see, e.g., U.S. Pat. Nos. 6,319,691 and 5,643,570). Modifications, such as glycosylation, amidation, acetylation, and PEGylation, may introduce additional immunogenetic sites, e.g., additional T_{H} epitopes, increasing immunogenicity of the construct such that additional de-immunization is desirable. Similarly, modifications, such as glycosylation, amidation, acetylation, and PEGylation, may reduce solubility of the construct such that additional modification to preserve or improve solubility is desirable.

The invention also encompasses the use of liposomes for prolonging or increasing the serum half-life of agents of the invention. In certain embodiments, the therapeutic protein, e.g., an immunospecific molecule comprising a VL or VH domain and an albumin-binding domain, may be conjugated to liposomes using previously described methods, see, e.g., Martin et al., 1982, J. Biol. Chem. 257: 286-288, which is incorporated herein by reference in its entirety. The invention also encompasses liposomes that are adapted for specific organ targeting, see, e.g., U.S. Pat. No. 4,544,545, or specific cell targeting, see, e.g., U.S. Patent Application Publication No. 2005/0074403.

The therapeutic agent that can be improved by combination with modified albumin-binding domains described herein can be any agent, but typically is an agent that is eliminated from the body in less than two weeks. In some embodiments, the agent is eliminated in less than one week. In specific embodiments, the agent is eliminated in less than 6 days, or in less than five days, or in less than four days, or in less than three days or in less than two days, or in less than one day. In more specific embodiments, the agent has a serum half life (t₁₁₂) of 24 hours or less, or of 23 hours or less, or of 22 hours or less, or of 21 hours or less, or of 20 hours or less, or of 19 hours or less, or of 18 hours or less, or of 17 hours or less, or of 16 hours or less, or of 15 hours or less, or of 14 hours or less, or of 13 hours or less, or of 12 hours or less, or of 11 hours or less, or of 10 hours or less, or of 9 hours or less, or of 8 hours or less, or of 7 hours or less, or of 6 hours or less, or of 5 hours or less, or of 4 hours or less, or of 3 hours or less, or of 2 hours or less.

In some embodiments, linkage of a modified (e.g., a de-immunized) albumin-binding domain of the invention alters the bioavailability of the therapeutic molecule, for example, increasing or decreasing bioavailability in terms of transport to mucosal surfaces, or other target tissues.

In certain embodiments, linkage to a modified (e.g., a de-immunized) PEP albumin-binding domain in accordance with the invention increases the half-life of the therapeutic agent in a host. In some embodiments, the half-life of the therapeutic agent in the host is increased by about 10%, by about 20%, by about 30%, by about 40%, by about 50%, by about 60%, by about 70%, by about 80%, by about 90%, by about 100%, by about 150%, by about 200%, by about 300%, by about 500%, by about 1000% or more. In other embodiments, linkage to a modified (e.g., a de-immunized) PEP albumin-binding domain in accordance with the invention increases the half-life of the therapeutic agent in a host by at least double, at least 3 times, at least 4 times, at least 5 times, at least 6 times, at least 7 times, at least 8 times, at least 9 times, at least 10 times, or more compared with the half-life of the agent alone (not linked to the modified albumin-binding domain).

In some embodiments, linkage to a modified (e.g., a de-immunized) PEP albumin-binding domain in accordance with the invention reduces elimination of the therapeutic agent from the host by at least 1 day, by at least 2 days, by at least 3 days, by at least 4 days, by at least 5 days, by at least one week, or more. In some embodiments, the therapeutic agent linked to a modified (e.g., a de-immunized) PEP albumin-binding domain in accordance with the invention has a serum half-life (t_{1/2}) of about 10 hours, about 20 hours, about 30 hours, about 35 hours, about 40 hours, about 45 hours, about 50 hours, about 55 hours, about 3 days, about 4 days, about 5 days, about 6 days, about 7 days, about 8 days, about 9 days, about 10 days, about 11 days, about 12 days, about 13 days, about 14 days, or more, such as for example, for about 3 weeks, about 4 weeks, or more. In some particular embodiments, the anti-TNF-alpha polypeptides linked to a modified albumin-binding domain in accordance with the invention have a half-life of from about 35 to about 42 hours, e.g. ,where the construct corresponds to SEQ ID NO:33. For comparison, in some particular embodiments, the anti-TNF-alpha polypeptide not linked to modified albumin-binding domain may have a half-life of only about 2 to about 5 hours, e.g., where the dimer corresponds to SEQ ID NO:32.

### 4. Antibodies

In specific embodiments, the therapeutic agent is a therapeutic protein comprising at least one antibody or fragment thereof, which may be conjugated and/or fused to a modified (e.g., a de-immunized) PEP albumin-binding domain in accordance with the invention. Such therapeutic agents may be referred to as “therapeutic antibodies” or “antibody therapeutic agents.” Fusion or conjugation to a de-immunized PEP albumin-binding domain in accordance with the invention provides a de-immunized PEP albumin-bindinng domain-antibody fusion or conjugate. Such antibody fusions and conjugates can be produced by any method known in the art, for example, fusions can be made by chemical synthesis or recombinant techniques. See Example 2.

Alternatively, at least one PEP albumin-binding domain, albumin-binding fragment or derivative thereof, is linked to a therapeutic protein comprising at least one antibody or fragment thereof, and the construct is modified, e.g., the construct is de-immunized, such that at least one T₁ epitope in the albumin-binding domain is reduced or eliminated. See Example 3.

In some preferred embodiments, the in vivo half-lives of the conjugated and/or fused antibodies or fragments thereof are extended, while the immunogenicity is reduced or at least not increased (or not substantially increased) compared to the un-fused or un-conjugated agent. In some preferred embodiments, the in vivo half-lives of the conjugated and/or fused antibodies or fragments thereof are extended, while the solubility is enhanced or at least not decreased (or not substantially decreased) compared to the un-fused or un-conjugated agent. See Examples 4 -6.

In particular embodiments, the antibody therapeutic agents are single domain antibodies, in particular, including dimers thereof, such as VL-VL dimers and VH-VH dimers. In particular embodiments, the antibody therapeutic agents are monoclonal antibodies, multispecific antibodies, humanized antibodies, synthetic antibodies, chimeric antibodies, polyclonal antibodies, single-chain Fvs (scFv), single chain antibodies, anti-idiotypic (anti-Id) antibodies (including, e.g., anti-Id and anti-anti-Id antibodies to antibodies of the invention), diabodies, minibodies, nanobodies, or antigen binding fragments of any of the above, including, but not limited to, Fab fragments, F(ab′) fragments, disulfide-linked bispecific Fvs (sdFv), and/or intrabodies. Antibody therapeutic agents may also include other portions of antibody molecules, e.g., Fc domain, CH₁ domain, CH₂ domain, CH₃ domain, CL domain, etc. The therapeutic agent can include immunoglobulin molecules that may be derived from any species (e.g., rabbit, mouse, rat), but are typically human immunoglobulin molecules. The immunoglobulin may be of any type (e.g., IgG, IgE, IgM, IgD, IgA and IgY), and/or class (e.g., IgG₁, IgG₂, IgG₃, IgG₄, IgA₁, and IgA₂) and/or subclass. Typically, the modified PEP albumin-binding domain-bound antibody, or antigen binding fragment thereof, binds the same epitope of an antigen, and in certain embodiments may be humanized.

Examples of monoclonal antibodies that may be suitable for use with the invention include, without limitation, infliximab, sold under the name RemicadeTM and used for treating rheumatoid arthritis and Crohn's disease; adaimumab, sold under the name Humira® and golimumab, sold under the name Simponi® both also used for treating rheumatoid arthritis; basiliximab, used for treating acute rejection of kidney transplants; bevacizumab (humanized), used as an anti-angiogenic cancer therapy; abciximab, used to prevent coagulation in coronary angioplasty; daclizumab (humanized), also used to treat acute rejection of kidney transplants; gemtuzumab (humanized), used to treat relapsed acute myeloid leukaemia; alemtuzumab (humanized), used to treat B cell leukemia; rituximab, used to treat non-Hodgkin's lymphoma; palivizumab (humanized) used to treat RSV infections in children; trastuzumab (humanized), sold under the name Herceptin® and used as an anti-cancer therapy for some types of breast cancer; nimotuzumab (humanized), currently under clinical trials, and the like.

In particularly preferred embodiments, the therapeutic agents comprise antibodies directed against TNF-α, as discussed in more detail below.

In certain embodiments, the therapeutic agents comprise soluble receptors, which may be conjugated and/or fused to a modified (e.g., a de-immunized) PEP albumin-binding domain in accordance with the invention. Such soluble receptor fusions and conjugates can be produced by any method known in the art, for example, fusions can be made by chemical synthesis or recombinant techniques. An example of a soluble receptor includes, e.g., etanercept, sold under the name Enbrel® and used to treat immune diseases such as rheumatoid arthritis, juvenile idiopathic arthritis, ankylosing spondylitis, psoriatic arthritis, and plaque psoriasis. In preferred embodiments, the in vivo half-lives of the conjugated and/or fused soluble receptors or fragments thereof are extended, while immunogenicity of the conjugate or fusion is reduced, not increased, or not substantially increased compared to the un-conjugated or un-fused agent. Alternatively, or in addition, in preferred embodiments, the in vivo half-lives of the conjugated and/or fused soluble receptors or fragments thereof are extended, while the solubility of the conjugate or fusion is increased, not decreased, or not substantially decreased, compared to the un-conjugated or un-fused agent.

In certain embodiments, agents comprising a modified (e.g., a de-immunized) PEP albumin-binding domain-bound antibody are bispecific or multispecific. Bi- or multi-specific molecules may be formed using methods well known in the art, e.g., fusion or chemical conjugation of one or more molecules described herein or known in the art to each other and/or to different epitope binding polypeptides, wherein the binding domains of the bi- or multi-specific molecule exhibit affinity for at least two different antigens. For example, the modified (e.g., de-immunized) PEP albumin-binding domain fusions with antibodies or antibody fragments may comprise a first and a second VL domain, or a first and second VH domain, wherein said first and second domain have different binding specificities (i.e., bind to different antigens). In other embodiments, the fusions can comprise one VL, or one VH domain, and/or one antigen binding polypeptide, wherein the VL domain, or VH domain, and/or antigen binding polypeptide exhibit different binding specificities (i.e. bind to different antigens). In some preferred embodiments, the fusions comprise two VL domains that both bind the same target, e.g., a VL-VL heteromer directed against TNF-alpha, as described in more detail below.

In certain embodiments, the agent comprising an antibody or antigen-binding fragment thereof bound to a modified (e.g., a de-immunized) PEP albumin-binding domain does not comprise a VH domain, e.g., a rabbit VH domain, and/or does not comprise a VH domain derived from any species other than rabbit. In other embodiments, the agent does not comprise a VL domain and/or does not comprise a VL domain derived from any species other than rabbit. In certain embodiments, the agent comprising a modified (e.g., a de-immunized) PEP albumin-binding domain bound to an antibody, or antigen binding fragment thereof, binds an epitope of an antigen which is immuno-neutral or non-immunogenic in certain species, e.g., mice and/or rats. In certain embodiments, the agent comprising an antibody or antigen-binding fragment thereof bound to a modified (e.g., a de-immunized) PEP albumin-binding domain does not comprise a VH domain, e.g., a human VH domain, and/or does not comprise a VH domain derived from any species other than human. In other embodiments, the agent does not comprise a VL domain and/or does not comprise a VL domain derived from any species other than human.

In certain embodiments, the agents comprising a modified (e.g., a de-immunized) PEP albumin-binding domain bound to an antibody, or antigen binding fragments thereof, do not comprise a CH₁ domain. In other embodiments, the agents do not comprise one or more of a CH₁ domain, CH₂ domain, CL domain, CH₃ domain, or H domain (hinge region), or do not comprise any of a CH₁ domain, CH₂ domain, CL domain, CH₃ domain, or H domain (hinge region). In still other embodiments, the agents comprise one of a CH₁ domain, H domain (hinge region), CH₂ domain, C_{L} domain, or CH₃ domain, and do not comprise any other constant domain or hinge region derived from an immunoglobulin (for example, in certain embodiments, the agent comprises a CH₁ domain, but does not comprise any of an H domain (hinge region), a CH₂ domain, or a CH₃ domain; or comprises a CH₂ domain, but does not comprise any of a CH₁ domain, H domain (hinge region), or a CH₃ domain, etc). In some embodiments, the agent comprising a modified (e.g., a de-immunized) PEP albumin-binding domain bound to an antibody, or antigen binding fragment thereof, comprises only a heavy chain, only a light chain, only a VH domain, only a VL domain, or any combination of the above fragments and/or domains.

In certain embodiments, the agents of the invention comprising modified (e.g., de-immunized) PEP bound to antibodies, or antigen binding fragments thereof, include one or more scaffold residues changes that improve stability and/or antigen binding affintity of the agents; and/or the reduce immunogenicity of the construct further. Standard techniques known to those skilled in the art can be used to introduce mutations in the nucleotide sequence encoding a therapeutic antibody, or fragment thereof, including, e.g., site-directed mutagenesis and PCR-mediated mutagenesis, which results in amino acid substitutions. In certain embodiments, the derivatives have conservative amino acid substitutions made at one or more predicted non-essential amino acid residues.

The present invention also encompasses modified (e.g., de-immunized) PEP albumin-binding domains linked to therapeutic agents and additional antigen-binding domains, e.g., heterologous polypeptides (i.e., an unrelated polypeptide; or portion thereof, typically at least 10, at least 20, at least 30, at least 40, at least 50, at least 60, at least 70, at least 80, at least 90, or at least 100 amino acids of the polypeptide) to generate fusion proteins. For example, the therapeutic proteins may be fused or conjugated to a antibody single domain or dimeric construct thereof, an Fab fragment, Fc fragment, Fv fragment, F(ab)₂ fragment, scFv, minibody, nanobody or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

By fusing or conjugating the modified (e.g., de-immunized) PEP albumin-binding domain-bound therapeutic molecule to other molecules that are specific for particular cell surface receptors, the therapeutic agent may be targeted to particular cell types, either in vitro or in vivo. Such fusions or conjugations can result in bispecific or multispecific polypeptides of the invention. In vitro uses include, e.g., in vitro immunoassays and purification methods using methods known in the art. See e.g., PCT Publication No. WO 93/21232; EP 439,095; Naramura et al., 1994, Immunol. Lett., 39:91-99; U.S. Pat. No. 5,474,981; Gillies et al., 1992, Proc Natl Acad Sci, 89:1428-1432; and Fell et al., 1991, J. Immunol., 146:2446-2452, each of which is incorporated herein by reference in its entirety.

In certain embodiments, the therapeutic proteins of the invention and/or fragments thereof are humanized. Humanization offers an alternate or additional strategy to reducing immunogenicity of agents of the invention. A humanized polypeptide is a polypeptide comprising at least one immunoglobulin variable domain (or a variant or fragment thereof) that is capable of immunospecifically binding to a predetermined antigen and that comprises a framework region (FR) having substantially the amino acid sequence of a human immunoglobulin and a CDR having substantially the amino acid sequence of a non-human immunoglobulin. In general, the humanized molecule, e.g., humanized antibody, will comprise substantially all of at least one, and typically two, variable domains, in which all or substantially all of the hypervariable regions correspond to those of a non-human immunoglobulin or variable domain; while all or substantially all of the framework regions are those of a human immunoglobulin sequence. For some uses of therapeutic proteins, and in particular antibodies, it may be preferable to use humanized molecules, e.g., in vivo use in humans and in vitro detection assays.

Generally, humanized antibodies are created by replacing hypervariable region residues of human immunoglobulins (or variable domains and/or fragments thereof) by hypervariable region residues from a non-human species (donor antibody; e.g., donor CDRs from a rabbit VH or VL domain) having the desired specificity, affinity, and/or capacity. One says that the donor molecule has been “humanized”, by the process of “humanization”, because the resultant humanized molecule is expected to bind to the same antigen as the donor antibody that provides the CDRs. For example, a humanized antibody may be constructed comprising one or more CDRs from a rabbit donor antibody and framework regions from a human immunoglobulin.

Humanized fusion or conjugates of a modified (e.g., a de-immunized) PEP albumin-binding domain with an antibody or antibody fragment may comprise a receptor antibody, e.g., selected from any class of human immunoglobulins, including IgM, IgG, IgD, IgA and IgE, and any isotype, including IgG₁, IgG₂, IgG₃ and IgG₄, in which one or more hypervariable region residues are replaced. In some instances, FR residues of the human immunoglobulin (receptor antibody), or fragment thereof, also are replaced by corresponding non-human residues. Often, framework residues in the framework regions are substituted with the corresponding residue from the CDR or variable domain donor to alter, preferably improve, antigen binding. These framework substitutions are identified by methods well known in the art, e.g., by modeling the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323, all of which are incorporated herein by reference in their entireties).

In some embodiments, the fusions or conjugates of modified (e.g., de-immunized) PEP albumin-binding domains with antibodies or antigen-binding fragments thereof comprise a humanized molecule wherein at least one CDR from a donor rabbit variable domain is grafted onto the recipient framework region. In other embodiments, at least two and more often, all three CDRs, of a donor rabbit VH and/or VL domains are grafted onto the recipient framework regions. In some embodiments, the therapeutic molecule does not comprise an entire immunoglobulin, or may comprise a single immunoglobulin variable domain (e.g., a V_{H} or V_{L} domain) or a dimer of two single domains, but may not comprise any other immunoglobulin domain or region (e.g., Fc, CH₁, CH₂, CH₃, CL, etc.).

Constant regions need not be present, but if they are, they are preferably substantially identical to human immunoglobulin constant regions, i.e., at least about 85-90%, typically about 95% or more identical. Hence, in accordance with embodiments wherein the therapeutic molecule comprises a humanized immunoglobulin, all parts of a said immunoglobulin, except the CDRs, are substantially identical to corresponding parts of natural human immunoglobulin sequences. For example, the humanized molecule may comprise a CL and/or a CH₁ domain and/or at least a portion of an immunoglobulin constant region (Fc) substantially identical to corresponding parts of natural human immunoglobulin sequences. Furthermore, humanized molecules may comprise residues which are not found in the recipient antibody or in the donor antibody. These modifications may be made to further refine functionality, e.g., immunospecificity. Further modifications may be made to reduce immunogenicity and/or enhance solubility in the intended host, e.g., a human, as detailed above.

In some embodiments, a humanized molecule of the invention is a derivative. Such a humanized molecule comprises amino acid residue substitutions, deletions, or additions in one or more of the non-human, e.g., rabbit, CDRs. The derivative of the humanized molecule of the invention may have substantially the same binding, better binding, or worse binding when compared to a non-derivative humanized molecule of the invention. In specific embodiments, one, two, three, four, or five amino acid residues of the CDRs have been substituted, deleted, or added (i.e., mutated). Such mutations, however, are usually not extensive. Usually, at least 75% of the humanized residues will correspond to those of the parental FR and CDR sequences, more often 90%, and most often greater than 95%. Other modifications encompassed by the term “humanized antibody”, as used herein, include methods of protein and/or antibody resurfacing such as those disclosed in U.S. Pat. Nos. 5,770,196; 5,776,866; 5,821,123; and 5,896,619, each to Studnicka et al. (each of which is incorporated herein by reference in its entirety).

In certain embodiments, a humanized molecule of the invention also comprises at least a portion of an immunoglobulin constant region (Fc), typically that of a human immunoglobulin. The constant domains of the humanized antibodies may be selected with respect to the proposed function of an antibody, in particular the effector function which may be required. In some embodiments, the constant domains of the humanized molecules of the invention are human IgA, IgE, IgG, or IgM domains. In a specific embodiment, human IgG constant domains, especially of the IgG₁ and IgG₃ isotypes, are used when the humanized molecules of the invention are intended for therapeutic uses and antibody effector functions are desired. For example, the constant domain may comprise a complement fixing constant domain where it is desired that the humanized molecule, e.g., antibody, exhibit cytotoxic activity, and the class is typically IgG₁. In alternative embodiments, IgG₂ and IgG₄ isotypes are used when the humanized molecule of the invention is intended for therapeutic purposes and antibody effector function is not required. For example, were cytotoxic activity is not desirable, the constant domain may be of the IgG₂ class. The humanized molecule of the invention also may comprise sequences from more than one class or isotype, and selecting particular constant domains to optimize desired effector functions is within the ordinary skill in the art.

Humanized molecules, in particular, antibodies, can be produced using a variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, Proc Natl Acad Sci USA 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596 (each of which is hereby incorporated by reference herein in its entirety). Humanized antibodies also may be obtained from transgenic mice and/or from libraries of human antibodies.

In some embodiments, the affinity of a therapeutic antibody for a target antigen and/or epitope is increased. For example, phage display technology can be used to increase the affinity of a therapeutic molecule for a target antigen and/or epitope. The technology, referred to as affinity maturation, employs mutagenesis or CDR walking and re-selection using target antigen and/or an epitope thereof to identify amino acid sequences of the invention that bind with higher affinity to the antigen when compared with the initial pool of selected sequences. Mutagenizing entire codons rather than single nucleotides results in a semi-randomized repertoire of amino acid mutations. Libraries can be constructed having a pool of variant clones each of which differs by a single amino acid alteration in a single CDR and which contain variants representing each possible amino acid substitution for each CDR residue. Mutants with increased binding affinity for the antigen can be screened, for example, by contacting the immobilized mutants with labeled antigen. Any screening method known in the art can be used to identify mutant antibodies with increased avidity to the antigen (e.g., ELISA) (See Wu et al., 1998, Proc Natl. Acad. Sci. USA 95:6037; Yelton et al., 1995, J. Immunology 155:1994, each of which is hereby incorporated by reference herein in its entirety).

The binding specificity of the polypeptides described herein may be evaluated by any method known in the art for determining binding-pair interactions, including, but not limited to ELISA, western blot, surface plasmon resonance (e.g., BlAcore), and radioimmunoassay. Any method known in the art for assessing binding polypeptide specificity may be used to identify polypeptides for use in accordance with the invention that exhibit a Kd of certain ranges, e.g., of greater than 0.001 nM but not greater than 5 nM, not greater than 10 nM, not greater than 15 nM, not greater than 20 nM, not greater than 25 nM, not greater than 30 nM, not greater than 35 nM, not greater than 40 nM, not greater than 45 nM, or not greater than 50 nM, e.g., as determined by BlAcore assay.

The present invention also provides for modified (e.g., de-immunized) PEP albumin-binding domain-antibody fusions and/or conjugates, or fragments thereof, that have a high binding affinity for a particular antigen of interest. In a specific embodiment, an agent of the present invention or fragment thereof has an association rate constant or kₘ, rate (antibody (Ab)+antigen (Ag) Ab-Ag) of at least 10⁵ M⁻¹ s⁻¹, at least 5×10⁵ M⁻¹ s⁻¹, at least 10^{6 M}⁻¹ s⁻¹ at least 5×10⁶ M⁻¹ s⁻¹, at least 10⁷ M⁻¹ s⁻¹ at least 5×10⁷ M⁻¹ s⁻¹ or at least 10⁸ M⁻¹ s⁻¹. In one embodiment, an agent of the present invention or fragment thereof has a kₒₙ of at least 2×10⁵ M⁻¹ s⁻¹, at least 5×10⁵ M⁻¹ s⁻¹, at least 10⁶ M⁻¹ s⁻¹, at least 5×10⁶ M⁻¹ s¹, at least 10⁷ M⁻¹ s⁻¹, at least 5×10⁷ M⁻¹ s¹, or at least 10⁸ M⁻¹ s⁻¹.

In particular embodiments, the modified (e.g., de-immunized) PEP albumin-binding domain of the invention, as well as albumin-binding fragments and derivatives thereof, is bound to an antibody or antibody fragment that specifically binds TNF-alpha, in particular human TNF-alpha. Such antibodies and fragments thereof are collectively referred to herein as called “anti-TNF-alpha polypeptides” and find use, e.g., in the treatment, prevention, delay,or management of condtions where the cytokine TNF-alpha is implicated as a causative agent. Without wishing to be bound by theory, the anti-TNF-alpha polypeptide may antagonize the binding of human TNF-alpha to its cognate receptor. The anti-TNF polypeptide preferably also cross-reacts with at least one non-primate mammalian TNF-alpha, facilitating pre-clinial testing in a mammal other than primates. In more preferred embodiments, the anti-TNF polypeptide cross-reacts with both at least one rodent and at least one non-rodent species. Rodent species include, e.g., rats, mice, squirrels, gerbils, porcupines, beavers, chipmunks, guinea pigs, and voles.

In some embodiments, the anti-TNF-alpha polypeptide that is fused to the modified (e.g., de-immunized) PEP albumin-binding domain of the invention is an antibody single domain, e.g., a light chain variable domain. As used herein “single domain antibody” is used interchangeably with “antibody single domain.” In other embodiments, the anti-TNF-alpha polypeptide is a dimer of two antibody single domains, e.g., a heterodimer of two light chain variable domains. In still other embodiments, the anti-TNF-alpha polypeptide comprises three or more antibody single domains, or antigen-binding fragments or derivatives of one, two, three or more antibody single domains that bind TNF-alpha. In some embodiments, the individual single domains are linked by a linker, e.g., a peptide linker or chemical linker. In some embodiments, the peptide linker also is de-immunized. In some embodiments, one or more of the antibody single domains also are de-immunized. Antibody single domains can be obtained from an animal immunized with the antigen TNF-alpha (e.g. a rabbit immunized with a human TNF-alpha molecule).

In some embodiments, single domains are selected that show high binding to human TNF-alpha, as well as cross-reactivity with rat and/or mouse TNF-alpha, or with TNF-alpha from one or more other non-human species, preferably including a relatively small mammal other than a primate, more preferably including one rodent and one non=rodent species. Relatively small mammals may include a rat, mouse, guinea pig, hamster, etc. This approach provides anti-TNF-alpha polypeptides suitable for use in the invention, where therapeutic effect can be achieved in human patients, while in vivo testing for such, in terms of, e.g., efficacy and toxicology assays, can be conducted in rat and/or mice models. That is, cross-reactivity facilitates cost reduction by allowing certain in vivo (pre-clinical) testing of putative therapeutic agents to be conducted in the animal whose TNF-alpha is a binding target for the putuative therapeutic, along with human TNF-alpha.

In some preferred embodiments, the anti-TNF-alpha polypeptide comprises a VL-VL heterodimer fused to a modified (e.g., a de-immunized) PEP albumin-binding domain, wherein one or both VL domains are de-immunized. In some even more preferred embodiments, the VL-VL heterodimer comprises one or both of SEQ ID NO:2 (VL18) or SEQ ID NO:3 (VL11), one or both of which also are de-immunized. In particular embodiments, the anti-TNF-alpha heterodimer is fused to a de-immunuzed PEP variant. In other particular embodiments, the anti-TNF-alpha polypeptide comprises either of VL18 or VL11 fused to a de-immunized PEP variant, where one or both of VL18 and VL11 also are de-immunized.

In particular embodiments, the anti-TNF-alpha polypeptide comprises a light chain variable domain, said variable domain comprising an amino acid sequence corresponding to SEQ ID NO:2 (VL18) or an antigen-binding fragment or derivative thereof, which is de-immunized, e.g., by at least one amino acid substitution selected from the group consisting of T7Q, V15P, (A51V-L54R/A51V-L54E), K63S, E79K, (C80S), T91A, and L111K. The numbering refers to amino acid positions of SEQ ID NO:2. Also, values between brackets refer to germline-filtered peptides, e.g. (C80S); double mutants are linked by a hyphen, e.g. A51V-L54R; and alternate proposed substitutions involving the same position are presented within brackets, separated by a slash, e.g. (A51V-L54R/A51V-L54E).

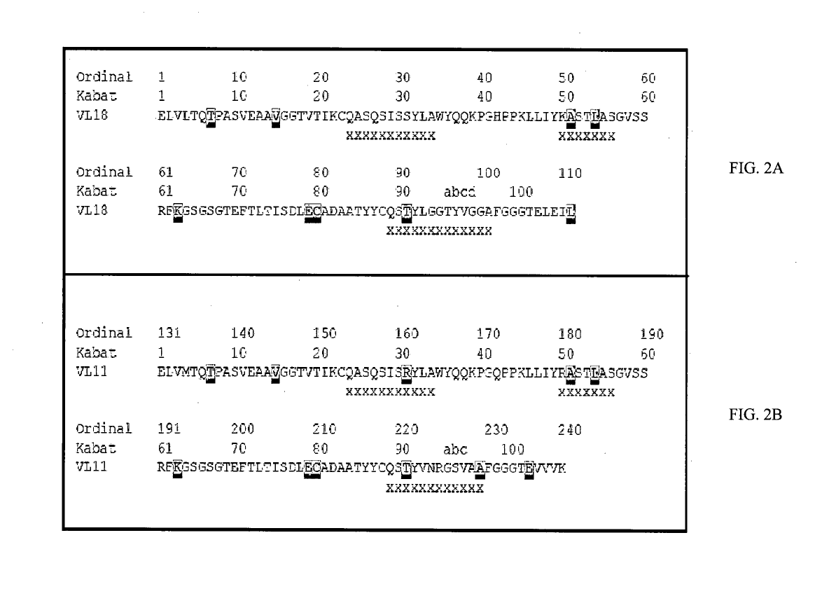

In particular embodiments, the anti-TNF-alpha polypeptide comprises a light chain variable domain, said variable domain comprising an amino acid sequence corresponding to SEQ ID NO:3 (VL11), or an antigen-binding fragment or derivative thereof, which is de-immunized e.g., by at least one amino acid substitution selected from the group consisting of T7Q, V15P, R31S, (A51V-54R /A51V-L54E), K63S, E79K, (C80S), T91A, A100S, and E106K (where the numbering refers to amino acid residues of SEQ ID NO:3). See Example 7 and FIGS. 2A-B.

In further specific embodiments, the anti-TNF-alpha polypeptide comprises a light chain variable domain, said variable domain comprising at least one sequence selected from the group consisting of SEQ ID NOs.:4-19 (other rabbit VLs), SEQ ID NOs:20-24 (five de-immunized VL 18 variants), SEQ ID NOs: 25-29 (five de-immunizecd VL11 variants), and a TNF-alpha-binding fragment or derivative thereof . In some specific embodiments, the anti-TNF-alpha polypeptide comprises a dimer of two light chain variable domains, one or both of said variable domains comprising at least one sequence selected from the group consisting of SEQ ID NO:2 (VL18), SEQ ID NO:3 (VL11), SEQ ID NOs.:4-19 (other rabbit VLs), SEQ ID NOs:20-24 (five de-immunized VL18 variants), SEQ ID NOs: 25-29 (five de-immunized VL11 variants), and a TNF-alpha-binding fragment or derivative thereof. The five de-immunized VL18 variants corresponding to SEQ ID NOs:20-24 may also be referred to herein as VL18 #1, VL18 #2, VL18 #3, VL18 #4, and VL18 #5, respectively. The five de-immunized VL11 variants corresponding to SEQ ID NOs:25-29 may also be referred to herein as VL11 #1, VL11 #2, VL11 #3, VL11 #4, and VL11 #5, respectively.

In particular embodiments, the anti-TNF-alpha polypeptide is coupled to an albumin-binding domain, said albumin-binding domain comprising an amino acid sequence corresponding to SEQ ID NO:1 (PEP), or an albumin-binding fragment or derivative thereof, which is de-immunized e.g., by at least one amino acid substitution selected from the group consisting of El2D, T29H-K35D, and A45D (where the numbering refers to amino acid residues of SEQ ID NO:1). In some particularly preferred embodiments, the anti-TNF-alpha polypeptide is coupled to an albumin-binding domain, said albumin-binding domain comprising an amino acid sequence corresponding to SEQ ID NO:31.

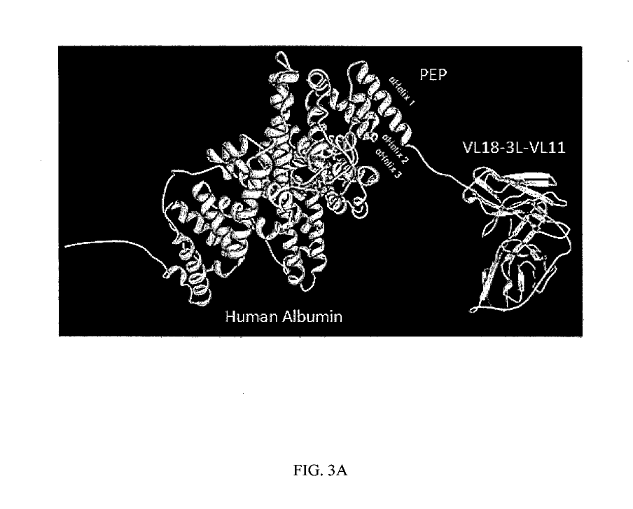

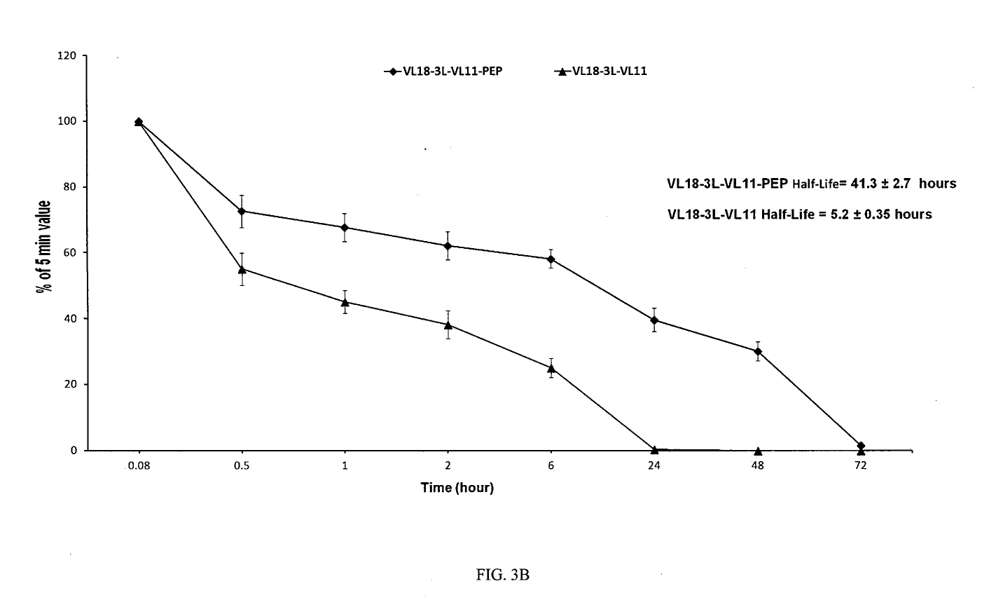

In further particular embodiments, the anti-TNF-alpha polypeptide comprises a dimer, wherein the dimer is bound to a modified (e.g., a de-immunized) PEP albumin-binding domain of the invention. In more particular embodiments, the dimer comprises an amino acid sequence corresponding to SEQ ID NO:32 (VL18-3L-VL11), an antigen-binding fragment or derivative thereof. In even more particular embodiments, agent of the invention comprises SEQ ID NO:33 (VL18-3L-VL11-PEP), or an antigen-binding fragment or derivative thereof, which further is de-immunized in PEP and/or other positions. For example, in specific embodiments, the agent of the invention comprises at least one amino acid sequence selected from the group consisting of SEQ ID NOs: 34-44 (eleven VL18-3L-VL11/PEP variants), or a TNF-alpha-binding fragment or derivative thereof. “3L” refers to a specific peptide linker, comprising the amino acid sequence corresponding to SEQ ID NO: 30. See Example 7 and FIGS. 3A-B.

In some embodiments, the agent of the invention comprises or consists of an amino acid sequence selected from the group consisting of SEQ ID NOs: 34-44. These sequences correspond to eleven VL18-3L-VL11/PEP variants, which have been de-immunized. SEQ ID NO:34 refers to a VL18-3L-VL11-PEP construct where PEP is de-immunized, as described herein, and also is referred to herein as “VL18-3L-VL11-PEP DI”. SEQ ID NOs:35-39 refer to de-immunized VL18-3L-VL11 constructs, which also may be referred to herein as VL18-3L-VL11 DI #1, VL18-3L-VL11 DI #2, VL18-3L-VL11 DI #3, VL18-3L-VL11 DI #4, and VL18-3L-VL11 DI #5, respectively. Any of the constructs corresponding to SEQ ID NOs:35-39 may be linked to a modified PEP albumin-binding domain, in accordance with the invention. For example, SEQ ID NOs:40-44 refer to de-immunized VL18-3L-VL11-PEP constructs, which also may be referred to herein as VL18-3L-VL11 DI #1-PEP DI, VL18-3L-VL11 DI #2-PEP DI, VL18-3L-VL11 DI #3-PEP DI, VL18-3L-VL11 DI #4-PEP DI, and VL18-3L-VL11 DI #5-PEP DI, respectively.

Modified fusions of the invention can be made by any technique known in the art or described herein, as detailed below.

### 5. Methods of Making Fusions or Conjugates

The present invention encompasses therapeutic molecules that are linked to a modified (e.g., de-immunized) PEP albumin-binding domain disclosed herein, or albumin-binding fragment or derivative thereof; or the therapeutic molecules may be otherwise engineered to comprise the modified albumin-binding domain, fragment, or derivative thereof. As noted above, linkage includes recombinant fusion and chemical conjugation (including both covalent and non-covalent conjugations). Linkage does not necessarily need to be direct, but may occur through a linker, such as peptide linker sequences, or through chemical conjugation. The linked agents can be referred to as “modified PEP albumin-binding domain fusions” or “modified PEP albumin-binding domain conjugates.” See Example 8-10.

Techniques for conjugating therapeutic moieties to polypeptides are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Helistrom et al., “Antibodies For Drug Delivery”, in Controlled Drug Delivery (2nd Ed.), Robinson et al. (eds.), 1987, pp. 623-53, Marcel Dekker, Inc.).

Protein linkers between modified (e.g., de-immunized) PEP albumin-binding domains and the therpeutic proteins of interest can be selected in order to maintain flexibility and proper folding, preferably such that the linked product shows binding to albumin, as well as to the original target of the therapeutic protein of interest (e.g., the antigen of an antibody or fragment thereof). Such binding assays are known to those of skill in the art.

The linker may be, e.g., a peptide linker or chemical linker. Examples of chemical linkers include, without limitation, a maleimide linker, a biocompatible polymer (preferably with a length of about 1 to about 100 atoms), aldehyde/Schiff base linkage, suphydryl linkage, and the like. See, e.g., US Patent Publication Application No. 2011/0196085 to Selinfreund.

Alternatively, a peptide linker may be used, e.g., where the polypeptide constructs are expressed as fusion proteins along with a connecting linker. Peptide linkers often comprise flexible amino acid residues, such as glycine and serine, so that adjacent domains are free to move relative to one another, as mentioned above. In some embodiments, the peptide linker used is 2 to 100 amino acids in length, 5 to 80, 10 to 50, 10 to 20, or 15 to 20 amino acids in length. Examples of peptide linkers include, without limitation, polyglycine, polyserine, polylysine, polyglutamate, polyisoleucine, or polyarginine residues, or combinations thereof. In a specific embodiment, the polyglycine or polyserine linkers include at least 5, 6, 7, 8, 9, 10, 12, 15, 20, 30, and 40 glycine and/or serine residues. In another specific embodiment, the linker involves repeats of glycine and serine residues, e.g., (Gly-Ser)ₙ residues, or (Gly-Ser)-residues with Glu or Lys residues dispersed throughout, e.g., to increase solubility. Other linkers comprising glycine and serine repeats include, e.g., (Gly)₄-Ser repeats; Gly-Gly-Ser-Gly repeats; Gly-Gly-Ser-Gly-Gly-Ser repeats; or (Gly)₄-Ser-(Gly)₃-Ser repeats; each at one, two, three, four, five, six, seven or more repeats. Standard linkers include (GGGGS); (GGGGS)₂; (GGGGS)₃; (GGGGS)₄; (GGGGS)₅; (GGGGS)₆. In some particular embodiments, the linker comprises repeats according to the formula [Ser-(Gly)₄]ₙ-(Ser)₂-Gly-, where n is 1, 2, 3, 4, 5, or 7. In other embodiments, n is 6, 8, 9, 10, or higher interger.

In another, specific embodiment, the linker includes proline and threonine residues, e.g., a ((PT)3T(PT)3T(PT)3S) linker. Additional peptide linkers are provided in Argos P. “An investigation of oligopeptides linking domains in protein tertiary structures and possible candidates for general gene fusion.” J Mol Biol 211(4): 943-58, 1990; Crasto, C J et al. “LINKER: a program to generate linker sequences for fusion proteins.” Protein Eng 13(5): 309-12, 2000; George, R A et al. “An analysis of protein domain linkers: their classification and role in protein folding.” Protein Eng 15(11): 871-9, 2002; and Arai R, et al. “Design of the linkers which effectively separate domains of a bifunctional fusion protein.” Protein Eng 14(8): 529-32, 2001, each of which is herein incorporated by reference in its entirety.

In some embodiments, a therapeutic protein is fused to modified (e.g., de-immunized) PEP albumin-binding domain according to the invention. Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers.

In some embodiments, the modified PEP albumin-binding domain fusion (or conjugate) includes at least a second therapeutic agent, different from a first therapeutic agent linked to the albumin-binding domain, or albumin-binding fragment or derivative thereof. The linked therapeutic agent may be referred to as a chimeric polypeptide. Methods for producing chimeric polypeptides, in particular antibodies, are known in the art. Once a nucleic acid sequence encoding an agent of the invention has been obtained, the vector for the production of the fusion with a modified PEP albumin-binding domain may be produced by recombinant DNA technology using techniques well known in the art.

Expression vectors containing the coding sequences of fused polypeptides in accordance with the invention, along with appropriate transcriptional and translational control signals, can be constructed using methods well known to those skilled in the art. An expression vector comprising the nucleotide sequence of an agent of the invention, e.g., a fusion protein with a modified (e.g., a de-immunized) PEP albumin-binding domain, can be transferred to a host cell by conventional techniques (e.g., electroporation, liposomal transfection, and calcium phosphate precipitation) and the transfected cells then can be cultured by conventional techniques to produce a polypeptide of the invention. Host-expression systems encompass vehicles by which the coding sequences of the polypeptides may be produced, and subsequently purified, and also encompass cells which may, when transformed or transfected with the appropriate nucleotide coding sequences, express the polypeptides of the invention in situ. The host cells used to express the recombinant fusions with modified (e.g., de-immunized) PEP albumin-binding domains of the invention may be, e.g., either bacterial cells such as Escherichia coli, or eukaryotic cells.

In a particular embodiment, E. coli Tuner™ (DE3) cells are used for large-scale expression of fusions of the invention. “Tuner™ strains” are lacZY deletion mutants of E. coli BL21 that facilitate controlled adjustment of the level of protein expression in cell culture. Expression levels are controlled by the lac permease (lacy mutation, which allows uniform entry of IPTG into cells in a population, producing a concentration-dependent, homogeneous induction in response to varying IPTG concentration. “DE3” indicates that the host is a lysogen of λDE3, carrying a chromosomal copy of the T7 RNA polymerase gene under control of the lacUV5 promoter.

Once a modified (e.g., a de-immunized) PEP albumin-binding domain fusion of the invention has been recombinantly expressed, it may be purified by any method known in the art for purification of an agent, for example, by chromatography (e.g., ion exchange, affinity, particularly by affinity for the specific antigen after Protein A, and sizing column chromatography), centrifugation, differential solubility, or by any other standard technique for the purification of proteins. Polypeptides of the invention can be fused to marker sequences, such as a peptide, to facilitate purification. In a particular embodiment, purification involves nickel affinity chromatography for endotoxin removal, following expression in E. coli . In another particular embodiment, purification involves Protein L and/or human albumin affinity chromatography, e.g., as described in more detail in the Examples below.

The modified (e.g., de-immunized) PEP albumin-binding domain fusions and/or conjugates constructed according to the invention, may be characterized for specific binding to a binding partner, e.g., in the case of antibodies, to an antigen and/or epitope, using any immunological or biochemical based method known in the art for characterizing binding-pair interactions. Specific binding may be determined for example using immunological or biochemical based methods including, but not limited to, an ELISA assay, surface plasmon resonance assays, immunoprecipitation assay, affinity chromatography, and equilibrium dialysis. Immunoassays which can be used to analyze immunospecific binding and cross-reactivity of the molecules of the invention include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety).

Modified (e.g., de-immunized) albumin-binding domain fusions and/or conjugates of the invention may also be assayed using any surface plasmon resonance based assays known in the art for characterizing the kinetic parameters of the interaction of an immunospecific protein with an antigen and/or epitope of interest. Any SPR instrument commercially available may be used including, but not limited to, BlAcore Instruments, available from Biacore AB (Uppsala, Sweden); IAsys instruments available from Affinity Sensors (Franklin, Mass.); IBIS system available from Windsor Scientific Limited (Berks, UK); SPR-CELLIA systems available from Nippon Laser and Electronics Lab (Hokkaido, Japan); and SPR Detector Spreeta available from Texas Instruments (Dallas, Tex.). For a review of SPR-based technology see Mullet et al., 2000, Methods 22: 77-91; Dong et al., 2002, Review in Mol. Biotech., 82: 303-23; Fivash et al., 1998, Current Opinion in Biotechnology 9: 97-101; Rich et al., 2000, Current Opinion in Biotechnology 11: 54-61; all of which are incorporated herein by reference in their entireties. Additionally, any of the SPR instruments and SPR-based methods for measuring protein-protein interactions described in U.S. Pat. Nos. 6,373,577; 6,289,286; 5,322,798; 5,341,215; 6,268,125 are contemplated in the methods of the invention, all of which are incorporated herein by reference in their entireties.

### 6. Polynucleotides Encoding the Polypeptides of the Invention

The invention provides polynucleotides comprising a nucleotide sequence encoding a polypeptide of the invention, such as a modified (e.g., de-immunized) PEP albumin-binding domain, albumin-binding fragment or derivative thereof, as well as fusions thereof to therpeutic proteins. In specific embodiments, the polynucleotide of the invention comprises or consists of a nucleic acid encoding a polypeptide disclosed herein, such as one or more of SEQ ID NOs:1-44. The invention also encompasses polynucleotides that hybridize under high stringency, intermediate or lower stringency hybridization conditions, to polynucleotides that encode a polypeptide of the invention.

The polynucleotides may be obtained, and the nucleotide sequence of the polynucleotides determined, by any method known in the art. For example, a polynucleotide encoding a polypeptide of the invention may be generated from nucleic acid from a suitable source (e.g., Streptococcal zooepidemicus). If a source containing a nucleic acid encoding a particular polypeptide is not available, but the amino acid sequence of the polypeptide of the invention is known, a nucleic acid encoding the polypeptide may be chemically synthesized and cloned into replicable cloning vectors using methods well known in the art.

Once the nucleotide sequence of the polynucleotide of the invention is determined, the nucleotide sequence may be manipulated using methods well known in the art for the cloning and manipulation of nucleotide sequences, e.g., recombinant DNA techniques, site directed mutagenesis, PCR, etc. As described above, such mutated sequences can provide polypeptides of the invention with enhanced pharmaceutical properties, e.g., improved serum half-life and/or reduced immunogenicity.

Alternatively a nucleic acid encoding the fusion product may be chemically synthesized. For example, using the desired amino acid sequence of the fusion polypeptide of the invention, the corresponding nucleotide sequence may be devised, chemically synthesized, and cloned into replicable cloning vectors using, e.g., well known methods in the art.

The invention further provides a vector comprising at least one polynucleotide encoding an agennt of the invention. In some embodiments, the vector is an expression vector. The invention further provides host cells comprising one or more vectors of the invention. The vectors, expression vectors, and host cells can include those discussed in detail above.

### 7. Methods of Use

The present invention encompasses therapies which involve administering an agent comprising at least one modified albumin-binding domain of PEP, or albumin-binding fragment or derivative thereof, linked to a therapeutic molecule to a host for preventing, treating, or ameliorating symptoms associated with a disease, disorder, or infection. Fusions and/or conjugates with a modified (e.g., de-immunized) PEP albumin-binding domains of the present invention that function as prophylactic and/or therapeutic agents against a disease, disorder, or infection can be administered to a host, particularly to a human, to treat, prevent or ameliorate one or more symptoms associated with the disease, disorder, or infection. As well as protein-based fusions and molecule-based conjugates, prophylactic and therapeutic agents of the invention include, but are not limited to, nucleic acids encoding fusion proteins and conjugated molecules. The agents may be provided as pharmaceutically acceptable compositions as known in the art and/or as described herein. The fusions and/or conjugates with a modified (e.g., de-immunized) PEP albumin-binding domain of the invention may be administered alone or in combination with other prophylactic and/or therapeutic agents. In preferred embodiments, the host or subject is a human, e.g., a patient in need of at least one modified albumin-binding domain of PEP, or albumin-binding fragment or derivative thereof, linked to a therapeutic molecule. In a particularly preferred embodiment, the present invention is directed to the treatment of a human subject, e.g., by administering an agent comprising at least one modified albumin-binding domain of PEP, or albumin-binding fragment or derivative thereof, linked to the therapeutic molecule (or nucleic acid encompassing same), in accordance with the instant disclosure, to a human subject in need thereof.

As used herein, the terms “therapeutic agent” refers to any agent which can be used in treating or amelioring symptoms associated with a disease, disorder, condition or infection. As used herein, a “therapeutically effective amount” refers to the amount of agent that provides at least one therapeutic benefit in the treatment or management of a disease, when administered to a subject suffering therefrom. Further, a therapeutically effective amount with respect to an agent of the invention means that amount of agent alone, or in combination with other therapies, that provides at least one therapeutic benefit in the treatment or management of a disease or condition.

As used herein, the term “prophylactic agent” refers to any agent which can be used in the prevention or delay of a disorder, or prevention or delay of a disease, disorder, condition, or infection, or slowing down the progression of the disease, disorder, condition, or infection, or prevention of recurrence or spread thereof. A “prophylactically effective amount” refers to the amount of the prophylactic agent that provides at least one prophylactic benefit in the prevention or delay of disease, when administered to a subject predisposed thereto. A prophylactically effective amount also may refer to the amount of agent sufficient to prevent or delay the occurrence of a disease, disorder, condition, or infection in a subject, or slow the progression of the disease, disorder, condition, or infection, or the amount sufficient to delay or minimize the onset of the disease, disorder, condition, or infection; or the amount sufficient to prevent or delay the recurrence or spread thereof. A prophylactically effective amount also may refer to the amount of agent sufficient to prevent or delay the exacerbation of symptoms of a disease, disorder, condition, or infection. Further, a prophylactically effective amount with respect to a prophylactic agent of the invention means that amount of prophylactic agent alone, or in combination with other agents, that provides at least one prophylactic benefit.

A prophylactic agent of the invention can be administered to a subject “pre-disposed” to a disease, disorder, condition, or infection. A subject that is “pre-disposed” to a particular disease, disorder, condition, or infection is one that shows symptoms associated with the development of the disease, disorder, condition, or infection, or that has a genetic makeup, environmental exposure, or other risk factor for such a disease, disorder, condition, or infection, but where the symptoms are not yet at the level to be diagnosed as the disease, disorder, condition, or infection. For example, a patient with a family history of rheumatoid arthritis may qualify as one predisposed thereto.

In some embodiments, methods for enhancing the efficacy of a therapeutic molecule in a subject are provided. The method may comprise providing an agent comprising a therapeutic molecule linked to at least one modified albumin-binding domain of PEP, or an albumin-binding fragment or derivative thereof; and administering the agent to the subject. Efficacy may be enhanced, e.g., in terms of the amount and/or frequency and/or duration of dosage of therapeutic agent needed to achieve a given beneficial result, while not exacerbating immunogenicity. Efficacy may be said to be doubled where the total dose required to achieve a given result is halved. Without wishing to be bound by theory, the linkage to an albumin-binding domain as taught herein may increase serum half-life of a therapeutic agent, while not affecting or not substantially affecting, its therapeutic properties, such as its bioavailability and/or bioactivity, with the result that less needs to be administered. Further, de-immunization reduces immunogenicity of the construct and/or the same or other modification may improve solubility. For example, in certain embodiments, linkage to a de-immunized PEP albumin-binding domain in accordance with the invention increases the efficcacy of the therapeutic agent in a host. In some embodiments, the efficacy of the therapeutic agent in the host is increased by about 10%, by about 20%, by about 30%, by about 40%, by about 50%, by about 60%, by about 70%, by about 80%, by about 90%, by about 100%, by about 150%, by about 200%, by about 300%, by about 500%, by about 1000% or more. Such altered pharmacokinetics also may allow for a change in a therapeutic regimen.

Accordingly, in some embodiments, methods for altering a therapeutic regimen for a therapeutic molecule are provided, wherein the alteration is a reduction in the amount of the molecule that is required to treat a condition. Reduction in the amount of molecule can refer to a reduction in the dosage of the therapeutic molecule, and/or a reduction in the frequency of dosage, and/or a reduction in duration of the regimen, such that treatment may end sooner. Use of reduced amounts can reduce adverse side effects, reduce medical costs, and/or improve patient compliance. As noted above, the reduction may be due to linkage to an albumin-binding domain in accordance with the invention, where the linkage extends the serum half-life of the therapeutic molecule in vivo, preferably without decreasing or without substantially decreaseing its therapeutic properties. For example, in certain embodiments, linkage to de-immunized PEP albumin-binding domain in accordance with the invention reduces the total dosage of therapeutic agent. The dosage may be reduced by about 10%, by about 20%, by about 30%, by about 40%, by about 50%, by about 60%, by about 70%, by about 80%, by about 90%, by about 100%, by about 150%, by about 200%, by about 300%, by about 500%, by about 1000%, or more. Enhanced solubility can further reduce the required doses. Moreover, due to the use of de-immunized domains, reduction in dosage is achieved without increasing, or without substantially increasing, the immunological response to the agent in the host upon administration thereof.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regime of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combintions thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, prophylactic and/or therapeutic agents are tested in a mouse model system. Such model systems are widely used and well known to the skilled artisan.

In some particular embodiments, animal model systems for a TNF-alpha related condition are used that are based on rats, mice, or other small mammal other than a primate. For example, in a specific embodiment, putative prophylactic and/or therapeutic anti-TNF-alpha polypeptide bound to a de-immunized albumin-binding domain of the invention is tested in a mouse or rat model system, such as, e.g., the established rat-adjuvant-induced arthritis (AIA) model or the collagen-induced arthritis (CIA) model. The AIA model is a much more aggressive model of arthritis compared to the CIA model. Testing in such systems, rather than primate-based model systems, afford the advantage of reduced costs in in vivo and/or pre-clinical testing. Without wishing to be bound by theory, testing in animals other than primates is feasible where anti-TNF-alpha antibody molecules are selected based on cross-reactivity with rat and/or mouse TNF-alpha, or with TNF-alpha of another non-primate, small mammal. Such antibodies provide agents suitable for use in the invention, where therapeutic effect can be achieved in human patients, while in vivo testing, e.g., testing for low toxicity, can be conducted in rat and/or mice models.

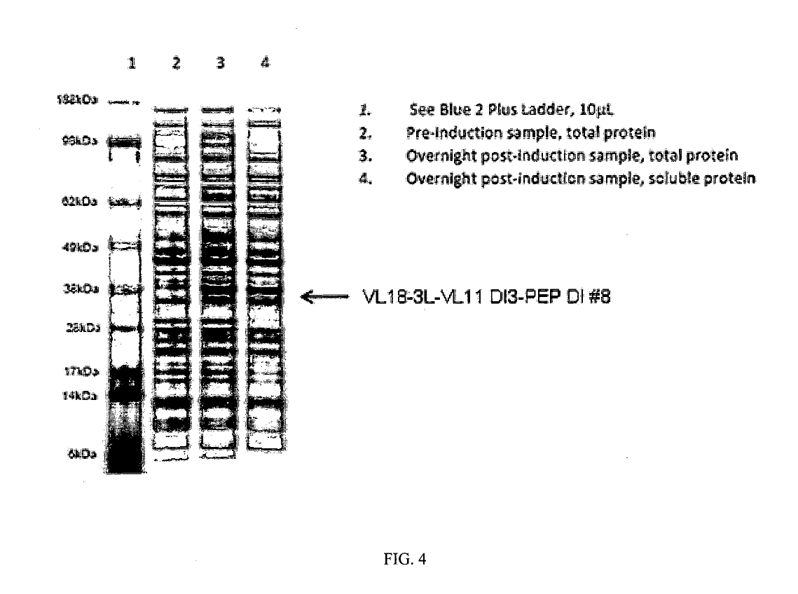

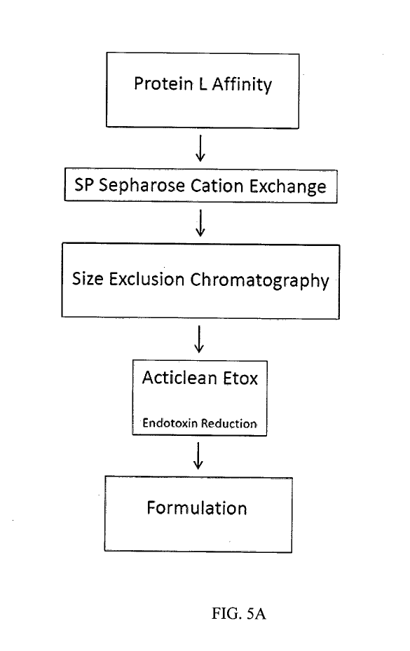

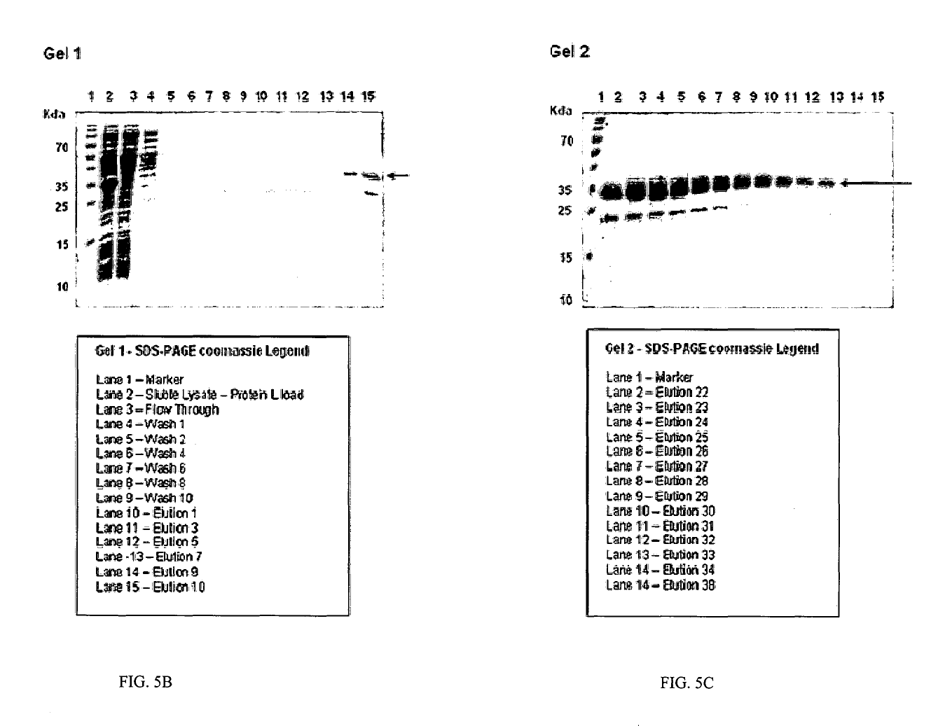

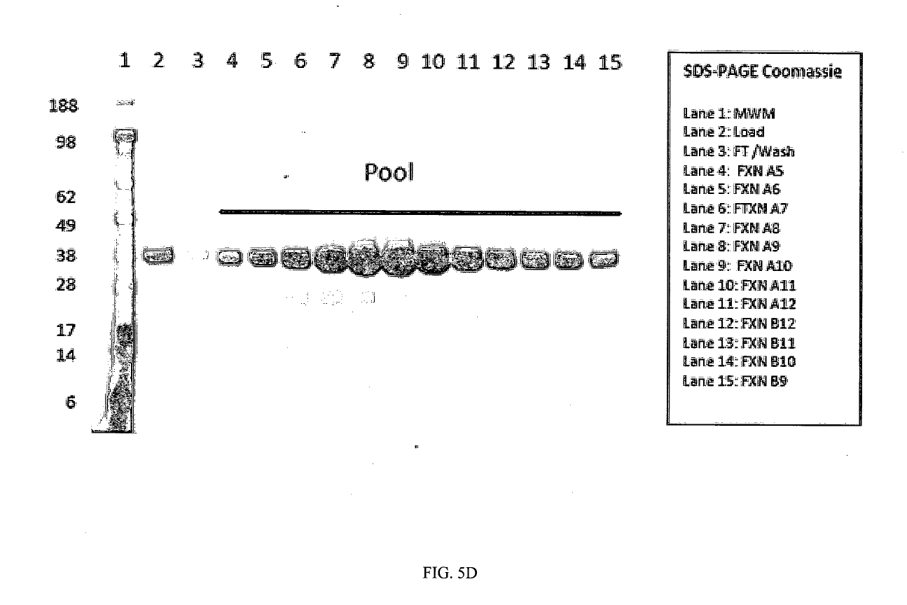

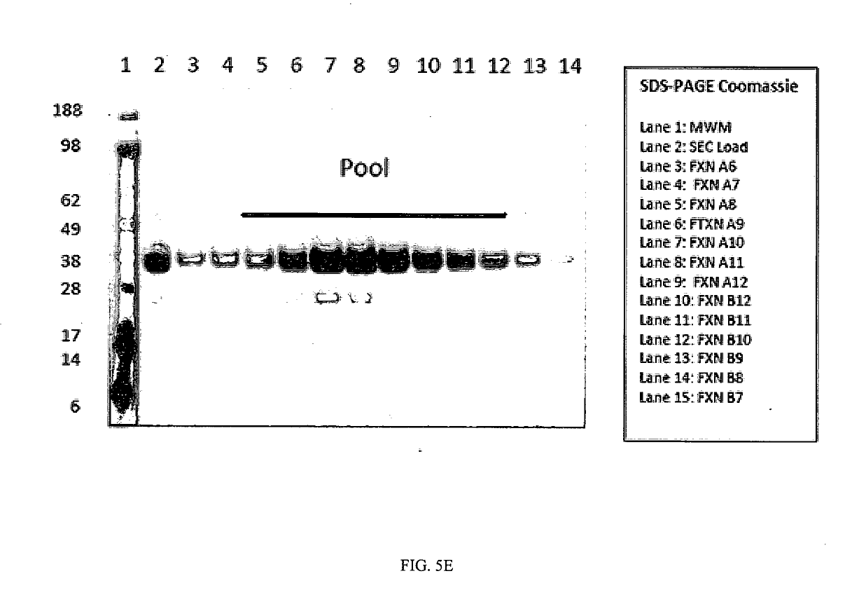

Another animal model involves the transgenic mouse (Tg197) model. The Tg197 model of arthritis is a humanized TNF transgenic mouse model with human TNF-alpha deregulated expression resulting in the spontaneous development of arthritis pathology closely resembling that of the human rheumatoid arthritis (Keffer et al. 1991 “Transgenic mice expressing human tumor necrosis factor: a predictive genetic model of arthritis” The EMBO Journal 10(13): 4025-4031, the contents of which are hereby incorporated by reference in entirety). The Tg 197 mouse develops chronic polyarthritis with 100% incidence at 4-7 weeks of age and provides a fast in vivo model for assessing human therapeutics for the treatment of rheumatoid arthritis. This model was successfully used in establishing the therapeutic efficacy of Remicade™ and is currently widely used for efficacy studies testing bio-similars or novel anti-human TNF-alpha therapeutics. See also Examples 11a-b and FIGS. 4-5.

Once the prophylactic and/or therapeutic agents of the invention have been tested in an animal model they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a fusion of an anti-TNF-alpha polypeptide with a modified (e.g., de-immunized) albumin-binding domain of the invention for efficacy and toxicity against rheumatoid arthritis in human patients. See Examples 12-19 below.

In some embodiments, an anti-TNF-alpha polypeptide is administered in a total dose of about 0.1 ng to about 1 g to treat a TNF-alpha-related condition, in a human patient, such as rheumatoid arthritis. In more particular embodiments, anti-TNF-alpha polypeptide is administered in a total dose of about 0.1 μg to about 1 mg; about 1 μg to about 500 μg; about 10 μg to about 400 μg; or about 50 μg to about 200 μg.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized.

Therapeutic or prophylactic agents of the present invention that function as antagonists of a disease, disorder, condition, or infection can be administered to a host to treat, prevent or ameliorate one or more symptoms associated with the disease, disorder, condition, or infection. For example, agents comprising a modified (e.g., de-immunized) PEP albumin-binding domain according to the invention linked to an antibody or antigen binding fragment thereof can be used as antagonists against viral infection. That is, modified PEP albumin-binding domain fusions with antibodies or antigen binding fragments thereof, where the antibody or fragment disrupts or prevent the interaction between a viral antigen and its host cell receptor, may be administered to a host to treat, prevent or ameliorate one or more symptoms associated with the viral infection.

In a specific embodiment, a modified PEP albumin-binding fusion with an antibody or antigen binding fragment thereof prevents a viral or bacterial antigen from binding to its host cell receptor by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to antigen binding to its host cell receptor in the absence of the antibody fusion. In another embodiment, a combination of antibody fusions prevent a viral or bacterial antigen from binding to its host cell receptor by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to antigen binding to its host cell receptor in the absence of the antibody fusions.

Fusions of a modified PEP albumin-binding domains with antibodies or antigen binding fragments thereof, which do not prevent a viral or bacterial antigen from binding its host cell receptor, but inhibit or downregulate viral or bacterial replication can also be administered to an animal to treat, prevent or ameliorate one or more symptoms associated with the viral or bacterial infection. The ability of an antibody to inhibit or downregulate viral or bacterial replication may be determined by techniques described herein or otherwise known in the art. For example, the inhibition or downregulation of viral replication can be determined by detecting the viral titer in the animal.

In a specific embodiment, a fusion of a modified PEP albumin-binding domain with an antibody or antigen binding fragment thereof downregulates viral or bacterial replication by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to viral or bacterial replication in absence of the antibody fusion. In another embodiment, a combination of antibody fusions inhibit or downregulate viral or bacterial replication by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to viral or bacterial replication in absence of the combination of antibody fusions.

Fusions with a modified PEP albumin-binding domains of the invention can also be used to prevent, inhibit or reduce the growth or metastasis of cancerous cells. In a specific embodiment, the fusion inhibits or reduces the growth or metastasis of cancerous cells by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to the growth or metastasis in absence of the fusion. In another embodiment, a combination of fusion agents in accordance with the invention inhibits or reduces the growth or metastasis of cancer by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to the growth or metastasis in absence of the combination of fusions. Examples of cancers include, but are not limited to, leukemia (e.g, acute leukemia such as acute lymphocytic leukemia and acute myelocytic leukemia), neoplasms, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease, metastases, or any disease or disorder characterized by uncontrolled cell growth.

Fusions of a modified PEP albumin-binding domains of the invention can also be used to reduce the inflammation experienced by animals, particularly mammals, with inflammatory symptoms and/or disorders. For example, fusions with a modified PEP albumin-binding domains that include a region that functions as an agonist of the immune response can be administered to treat, prevent or ameliorate one or more symptoms associated with an immune condition. In a specific embodiment, a fusion in accordance with the invention reduces the inflammation in an animal by at least 99%, at least 95%, at least 90%, at least 85%, at least 80%, at least 75%, at least 70%, at least 60%, at least 50%, at least 45% at least 40%, at least 35%, at least 30%, at least 25%, at least 20%, or at least 10% relative to the inflammation in an animal not administered the fusion. In another embodiment, a combination of fusions with a modified PEP albumin-binding domains reduce the inflammation in an animal by at least 99%, at least 95%, at least 90%, at least 85%, at least 80%, at least 75%, at least 70%, at least 60%, at least 50%, at least 45%, at least 40%, at least 35%, at least 30%, at least 25%, at least 20%, or at least 10% relative to the inflammation in an animal in not administered the combination of fusions. Examples of inflammatory disorders include, but are not limited to, rheumatoid arthritis, spondyloarthropathies, inflammatory bowel disease, Crohn's disease, multiple sclerosis, and asthma.

In specific embodiments, a fusion of a a modified PEP albumin-binding domain, albumin-binding fragment or derivative thereof, with an anti-TNF-alpha polypeptide can be administered to treat, delay, prevent, slow, or ameliorate one or more symptoms associated with rheumatoid arthritis. Rheumatoid arthritis is characterized by inflammatory responses in the synovial joints (synovitis), leading to destruction of cartilage and ankylosis of the joints. Synovitis involves inflammation of the synovial membranes lining joints and tendon sheaths. The affected joints become swollen, tender, and stiff. Other characteristic symptoms of rheumatoid arthritis include rheumatoid nodules, which can be a few millimeters to a few centimeters in diameter, often subcutaneous, and generally found over bony prominences; as well as vasculitis, which leads to a purplish discoloration of the skin. Lungs, kidneys, heart and blood vessels may also be affected. For example, fibrosis of the lungs and pleural effusions are associated with rheumatoid arthritis; and renal amyloidosis can occur due to chronic inflammation. Also, rheumatoid arthritis patients are more prone to artheroscleorsis, myocardial infarction, and stroke.

In preferred embodiments, an agent in accordance with the invention reduces inflammation in a synovial joint in an animal by at least 99%, at least 95%, at least 90%, at least 85%, at least 80%, at least 75%, at least 70%, at least 60%, at least 50%, at least 45% at least 40%, at least 35%, at least 30%, at least 25%, at least 20%, or at least 10% relative to the inflammation in a synovial joint in an animal not administered the agent. In particularly preferred embodiments, reduction in inflammation leads to reduced swelling, tenderness, and/or stiffness in the affected joint. In some preferred embodiments, an agent in accordance with the invention reduces rheumatoid nodules in an animal, e.g., in terms of their frequency over body prominences and/or their size. In some preferred embodiments, an agent in accordance with the invention reduces vasculitis, e.g, reducing the extent and/or degree of discoloration of vasculitis over the skin.

In another specific embodiment, fusions of a modified PEP albumin-binding domains with immunoglobulins are used in passive immunotherapy (for either therapy or prophylaxis). In a specific embodiment, fusions of a modified PEP albumin-binding domains with antibodies are administered to an animal that is of a species origin or species reactivity that is the same species as that of the fused antibody. Thus, in one embodiment, fusions of a modified PEP albumin-binding domains with human or humanized antibodies are administered to a human patient for therapy or prophylaxis.

As another specific example, fusions of a modified PEP albumin-binding domains with natriuretic peptides may be used for the treatment of cardiovascular disorders. For example, in one embodiment, fusions of a modified PEP albumin-binding domains with natriuretic peptides are used for the treatment of one or more of congestive heart failure, post-myocardial infarction, hypertension, salt-sensitive hypertension, angina pectoris, peripherial artery disease, hypotension, cardiac volume overload, cardiac decompensation, cardiac failure, non-hemodynamic CHF, left ventricular dysfunction, dyspnea, myocardial reperfusion injury, left ventricular remodeling, and/or elevated aldosterone levels, which can lead to vasoconstriction, impaired cardiac ouput, and/or hypertension.

In still another specific embodiment, fusions of a modified PEP albumin-binding domains linked to siRNA can be useful for the treatment or prevention of diseases that are caused by over-expression or misexpression of genes and diseases brought about by expression of genes that contain mutations. The mechanisms of siRNA activity and it mode of use are well known in the art, see, e.g., Provost et al., 2002, EMBO J., 21: 5864-5874; Tabara et al., 2002, Cell 109:861-71; Ketting et al., 2002, Cell 110:563; and Hutvagner & Zamore, 2002, Science 297:2056, each of which is hereby incorporated by reference herein in its entirety.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising an antibody or antigen binding fragment thereof, may increase or decrease over the course of treatment.

In some embodiments, ongoing treatment is indicated, e.g., on a long-term basis, such as in the ongoing treatment and/or management of chronic inflammatory disorders, such as rheumatoid arthritis. For example, in particular embodiments, an agent of the invention is administered over a period of time, e.g., for at least 6 months, at least one year, at least two years, at least five years, at least ten years, at least fifteen years, at least twenty years, or for the rest of the lifetime of a subject in need thereof.

Therapeutic or prophylactic agents of this invention may also be advantageously utilized in combination with one or more other drugs used to treat the particular disease, disorder, or infection such as, for example, anti-cancer agents, anti-inflammatory agents, or anti-viral agents.

Therapeutic or prophylactic agents of the invention can be administered in combination with one or more other prophylactic and/or therapeutic agents useful in the treatment, prevention or management of a disease, disorder, or infection. For example, the fusions and/or conjugates with a modified PEP albumin-binding domain of the invention may be administered alone or in combination with other prophylactic and/or therapeutic agents. Combined effects may be additive or synergistic. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, no more than 48 hours apart, no more than 72 hours apart, no more than 96 hours apart, no more than 5 days apart, no more than 6 days apart, no more than a week apart, no more than 2 weeks apart, no more than three weeks apart, no more than a month apart, no more than two months apart, or no more than three months apart. In certain embodiments, two or more agents are administered within the same patient visit.

The invention provides methods of treatment, prophylaxis, and amelioration of one or more symptoms associated with a disease, disorder, or infection by administering to a subject an effective amount of an agent comprising a a modified PEP albumin binding domain, or an albumin-binding fragment or derivative thereof, linked to a therapeutic molecule; or by administering a pharmaceutical composition comprising at least one of the agents of the invention.

Various delivery systems are known and can be used to administer agents of the invention, e.g., encapsulation in liposomes, microparticles, microcapsules, recombinant cells capable of expressing the fusions of a modified PEP albumin-binding domains (See, e.g., Wu and Wu, 1987, J. Biol. Chem. 262:4429-4432), construction of a nucleic acid as part of a retroviral or other vector, etc. Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In a specific embodiment, the agents of the invention are administered intramuscularly, intravenously, or subcutaneously, and may be administered together with other biologically active agents. In a more specific embodiment, fusions comprising an anti-TNF-alpha polypeptide and a modified PEP albumin-binding domain of the invention are formulated for subcutaneous administration as a sterile product. Administration can be systemic or local.

In a specific embodiment, it may be desirable to locally administer an agent of the invention or pharmaceutical composition comprising same to an area in need of treatment; this may be achieved by, for example, and not by way of limitation, local infusion, by injection, or by means of an implant, said implant being of a porous, non-porous, or gelatinous material, including membranes, such as sialastic membranes, or fibers. Typically, when administering an agent comprising an antibody, or antigen binding fragment thereof, care must be taken to use materials to which the antibody or antigen binding fragment does not absorb.

In another embodiment, the agent can be delivered in a vesicle, in particular a liposome (see Langer, Science, 249:1527 1533, 1990; Treat et al., in Liposomes in the Therapy of Infectious Disease and Cancer, Lopez-Berestein and Fidler (eds.), Liss, New York, pp. 353 365 (1989); Lopez-Berestein, ibid., pp. 3 17 327; see generally ibid.).

In another specific embodiment, agents of the invention comprising immunospecific molecules may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the immunospecific molecules of the invention are enclosed within a device such that release of the molecules is controlled by permeation through a difussion barrier. Common reservoir devices include, for example, membranes, capsules, microcapsules, liposomes, and hollow fibers. Monolithic (matrix) device are a second type of diffusion controlled system, wherein the immunospecific molecules are dispersed or dissolved in an rate-controlling matrix (e.g., a polymer matrix). Agents of the invention comprising immunospecific portions can be homogeneously dispersed throughout a rate-controlling matrix and the rate ofelease is controlled by diffusion through the matrix. Polymers suitable for use in the monolithic matrix device include naturally occurring polymers, synthetic polymers and synthetically modified natural polymers, as well as polymer derivatives.

The present invention also encompasses agents comprising a a modified PEP albumin-binding domain linked to a diagnostic agent. For diagnostic applications, such as detectable substance. Such molecules can be used diagnostically to, for example, monitor the development or progression of a disease, disorder or infection as part of a clinical testing procedure to, e.g., determine the efficacy of a given treatment regimen.

Examples of detectable substances include various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride, or phycoerythrin; luminescent material such as, but not limited to, luminol; bioluminescent materials such as, but not limited to, luciferase, luciferin, and aequorin; radioactive materials such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰1 Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and/or nonradioactive paramagnetic metal ions. Additionally, any of the above tracer radio metals may exhibit therapeutic effect as well when conjugated to a a modified PEP albumin-binding domain in accordance with the methods of the invention.

The detectable substance may be coupled or conjugated either directly or indirectly to a a modified PEP albumin-binding domain or a therapeutic agent itself linked thereto, such as an antibody or antigen binding fragment thereof. See, for example, U.S. Pat. No. 4,741,900 (hereby incorporated by reference in its entirety) describing conjugation of metal ions to antibodies for use as diagnostics.

### 8. Pharmaceutical Compositions and Kits

The invention further provides a pharmaceutical composition comprising a pharmaceutically acceptable carrier and an agent, said agent comprising at least one modified (e.g., de-immunized) albumin-binding domain of PEP, or an albumin-binding fragment or derivative thereof, linked to a therapeutic molecule. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetabl,e or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients and stabilizers include, but are not limited to buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In preferred embodiment, the agent of the invention is substantially purified (i.e., substantially free from substances that limit its effect or produce undesired side-effects). In a specific embodiment, the host or subject is an animal, preferably a mammal such as non-primate (e.g., cows, pigs, horses, cats, dogs, rats etc.) and a primate (e.g., monkey such as, a cynomolgous monkey and a human). In a preferred embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a disease, in one or more containers

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. In an alternative embodiment, an agent of the invention is supplied in liquid form in a hermetically sealed container indicating the quantity and concentration of agent or active agent. Typically, the liquid form of the agent is supplied in a hermetically sealed container at at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

In some embodiments, prophylactic or therapeutic agents of the invention are administered subcutaneously. Subcutaneous administration allows for fast delivery of the agent, e.g., within a few minutes after subcutaneous administration. In particular embodiments, for example, a fusion of a therapeutic molecule with a modified PEP albumin-binding domain of the invention is provided with a pharmaceutically acceptable carrier, wherein the carrier is suitable for subcutaneous administration. Generally, the carrier will be sterile and have an osmolality compatible with administration into the blood.

Kits and devices facilitating self-administration also are preferred in some embodiments. For example, a “pen” as used with respect to delivery of insulin or Humira, may be used to effect subcutaneous delivery of an agent of the invention. (See, e.g., McCoy E K, et al. 2010 “A Review of Insulin Pen Devices” Postgrad Med 122(3): 81-88; and Pearson, T L. 2010 “Practical Aspects of Insulin Pen Devices,” Symposium, J. of Diabetes Sci and Tech, 4(3): 522-531, which each are hereby incorporated-by-reference in their entireties). The pen device for delivery of a fusion of a therapeutic molecule with a de-immunuzed albumin-binding domain of the invention may be disposable or reusable, and may come prefilled or designed for use with cartridges of the agent. Prefilled disposable pens on the market include those sold under ther brand names Humalog®, KwikPen®, Humulin®, Lantus®, Apidra®, Levemir®, Novolog®, FlexPen®, and the like. Refillable pens on the market include those sold under the brand names Autopen®, HumaPen®, LUXURA™, NovoPen®, and OptiClik®, and the like. Pen devices provide an alternative to vial-and-syringe approaches for self-administration of therapeutic agents by the subcutaneous route, and can offer a number of advantages including greater ease of use, greater portability and convenience, improved dosing accuracy, less pain, greater social acceptance, greater discreteness, and greater patient compliance.

In some embodiments, the pen has a dial-back feature, allowing for reversing the amount to be administered by “dialing back”. In some embodiments, the pen has a memory and digital display of the last dose or a last number doses. Pens normally are kept at room temperature, and inusulated storage packs may be used where pens are stored in places subjected to extremes of temperature. The pen device may be sold with or separately from pen needles. Needles may come in a variety of gauges and/or lengths. Gauges can be about 25 to about 35 gauge, such as 29 gauge, 30 gauge, 31 gauge, or 32 gauge, and about 2 mm to about 30 mm in length, e.g., about 3 mm, about 5 mm, about 10 mm, about 12.7 mm, about 15 mm, about 20 mm, or about 25 mm in length.

Generally, the ingredients of compositions of the invention are supplied either separately or mixed together in unit dosage form, for example, as a dry lyophilized powder or water free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EXAMPLES

### Example 1

### Sequencing of De-immunized Albumin-Binding Domains

A de-immunized albumin-binding domain (PEP) was sequenced. The de-immunized domain contained four subsitutions as follows: E12D, T29H-K35D, and A45D, where the numbering refers to amino acid positions in SEQ ID NO:1. In the listing, the double mutants are linked by a hyphen, e.g. T29H-K35D. The proposed positions for substitutions are highlighted in gray. Sequences of the PEP domain containing the substitutions are presented in FIG. 1.

### Example 2

### Construction of De-Immunized Albumin-Binding Domain Fusions

DNA fragments comprising de-immunized albumin-binding domains, and albumin-binding fragments or derivatives thereof, are generated using PCR and Spel and NcoI restriction sites were addded at the fragment 5′ and 3′ ends, respectively. The resulting PCR fragments are gel-purified, digested with the Spel and NcoI restriction enzymes and cloned into the phagemid vector pComb3X. The amino acid sequence of the expressed peptides comprise SEQ ID NO:1 (PEP), further comprising at least one amino acid substitution selected from the group consisting of E12D, T29H-K35D, and A45D, where the numbering refers to amino acid positions in SEQ ID NO:1 (PEP), as well a albumin-binding fragments or derivatives thereof. These expressed peptides are referred to as PEP variants.

The PEP variants were fused to several small domain antibody fragments (sdAbs) to give antibody fusions in order to test whether the sdAb fusions can retain binding affinity to their respective antigens, while binding to human, rat, and/or mouse serum albumin also is accomplished. Fusion proteins were prepared with PEP variants at either the N-terminal region or the C-terminal region of the sdAb.

To express and purify the fusion proteins, pET28a plasmid containing each gene was used to transform E. coli Tuner (DE3) cells.

A fresh colony of each clone was grown at 37° C. overnight in SB medium supplemented with kanamycin. A 10 ml sample of cells was used to inoculate 1 L of SB medium supplemented with kanamycin. Cells were grown at 37° C. until A_{600 nm} reached 0.9. Antibody expression was induced by the addition of 0.1 mM isopropyl-1-thio-β-D-galactoside (IPTG) and growth was continued for 18 hours, at 16° C. After induction, bacteria were harvested by centrifugation (4,000×g, 4° C., 15 min) and resuspended in 50 ml equilibration buffer (20 mM Hepes, 1 M NaCl, 30 mM imidazole, 10% glycerol, pH 7.4) supplemented with protease inhibitors (Roche). Cells were lysed by sonication. Centrifugation (14000×g, 4° C., 30 min) was used to remove cellular debris, and the supernatant was filtered through a 0.2-μm syringe filter.

All chromatographic steps were performed at 4° C. First, antibody fragments extracts were purified by nickel chelate affinity chromatography using the C-terminal His-tag. Bound proteins were eluted with a linear imidazole gradient from 0 to 300 mM imidazole in 20 mM Hepes, 0.5 M NaCl; 10% glycerol (pH 7.4). The appropriate fractions were pooled, concentrated and then purified by size exclusion chromatography using Superdex column and Hepes buffer (20 mM Hepes, 200 mM NaCl, 5% glycerol, pH 7.4). Purified antibody fragments were analyzed by SDS-PAGE followed by Coomassie-Blue staining and western-blot with HRP-conjugated anti-HA monoclonal antibody. Protein concentration was determined by measuring the optical density at 280 nm.

### Example 3

### Alternative Construction of De-Immunized Albumin-Binding Domain Fusions

DNA fragments comprising albumin-binding domains, and albumin-binding fragments or derivatives thereof, were generated using PCR and SpeI and NcoI restriction sites addded at the fragment 5′ and 3′ ends, respectively. The resulting PCR fragments were gel-purified, digested with the SpeI and NcoI restriction enzymes and cloned into the phagemid vector pComb3×. The amino acid sequence of the expressed peptides comprise SEQ ID NO:1 (PEP), or albumin-binding fragments or derivatives thereof.

The peptides corresponding to SEQ ID NO:1, and albumin-binding fragments or derivatives thereof, were fused to several small domain antibody fragments, or dimers thereof (e.g., VL-VL sdAbs) to give PEP fusions, and albumin binding-fragments or derivatives thereof. An in silico assessment against human T cell epitopes was carried out as a de-immunizing strategy of the PEP fusions, and albumin binding-fragments or derivatives thereof, to reduce T_{H} epitope content.

A list of amino acid substitutions was proposed:

Proposed Amino Acid Substitutions

In the listing below, the double mutants are linked by a hyphen, e.g. T29H-K35D. Four amino acid substitutions were proposed—in PEP: E12D, T29H-K35D, and A45D.

Amino acid substitutions also are proposed in one or more of the sdAb domains.

The de-immunized PEP fusions, and albumin-binding fragments or derivatives thereof, were expressed as described above. The amino acid sequence of the expressed peptides comprises SEQ ID NO:1 (PEP), further comprising at least one amino acid substitution selected from the group consisting of E12D, T29H-K35D, and A45D, where the numbers refer to amino acid positions in SEQ ID NO:1 (PEP), as well as albumin-binding fragments or derivatives thereof. These expressed peptides also are referred to as PEP variants.

The PEP variants were fused to several small domain antibody fragments (sdAbs) to give antibody fusions in order to test whether the sdAb fusions can retain binding affinity to their respective antigens, while binding to human, rat, and/or mouse serum albumin also is accomplished. Fusion proteins were prepared with PEP vairants at either the N-terminal region or the C-terminal region of the sdAb. The fusion proteins were expressed and purified as described above.

### Example 4

### De-Immunized Albumin-Binding Domain Fusions Bind Serum Albumin

Binding of the fusions of Examples 2 and 3 to albumin was tested by ELISAs. Binding ELISAs were performed as described briefly: albumin from human, rat, or mouse sera, at 10 mg/ml, was immobilized overnight in 96 well-plates at 4° C. After 2 h blocking with PBS/3% soy milk, recombinant antibody fragments (sdAbs) and fusions thereof to de-immunized albumin-binding domains were incubated for 1 h at room temperature. After washing the wells with PBS, anti-HA-HRP mAb (Roche) was used for detection. Optical density at 405 nm was measured and assays performed in triplicate.

The binding studies compare binding of PEP variant-sdAb fusions with unfused sdAbs to each of human, rat, and mouse sera albumin. The fusions show increased serum albumin binding to each of human, mouse, and rat albumin at one or more concentrations. That is, albumin-binding by a fusion according to the invention shows several fold improvements, e.g., by five or six fold or more, compared to the unfused sdAb of interest.

Competition ELISAs further demonstrate albumin binding of the fusions according to the invention. Competition ELISAs are performed as described briefly: PEP variant-sdAb fusions at increasing concentrations are pre-incubated with 10 μg of each of the different albumins for 1h at room temperature and subsequently added to the microtiter plates coated with the corresponding albumins. Detection is performed with mouse HRP conjugated anti-HA-tag antibody and absorbance is read at 405 nm. In each instance, preincubation reduces binding.

### Example 5

### De-Immunized Fusions Show Improved Pharmacokinetics In Vivo

Pharmokinetics of fusions of Examples 2 and 3 were tested by administration to rats and mice to determine the serum half-life thereof in vivo. The fusions were administered at various concentrations by IP or IV injection; while unfused sdAbs were administered similarly to control rats. 100 mg of the fusions were injected i.p. into Wistar female rats. Plasma samples were obtained from injected rats at regular intervals of 5, 30, 60, 120, and 360 minutes, 24 hours, 48 hours, and 72 hours, and assayed for concentration of the fusion or unfused sdAB by ELISA. Briefly, corresponding antigens were immobilized in 384 well-plates (80 ng/well) overnight at 4° C. After 2 h blocking with soy milk, recombinant antibody fragments were titrated in duplicates and incubated for 1 h at RT. Detection was performed with mouse HRP-conjugated anti-HA antibody (Roche) using ABTS substrate. Absorbance was measured at 450 nm in an ELISA-reader. The plasma concentration is obtained for each time point and fitted to a two-compartment elimination model. Data were normalized considering maximal concentration at the first time point (5 minutes).

The pharmacokinetic studies compare the serum half-lives of PEP variant-sdAb fusions to unfused sdAbs in vivo. The fusions show increased serum half-lives at one or more concentrations. That is, in vivo serum half-life of a fusion according to the invention shows several fold improvement, e.g., by 200-500% (i.e., by 2 to 10 fold), compared to the unfused sdAb of interest. Results are illustrated in FIG. 3B.

### Example 6

### De-Immunized Fusions Retain Antigen Binding Spcificity

Binding of fusions of Examples 2 and 3 were analyzed for binding specificity of sdAbs portions to their respective antigens using ELISAs. Binding ELISAs were performed as described above. Briefly, the antigen for a given sdAb, at 80 ng/well, was immobilized overnight in 96 well-plates at 4° C. After 2h blocking with PBS/3% soy milk, recombinant antibody fragments (sdAbs) and fusions thereof to PEP variants were incubated for 1 h at room temperature. After washing the wells with PBS, anti-HA-HRP mAb (Roche) was used for detection. Optical density at 405 nm was measured and assays were performed in triplicate.

The binding studies compare binding of PEP variant-sdAb fusions with unfused sdAbs to the antigen corresponding to a given sdAb. Each fusion maintains specific binding to its respective antigen at one or more concentrations. That is, antigen binding by a fusion according to the invention shows approximately the same binding specificity compared to the unfused sdAb.

### Example 7

### De-Immunizaiton of VL18-3L-VL11 and VL18-3L-VL11-PEP

De-immunization of a specific PEP construct, the anti-TNF-alpha polypeptide VL18-3L-VL11-PEP (a VL dimer fusion with PEP), is described in detail below.

An in silico assessment (Alogonomics Epibase™-LONZA) against human T cell epitopes was carried out as a de-immunizing strategy of VL18-3L-VL11-PEP to reduce T_{H} epitope content. 3D models of VL18-3L-VL11-PEP were developed using Tripole modelling tools (LONZA).

Based on the results of the profiling and positioning of putative T-cell epitopes, a list of amino acid substitutions was proposed:

### Proposed Amino Acid Substitutions

In the summary listing below, the double mutants are linked by a hyphen, e.g. A51V-L54R, and variant substitutions involving the same position is given within brackets, separated by a slash, e.g. (A51V-L54R/A51V-L54E). In addition, two solvent-exposed framework cysteine to serine mutations were proposed to increase stability.

In Pep: E12D, T29H-K35D, and A45D (4 proposed amino acid substitutions, where the numbering of the substitutions refer to amino acid positions in SEQ ID NO:1 (PEP)).

In VL18: T7Q, V15P, (A51V-L54R/A51V-L54E), K63S, E79K, (C80S), T91A, L111K (9 proposed amino acid substitutions, where the numbering of the substitutions refers to amino acid positions in. SEQ ID NO:2).

In VL11: T7Q, V15P, R31S, (A51V-54R /A51V-L54E), K63S, E79K, (C80S), T91A, A100S, and E106K (11 proposed amino acid substitutions, where the numbering of the substitutions refer to amino acid positions in SEQ ID NO:3).

Sequences of VL18 and VL11 containing the substitutions are presented in FIGS. 2A-B, respectively. Sequences for the PEP domain is provided in FIG. 1, as described above. The figures show Kabat and Ordinal numbering for the various domains. CDRs are indicated by x. The proposed positions for substitutions are highlighted in gray. The mode of binding between VL18-3L-VL11-PEP and human albumin is illustrated in FIG. 3.

### Example 8

### Synthesis of De-Immunized VL18-3L-VL11-PEP Fusions

As a result of the proposed amino acid substitutions of Example 7, putative de-immunized variants of VL18-3L-VL11-PEP were synthesized, as shown below in Tables 1 and 2 below. A sequence encoding the HA-tag sequence (YPYDVPDYA) was also added at the C-terminus. Genes encoding the de-immunized variants were cloned into the pET28a expression vector by NheI and XhoI digestion.

TABLE 1

| | | Number of |
|---|---|---|
| Variant | | substitutions |
| identification | Domain substitutions | introduced |
| VL18-3L-VL11 | VL18: WT | — |
| PEP #6 | VL11: WT | |
| | PEP: WT | |
| VL18-3L-VL11 | VL18: WT | 4 aa substitutions |
| PEP DI #7 | VL11: WT | |
| | PEP: E12D, T29H-K35D, A45D | |
| VL18-3L-VL11 | VL18: T7Q, V15P, | 21 aa substitutions |
| DI3-PEP DI #8 | (A51V-L54R), K63S, E79K, | CDR3 x |
| | (C80S), T91A, L111K | |
| | VL11: T7Q, V15P, R31S, | |
| | (A51V-L54R), K63S, | |
| | E79K, (C80S), T91A, | |
| | A100S, E106K | |
| | PEP: E12D, T29H-K35D, A45D | |
| VL18-3L-VL11 | VL18: T7Q, V15P, K63S, | 17 aa substitutions |
| DI5-PEP DI #9 | E79K, L111K | CDR2 x; CDR3 x |
| | VL11: T7Q, V15P, R31S, | |
| | K63S, E79K, (C80S), | |
| | E106K | |
| | PEP: E12D, T29H-K35D, A45D | |

TABLE 2

Variant number

| Domain | Regions | Substitutions | 7 | 8 | 9 |
|---|---|---|---|---|---|
| VL18 | FwR1 | T7Q | | X | X |
| | | V15P | | X | X |
| | CDR2 | A51V-L54E | | | |
| | | A51V-L54R | | X | |
| | FwR3 | K63S | | X | X |
| | | E79K | | X | X |
| | CDR3 | T91A | | | |
| | FwR4 | L111K | | X | X |
| VL11 | FwR1 | T7Q | | X | X |
| | | V15P | | X | X |
| | CDR1 | R31S | | X | X |
| | CDR2 | A51V-L54E | | | |
| | | A51V-L54R | | X | |
| | FwR3 | K63S | | X | X |
| | | E79K | | X | X |
| | CDR3 | T91A | | | |
| | | A100S | | | |
| | FwR4 | E106K | | X | X |
| PEP | | E12D | X | X | X |
| | | T29H-K35D | X | X | X |
| | | A45D | X | X | X |
| Extra | | C80S | | X | X |
| | | C210S | | X | X |

The de-immunized variants were tested in expression studies, antigen cross-reactivity, affinity measurements, and efficacy studies in the cytotoxicity assay with L929 cell line as described in the Examples above. Results for a representative number of variants are presented below in Table 3.

TABLE 3

| | Expression | | | Efficacy | Efficacy |
|---|---|---|---|---|---|
| Variant | Yields | Antigen | Affinity | Cell | RA rat |
| # | (mg/L) | Cross-Reactivity | (Biacore) | Assay | model |
| VL18-3L-VL11 | 12-15 | Human and rat | 0.8 nM (human | High | High |
| PEP #6 | | TNF (VL18-3L- | TNF) | | |
| | | VL11) | | | |
| | | Human, rat, and | 4.7 nM (human | | |
| | | mouse Albumin | albumin) | | |
| | | (Pep) | | | |
| | | | 0.4 nM (rat | | |
| | | | albumin | | |
| | | | 42.1 nM (mouse | | |
| | | | albumin) | | |
| VL18-3L-VL11 | 12-15 | Human and rat | 0.7 nM (human | High | High |
| PEP DI #7 | | TNF (VL18-3L- | TNF) | | |
| | | VL11) | | | |
| | | Human, rat, and | 61.8 nM | | |
| | | mouse Albumin | (human | | |
| | | (Pep DI) | albumin) | | |
| | | | 28.1 nM (rat | | |
| | | | albumin) | | |
| | | | NC (mouse | | |
| | | | albumin) | | |
| VL18-3L-VL11 | 6-8 | Human and rat | 0.6 nM (human | High | High |
| DI3-PEP DI #8 | | TNF (VL18-3L- | TNF) | | |
| | | VL11-DI3) | | | |
| | | Human, rat, and | 88.5 nM | | |
| | | mouse Albumin | (human | | |
| | | (Pep DI) | albumin) | | |
| | | | 22.4 nM (rat | | |
| | | | albumin) | | |
| | | | NC (mouse | | |
| | | | albumin) | | |
| VL18-3L-VL11 | 12-15 | Human and rat | 0.4 nM (human | High | High |
| DI5-PEP DI #9 | | TNF (VL18-3L- | TNF) | | |
| | | VL11DI5) | | | |
| | | Human, rat, and | 92 nM (human | | |
| | | mouse Albumin | albumin) | | |
| | | (Pep DI) | | | |
| | | | 32 nM (human | | |
| | | | albumin) | | |
| | | | NC (mouse | | |
| | | | albumin) | | |

### Example 9

### Large Scale Expression of De-Immunized VL Fusions with PEP Variants

An expression construct designed to express a recombinant de-immunized fusion of the invention (without His Tag) in bacteria was used to transform E. coli Tuner (DE3), E. coli BLR (DE3), and/or DL21 (DE3) to generate clones for expression screening. Induction experiments (0.1 mM IPTG, 18 hrs) were performed on three colonies. SDS-PAGE or immunoblot were run to compare protein expression (total and soluble) and the best expresser banked and then utilized for production first at the 15L scale followed by a scale-up production at 100L. Growth and induction were performed as follows. Briefly, cells from a fresh culture are grown at 37° C. in SB medium plus a selection agent to an OD between 0.7 and 0.9, the temperature was dropped to 18° C., and the culture induced with 0.1 mM IPTG. After an overnight exposure, the cell paste was harvested by centrifugation and stored at −20° C. until purification. Either an SDS-PAGE or a Western-immunoblot (using an antigen specific antibody) was performed on an analytical sample to confirm protein expression. Experiments were conducted in conjunction with Paragon (USA).

Results of Coomassie Blue SDS-PAGE expression analysis of the recombinant de-immunized fusion VL18-3L-VL11 D13-PEP DI #8 are illustrated in FIG. 4. The position in the gel of the expressed fusion is indicated by the arrow. Expression yields for for de-immunized PEP variant fusions are provided in Table 4 below.

TABLE 4

| Clone # | Induction time | Yield (mg/L) |
|---|---|---|
| VL18-3L-VL11-PEP | ON/18° C. | 12-15 |
| VL18-3L-VL11-PEP DI | ON/18° C. | 12-15 |
| VL18-3L-VL11-DI#3-PEP DI | ON/18^{a} C. | 6-8 |
| VL18-3L-VL11-DI#5-PEP DI | ON/18° C. | 12-15 |

### Example 10

### Purification of De-Immunized VL Fusions with PEP Variants

The recombinantly expressed de-immunized fusions of Example 9 were purified using Protein L and/or human albumin affinity chromatography standard procedures. Briefly, cell paste was resuspended in PBS buffer supplemented with anti-proteases using a polytron at medium speed. Suspension was lysed using a homogenizer, and soluble material clarified by centrifugation. Clarified lysate was loaded onto a chromatography column containing Protein L and/or human albumin resin. The resin was washed to remove contaminating proteins and endotoxin, and the bound polypeptide eluted using 100 mN Glycine (pH=3). Fractions containing the fusion of interest were pooled and processed for residual endotoxin removal (ActiClean Etox).

Additional polishing steps may be performed to meet desired purity specifications, with the type of polishing step implemented (e.g. IEX, SEC) to be determined following analysis of the contaminants. Steps included SP Sepharose cation exchange; size exclusion chromatography, and endotoxin reduction reduction, followed by formulation. The downstream process development for the de-immunized fusion VL18-3L-VL11 DI3-PEP DI #8 is illustrated in FIGS. 5A-E. A schematic representation of the downstream process development is illustrated in FIG. 5A. Results of Coomassie Blue SDS-PAGE expression analysis following Protein L Affinity purification of the de-immunized fusion VL18-3L-VL11 DI3-PEP DI #8 is illustrated in FIGS. 5B-C. The results of Coomassie Blue SDS-PAGE expression analysis following SP Sheparose cation exchange chromatography of the de-immunized fusion VL18-3L-VL11 DI3-PEP DI #8 is illustrated in FIG. 5D. The results of Coomassie Blue SDS-PAGE expression analysis following size exclusion chromatography of the de-immunized fusion VL18-3L-VL11 DI3-PEP DI #8 is illustrated in FIG. 5E.

The final purified fusion was dialyzed into PBS buffer, concentrated to ˜5 mg/ml, and stored frozen at −80° C. All steps of the purification were monitored by SDS-PAGE-Coomassie and A280 absorbance. Purified polypeptides were accompanied with a specifications sheet documenting purity (SDS-PAGE-coomassie), yield (Bradford assay or A280), endotoxin (PTS EndoSafe assay) and identity (Western blot-antibody). Experiments were conducted in conjunction with Paragon (USA).

### Example 11a

### Therapeutic Effects of De-Immunized VL18-3L-VL11-PEP Variants

To determine therapeutic effects of de-immunized VL18-3L-VL11-PEP variants, an established rat-adjuvant induced arthritis model (AIA) was used. Freunds complete adjuvant (FCA) induces rheumatoid arthritis in rats. For example, an injection of FCA in the base of the tail results in chronic arthritis in the rat, involving multiple joints and promoting a widespread systemic disease, severe discomfort, and distress.

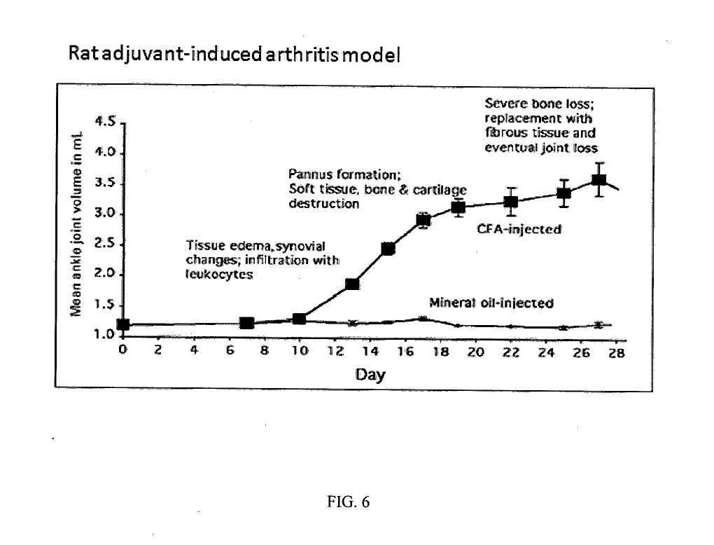

Wistar female rats (Charles River) with a mean bodyweight of 150-170 g, received an injection of FCA (1 mg/ml) intradermally (i.d.). The induced arthritis is assessed over 3 weeks following disease induction, based on the following: clinical scores (limb analysis), bodyweight, measurement of ankle joints, and histologically, as described below. All rats were bred and maintained under specific pathogen-free conditions at Institute of Medicine animal breeding facility according to institute guidelines. The animals were fed with standard rodent chow and water and maintained in ventilated cages. An adapted AIA model cartoon (adapted from In Vivo Models of Inflammation, Vol. I, C. S. Stevenson, L. A. Marshall, and D. W. Morgan (eds.), page 17 (2006) Birkhauser Veriag Basel, Switzerland) is illustrated in FIG. 6.

Wistar female rats (Charles River) received an injection of Freund's complete adjuvant (FCA) (1 mg/ml) intradermally (i.d.). The rats were treated with 100 μg VL18-3L-VL11 and VL18-3L-VL11-PEP, as well as two de-immunized variants of each (VL18-3L-VL11 DI #3 and VL18-3L-VL11 DI #5; VL18-3L-VL11 DI #3-PEP DI and VL18-3L-VL11 DI #5-PEP DI). VL18-3L-VL11 and the two de-immunized mutants were administered daily. VL18-3L-VL11-PEP and the two PEP fusion de-immunized variants were administered at 2 day intervals. Dexamethasone was used as a positive control. PBS was used as a negative control (animal were injected with the vehicle only).

The severity of arthritis was assessed after disease induction based on clinical scores (limb analysis), ankle joint measurement, and histological analysis, providing arthritis severity scores in rats (n=6) during a 20-day period of treatment.

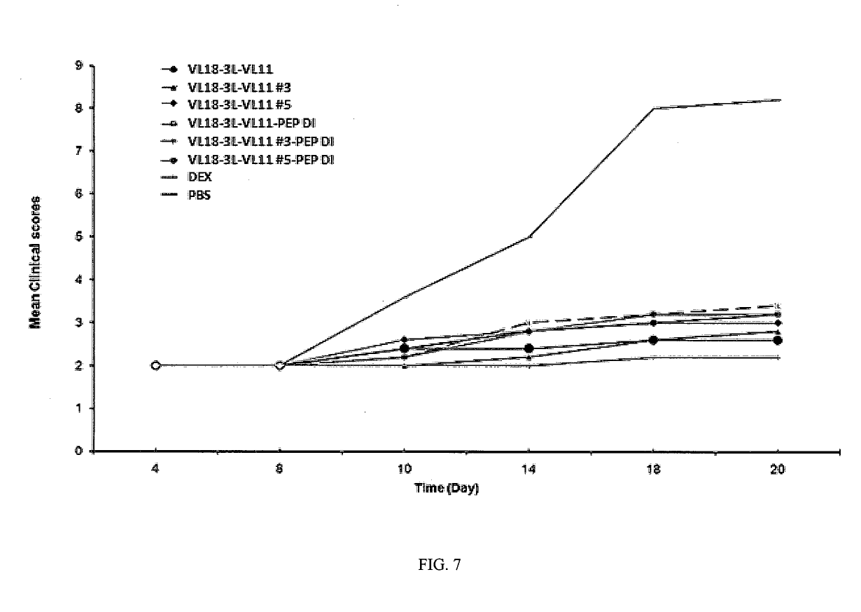

Evaluation was based on the total score of the four limbs of each animal. The severity of the arthritis was quantified at 2-4-day intervals by a clinical score measurement from 0 to 3 as follows: 0=normal; 1=slight erythema; 2=moderate erythema; 3=strong erythema to incapacitated limb; max score=12. As compared to the negative control (PBS), rats treated with de-immunized VL18-3L-VL11 and VL18-3L-VL11-PEP variants, like the ones treated with the wild-type VL18-3L-VL11/PEP and dexamethasone, revealed an obvious reduction in inflammation and joint destruction in the four limbs. Results are illustrated in FIG. 7.

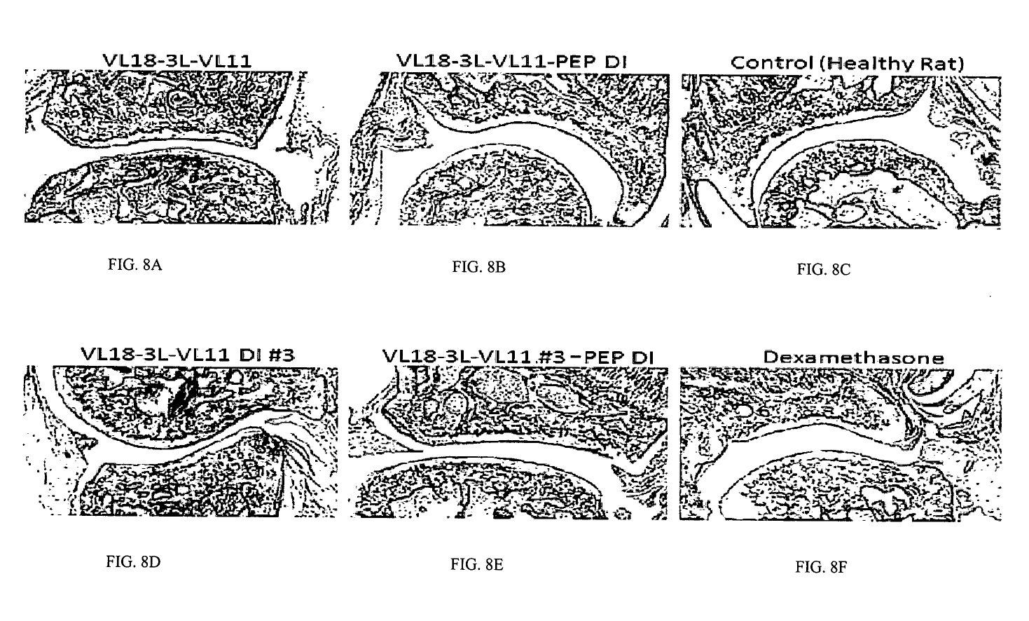

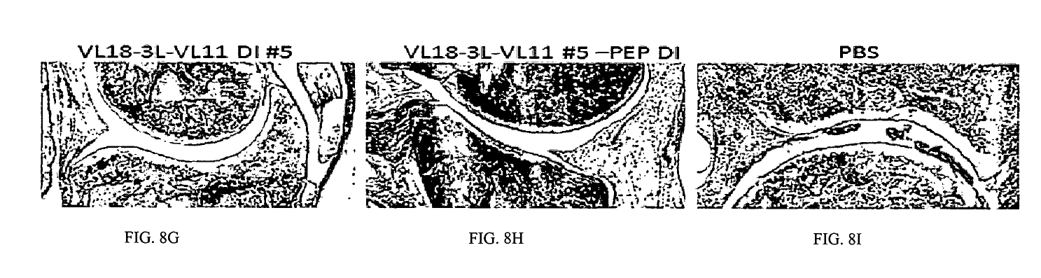

For histopathological observation, paw samples were collected at the time of sacrifice (day 20). Samples were fixed immediately in 10% neutral buffered formalin solution, after being fixed, samples also were decalcified in 10% formic acid and then dehydrated using increased ethanol concentrations (70%, 96%, and 100%). Samples were next embedded in paraffin, sectioned using Microtome (Leica RM 2145, Germany) and stained with hematoxylin and eosin for morphological examination. Images were acquired using a Leica DM 2500 (Leica microsystems, Germany) microscope equipped with a colour camera. Histograms were prepared from joints of AIA rats treated with de-immunized VL18-3L-VL11 and VL18-3L-VL11-PEP variants, like the ones treated with the wild-type VL18-3L-VL11/PEP and dexamethasone. Results are illustrated in FIGS. 8A-I.

### Example 11b

### Therapeutic Effects of De-Immunized VL18-3L-VL11/PEP Variants in Prevention of Chronic Polyarthriths

To determine efficacy effects of de-immunized VL18-3L-VL11-PEP variants in preventing the development of arthritis, a transgenic mouse (Tg197) model is used. The Tg197 model of arthritis is a humanized TNF transgenic mouse model with human TNF deregulated expression resulting in the spontaneous development of arthritis pathology closely resembling that of the human rheumatoid arthritis (Keffer et al. 1991).

The Tg197 mouse model develops chronic polyarthritis with 100% incidence at 4-7 weeks of age and provides a fast in vivo model for assessing human therapeutics for the treatment of rheumatoid arthritis. It was successfully used in establishing the therapeutic efficacy of Remicade™ and is currently used widely for efficacy studies testing biosimilars or novel anti-human TNF-alpha therapeutics.

The experimental study with Tg197 involves: 4 groups of 8 mice, including a negative control, a positive control (Remicade™) and test treated groups (VL18-3L-VL11-PEP variants) Another 4 mice are sacrificed at the beginning of the study to serve as controls for histopathology. A 7 week prophylactic treatment is performed from week 3 to week 10. Administration of the anti-TNF-α antibodies are made twice weekly at a final dose of 10 mg/kg by intraperitoneal injection. During the 7 week treatment period clinical scores are recorded by observing macroscopic changes in joint morphology for each animal. At 10 weeks of age, all animals are sacrified and joints and sera are collected. Sera are stored at −70° C. and ankle joints in formalin. Ankle joint sections are embedded in paraffin, sectioned and then subsequently used for histopathological evaluation of disease progression.

Example 12a

### Pharmacokinetics of De-Immunized VL Fusions with PEP Variants

To determine in vivo the plasma-time course and tissue distribution of fusions with PEP variants according to the invention, a pharmacokinetic study is performed in rats. Rats are administered a single SC/IV dose of ¹⁴C-labled de-immunized fusion with a VL-VL dimer. The radiolabled fusion polypeptide is prepared at the start date or one day prior to the start date; the radiochemical concentration is analyzed pre- and post-dose; and stability is evaluated over the dosing interval by radiochemical HPLC analysis. The study design is as outlined in Table 5 below:

TABLE 5

| | | Dose | Dose | Dose | |
|---|---|---|---|---|---|
| Group & | No. Animals | Volume | Conc. | Radioactivity | Samples |
| Route | & Sex | (mL/kg) | (mg/kg) | (μCi/kg) | Collected |
| 1 | 6M | TBD | TBD | ~100 | Blood, |
| SC/IV | (2 cohorts | | | | Plasma |
| | of 3) | | | | |
| 2 | 6M | TBD | TBD | ~100 | Tissues, |
| SC/IV | | | | | Residual |
| | | | | | Carcasses |

The rats are observed twice daily for mortality and signs of ill health or adverse reaction to the treatment. For Group 1, samples are collected from cohort bleeds as follows: about 0.5 mL of blood or plasma is collected from three animals per time point, including pre-dose, and 1, 2, 4, 8, 12, 24, 48, 72, 96, 120 and 168 hours post-dose. For Group 2, samples are collected as follows: tissues are collected from one animal per time point, at 1, 4, 8, 24, 72 and 168 hours post-dose. Tissues included are: adrenal gland, bladder (urinary), bone, bone marrow, brain, eyes (both), fat (brown), fat (white), heart, kidneys, large intestine/cecum, liver, lung, lymph nodes (mesenteric), muscle, pancreas, prostate, salivary glands, skin, small intestine, spleen, stomach, testes, thymus, thyroid, and residual carcass.

Radioactivity levels in plasma, tissues, and residual carcasses is measured in accordance with known procedures. Aliquots of tissue homogenates are oxidized or solubilized before analysis, while other samples are analyzed directly by mixing aliquots with scintillation fluid. Total recovery of dosed radioactivity and recovery at each interval is determined for samples from tissues and carcasses. Tissue distribution is analyzed via individual tissues obtained at necropsy. A pharmacokinetic plasma curve is generated to provide in vivo plasma-time course and tissue distribution of de-immunized fusions of the invention.

### Example 12b

### Biodistribution of De-Immunized VL Fusions with PEP Variants

To determine in vivo the plasma-time course and tissue distribution of fusions with PEP variants according to the invention, a preliminary biodistribution assay was performed in rats, using ^{99m}Tc(CO₃)-labelling, as follows:

Preparation of ^{99m}Tc(I) Tricarbonyl Precursor

Into a vial of IsoLin Kit (Covidien), ^{99m}TcO₄⁻/saline (2 ml, ˜25 mCi) was added. The mixture was heated for 30 minutes and the pH of the resultant solution was adjusted to 7.4. The radiochemical purity of [^{99m}Tc(CO)₃(H₂O)₃]⁺ was checked by RP-HPLC and instant thin-layer chromatography (ITLC) using 6 N HCl (5%) in MeOH as eluent (R_{f}≈1). Labeling of His₆-VL18-3L-VL11 and His₆-VL18-3L-VL11-PEP

All constructs were buffered with 50 mM NaH₂PO₄, 300 mM NaCl pH 6.0 and concentrated to ˜0.5 mg/ml. Compoundsfac-[^{99m}Tc(CO)₃]-His₆-VL18-3L-VL11 and fac-[^{99m}Tc(CO)₃]-His₆-VL18-3L-VL11-PEP, were obtained in 0.125 mg/mL final concentration, by reacting the constructs withfac-[^{99m}Tc(CO)₃(H₂O)₃]⁺. Briefly, a solution offac-[^{99m}Tc(CO)₃(H₂O)₃]⁺ containing 2% of SDS surfactant was added to a microcentrifuge tube containing the constructs. The mixture reacted at 37° C. for 1 hour and the radiochemical purity of the ⁹⁹mTc-radiolabeled constructs was checked by ITLC (R_{f}=0; radiochemical yield ˜80-95%). Unreacted free “^{99m}Tc(CO)₃” was removed by desalting using a Sephadex G-25 column eluted with 50 mM NaH₂PO₄, 300 mM NaCl pH 6.0.

### Partition Coefficient Determination

The partition coefficient was evaluated by the “shake-flask” method. The radioactive conostructs were added to a mixture of octanol (1 mL) and 0.1 M PBS pH 7.4 (1 mL), which had been previously saturated with each other by stirring. This mixture was vortexed and centrifuged (3000 rpm, 10 min) to allow phase separation.

Aliquots of both octanol and PBS were counted in a y-counter. The partition coefficient (P_{o/w}) was calculated by dividing the counts in the octanol phase by those in the buffer, and the results were expressed as log P_{o/w}±SD.

### In Vitro Stability Determination

The ^{99m}Tc-radiolabeled constructs were stored in buffer solution at 37° C. for 24 h. After incubation, the solutions were analyzed by ITLC. No release of [^{99m}Tc(CO)₃(H₂O)₃]⁺ was observed.

### Biodistribution Studies

In vivo evaluation studies of radiolabeled constructs were performed in Wistar female rats (Charles River) at 15 min, 1 h, 3 h, 6 h, and 24 h. All animal experiments were performed in accordance with the guidelines of the institutional animal ethics committee. Animals were injected intraperitoneally under light isofluorane anaesthesia with the radiolabeled compounds (˜1 mCi; ˜300 μL; ˜16-20 μg of ^{99m}Tc(CO)₃-labeled constructs) and sacrificed by excess anaesthesia.

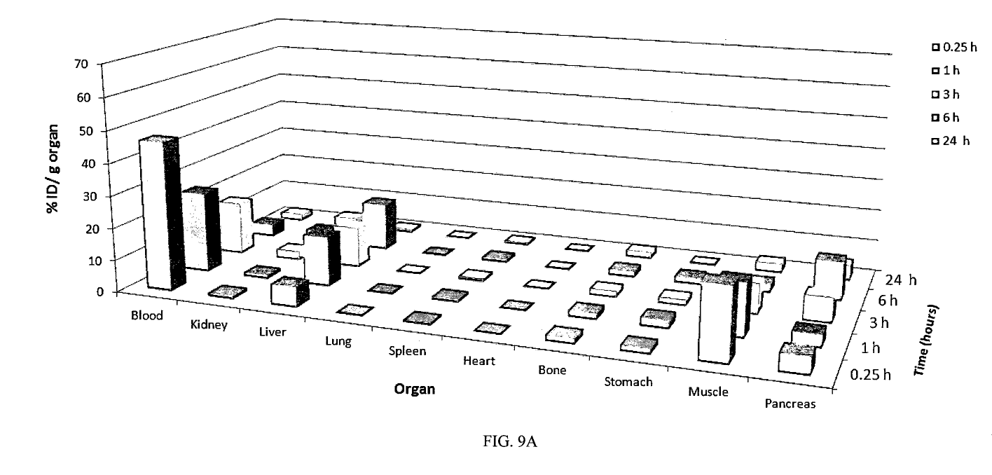

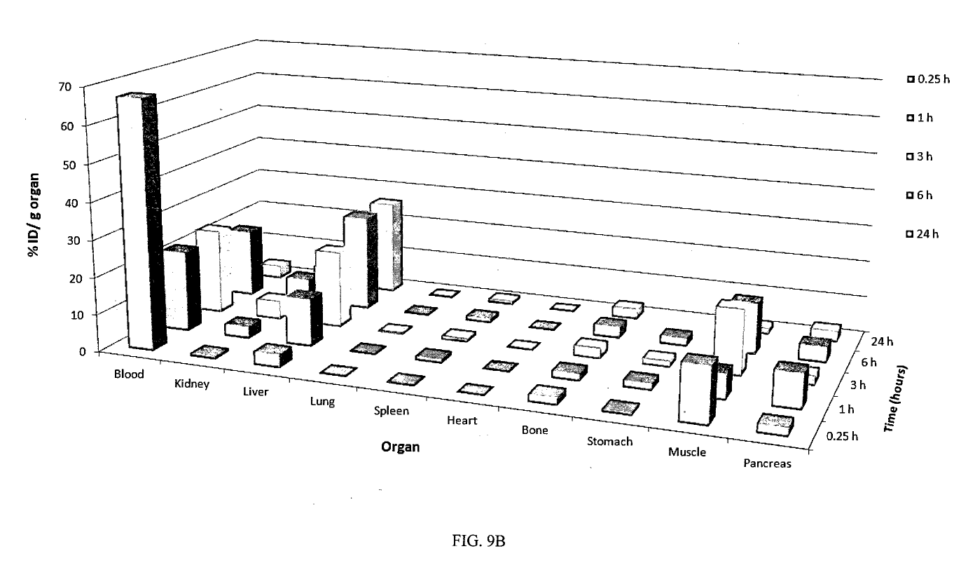

The radioactivity in the sacrificed animals was measured using a dose calibrator (Curiemeter IGC-3, Aloka, Tokyo, Japan or Carpintec CRC-15W, Ramsey, USA). The difference between the radioactivity in the injected animals and that in the killed animals was assumed to be due to excretion. Tissues of interest were dissected, rinsed to remove excess blood, weighed, and their radioactivity measured using a y-counter (LB2111, Berthold, Germany). The uptake for most relevant organs, including each of blood, bone, and muscle tissues, was estimated assuming that these organs constitute 6, 10, and 40% of the total body weight of the animal, respectively. Results were expressed as % ID/Organ for 15 min, 1 h, 3 h, 6 h, and 24 h after i.p. administration in Wistar rats (n=3). Urine also was collected and pooled together at the time the animals were killed. Results are illustrated in FIGS. 9A-B for fac-[^{99m}Tc(CO)₃]-VL18-3L-VL11 (FIG. 9A) and fac-[^{99m} Tc(CO)₃]-VL18-3L-VL11-PEP (FIG. 9B).

### Example 13

### Toxicity Study of De-Immunized VL Fusions with PEP Variants

To determine toxicity of the fusion of de-immunized PEP variants of the invention, the study design outlined in Table 6 below is implemented in rats.

TABLE 6

Dose Escalation Study

| Phase A | Males | Females |
|---|---|---|
| Dose Level 1 | 2 | 2 |
| Dose Level 2 | 2 | 2 |
| Dose Level 3 | 2 | 2 |
| Dose Level 4 | 2 | 2 |

| | MTD Study | |
|---|---|---|
| Phase B | Males | Females |
| Control | 5 | 5 |
| High Dose | 5 | 5 |

In Phase A, all doses are administered on day 1 to determine the maximum tolerated dose (MTD); animals are dosed once at each designated level. In Phase B, animals are dosed once on day 1 at a dose based on Phase A. Administration is by subcutaneous or intravenous injection.

Animals in Phase A are observed twice daily for mortality and/or moribundity for 4 days at each level. Animals in Phase B are observed twice daily for 7 days. Also, body weight is measured for each animal at each observation time point. For Phase B animals, clinical pathology analyses are performed including: hematology, coagulation, clinical chemistry, and urinalysis evaluations on surviving animals at termination of the study, and tissues are saved from non-surviving animals. The following organs also are weighed following termination of Phase B animals: adrenals, brain, heart, kidneys, liver, spleen, thyroid with parathyroid, thymus. Standard statistical analysis is used.

### Example 14

### Acute Toxicity Study of De-Immunized Fusions with PEP Variants

To determine toxicity of the de-immunized fusions of the invention, the study design outlined in Table 7 below is implemented in rats.

TABLE 7

| | Males | Females |
|---|---|---|
| Vehicle Control | 1 | 1 |
| Low Dose | 1 | 1 |
| Mid Dose | 1 | 1 |
| High Dose | 1 | 1 |

Doses are administered by subcutaneous injection once on day 1 of the study, and the animals are observed twice daily for mortality and moribundity. Electrocardiograms also are obtained for all animals prior to the initiation of the study, as well as pre- and post-dose on study days 1 and 14. Detailed clinical observations and body weights also are measured daily during the course of the study. Clinical pathology analyses also are conducted, including hematology, coagulation, clinical chemistry, and urinalysis evaluations on surviving animals at termination of the study (day 15).

### Example 15

### 4-Week Toxicity Study of De-Immunized Fusions with PEP Variants

To further assess the toxicity of the de-immunized fusions of the invention, the study design outlined in Table 8 below is implemented in rats.

TABLE 8

| | Main Study | | Recovery | | Toxicokinetics | |
|---|---|---|---|---|---|---|
| | Males** | Females** | Males | Females | Males | Females |
| Vehicle | 15 | 15 | 5 | 5 | — | — |
| Control | | | | | | |
| Low Dose | 15 | 15 | — | — | 6 | 6 |
| Mid Dose | 15 | 15 | — | — | 6 | 6 |
| High Dose | 15 | 15 | 5 | 5 | 6 | 6 |

Six animals per sex per group are designated for neurobehavioral evaluations and four animals per sex per group are designated for respiratory evaluations.

Doses are administered by subcutaneous or intravenous injection weekly (days 1, 8, 15, 22, and 29 of the study), and the animals are observed twice daily for mortality and moribundity. Detailed clinical observations and body weights also are measured weekly during the course of the study. Six animals of the main study per sex per group undergo neurobehavioral evaluations prior to dosing, at the estimated time of peak effect on day 1, and 24 hours following the first dose.

Four main study animals per sex per group are subjected to respiratory evaluations. The animals are placed in a plethysmograph chamber at least 2 hours prior to dosing on day 1. After at least 60 minutes, respiratory monitoring is initiated to establish baseline data. The animals are temporarily removed from the plethysmograph chambers for dosing after at least 1 hour of baseline recording. Immediately following dosing, the animals are returned to the plethysmograph chamber and continue to be monitored for a period of at least 4 hours. Food and water are not available during respiratory recording sessions. Respiratory endpoints measured include respiratory rate, tidal volume, and minute volume.

Clinical pathology analyses also are conducted, including hematology, coagulation, clinical chemistry, and urinalysis evaluations on surviving main study animals once at the terminal or recovery necropsy. Ophthalmology examination also is conducted in all animals before the study and in surviving main study animals at termination and recovery.

Toxicokinetics are measured based on blood (0.5 mL) collected on days 1 and 29 (from cohorts of 3 animals/sex; 2 cohorts bled three times to equal six time points and 216 total samples). Blood samples collected from the main study animals also are pre-tested for immunogenicity and immunophenotyping, and tested once again at the terminal or recovery necropsy (280 total samples). All main study (day 31) and recovery (day 57) animals are subjected to necropsy. Animals from the toxicokinetics study are euthanized and discarded. Weights are obtained for the following organs: adrenals, brain, heart, kidneys, liver, lungs, ovaries with oviducts, pituitary, prostate, salivary glands, seminal vesicles, spleen, thyroid with parathyroid, thymus, testes, and uterus.

Tissues are analyzed by microscopic pathology for all animals in the vehicle control and high dose groups and all found-dead animals. A full set of standard tissues (approximately 65), including target organs, are analyzed by microscopic pathology in low and mid dose groups and all recovery animals. Any gross lesions also are analyzed by microscopic pathology for any animal exhibiting same.

Data are analyzed by standard statistical analysis. Group pair-wise comparisons also are used for continuous endpoints; the Cochran Mantel Haenszel Test is used for categorical neurobehavioral endpoints only; repeated measures analysis of covariance is used with respiratory endpoints only. Toxicokinetic analysis include standard parameters such as AUC, t_{1/2}, tmax, and Cmax.

### Example 16

### Cardiovascular Safety of De-Immunized Fusions with PEP Variants

To determine toxicity and cardiovascular safety of the de-immunized fusions with PEP variants of the invention, the study design outlined in Table 9 below is implemented in monkeys.

TABLE 9

| | |
|---|---|
| Main Study | Recovery |

| | Males* | Females* | Males | Females |
|---|---|---|---|---|
| Placebo | 5 | 5 | 2 | 2 |
| Low Dose | 5 | 5 | — | — |
| Mid Dose | 5 | 5 | — | — |
| High Dose | 5 | 5 | 2 | 2 |

*Two animals/sex/group designated for cardiovascular evaluation

Two animals per sex per group are designated for cardiovascular evaluation. Doses are administered by subcutaneous or intravenouos injection weekly (days 1, 8, 15, 22, and 29 of the study), and the animals are observed twice daily for mortality and moribundity. Detailed clinical observations and body weights also are measured weekly during the course of the study. Animals designated for cardiovascular evaluation are observed via remote camera on day 1. Physical examinations of the animals are conducted by a veterinarian on all animals prior to initiation of the study. Ophthalmology examination also is conducted in all animals before the study and in surviving main study animals at termination and recovery.

For cardiovascular evaluations, two main study animals per sex per group are surgically implanted with a pressure transducer equipped telemetry transmitter. The transmitter assembly is secured internally, the fluid-filled catheter placed into an appropriate artery, and ECG leads placed to allow for collection of cardiovascular (hemodynamic and electrocardiographic) data. For those animals designated for cardiovascular evaluations, data are collected while the animals are allowed free movement in the home cage. The animals are monitored continuously for at least 2 hours prior to, and approximately 20 hours subsequent to, the first administration. The following parameters are monitored: systolic, diastolic and mean arterial blood pressures; heart rate; electrocardiogram (RR, PR, QRS, QT, and QTc); and body temperature.

All animals are subjected to electrocardiogram testing before the study and all surviving animals pre-dose and post-dose on Day 1, pre-dose and post-dose prior to the terminal necropsy, and prior to the recovery necropsy. For those animals designated for cardiovascular evaluations, representative ECG tracings are printed from the raw data telemetry record and these animals have additional ECG tracings printed at the expected time of peak effect on day 1 and at the end of the cardiovascular monitoring period. All traces are reviewed by a board certified veterinary cardiologist who performs a qualitative evaluation of the electrocardiograms.

Clinical pathology analyses also are conducted, including hematology, coagulation, clinical chemistry, and urinalysis evaluations on all animals prior to surgery, pre-test, and all surviving animals prior to the terminal or recovery necropsies. Toxicokinetics are measured based on blood collected on days 1 and 29 at six time points from each animal not designated for cardiovascular evaluation (384 total samples).

Blood samples collected from all animals not designated for cardiovascular evaluation also are pre-tested for immunogenicity and immunophenotyping, and tested again at the terminal and recovery necropsies (72 total samples). All animals not designated for cardiovascular evaluation are subjected to necropsy on day 31 or 57.

Weights are obtained for the following organs: adrenals, brain, heart, kidneys, liver, lungs, ovaries with oviducts, pituitary, prostate, salivary glands, spleen, thyroid with parathyroid, thymus, testes, and uterus. Tissues are analyzed by microscopic pathology for all animals not designated for cardiovascular evaluation. A full set of standard tissues (approximately 70) are analyzed and any gross lesions also are analyzed by microscopic pathology for any animal exhibiting same.

Data are analyzed by standard statistical analysis. Repeated measures analysis of covariance is used with telemetry data only. Toxicokinetic analysis include standard parameters such as AUC, t_{1/2}, tmax, and Cmax.

### Example 17

### 26-Week Toxicity Study of De-Immunized Fusions with PEP Variants

To further assess the toxicity of the de-immunized fusions with PEP variants of the invention, the study design outlined in Table 10 below is implemented in rats.

TABLE 10

| | |
|---|---|
| Main Study | Toxicokinetics |

| | Males | Females | Males | Females |
|---|---|---|---|---|
| Vehicle Control | 15 | 15 | — | — |
| Low Dose | 15 | 15 | 6 | 6 |
| Mid Dose | 15 | 15 | 6 | 6 |
| High Dose | 15 | 15 | 6 | 6 |

Doses are administered by subcutaneous or intravenouos injection weekly (27 total doses; final dose on day 183 of the study), and the animals are observed twice daily for mortality and moribundity. Detailed clinical observations and body weights also are measured weekly during the course of the study. Ophthalmology examination also is conducted in all animals before the study and in surviving main study animals at termination.

Clinical pathology analyses also are conducted, including hematology, coagulation, clinical chemistry, and urinalysis evaluations on surviving main study animals once at the terminal necropsy.

Toxicokinetics are measured based on blood (0.5 mL) collected on days 1 and 183 (from cohorts of 3 animals/sex; 2 cohorts bled three times to equal six time points and 216 total samples). Blood samples collected from the main study animals also are pre-tested for immunogenicity and immunophenotyping, and tested once again at the terminal necropsy (240 total samples). All main study animals (on day 185) are subjected to necropsy. Animals from the toxicokinetics study are euthanized and discarded. Weights are obtained for the following organs: adrenals, brain, heart, kidneys, liver, lungs, ovaries with oviducts, pituitary, prostate, salivary glands, seminal vesicles, spleen, thyroid with parathyroid, thymus, testes, and uterus.

Tissues are analyzed by microscopic pathology for all animals in the vehicle control and high dose groups and all found-dead animals. A full set of standard tissues (approximately 65), including target organs, are analyzed by microscopic pathology in low and mid dose groups. Any gross lesions also are analyzed by microscopic pathology for any animal exhibiting same.

Data are analyzed by standard statistical analysis. Toxicokinetic analysis include standard parameters such as AUC, t_{1/2}, tmax, and Cmax.

### Example 18

### Clinical Development of De-Immunized Fusions with PEP Variants

A preliminary Phase ½ clinical development plan for the anti-TNF-alpha polypeptides of the invention involves three stages, as outlined below.

Stage 1 involves a Phase 1a single ascending dose (SAD) safety and pharmacokinetic (PK) study in normal volunteers, followed by a Phase 1b multiple ascending dose (MAD) study in patients with moderate to severe rheumatoid arthritis. The Phase 1 a study is designed as a single-center, sequential-cohort, double-blind, placebo-controlled, SAD study in 40 healthy volunteers aged 18-55 years, inclusive. Healthy adult volunteers are selected who have had no prior exposure to therapeutic antibodies (investigational or other). The study evaluates the safety and tolerability, and characterizes the PK and pharmacodynamics (PD), of the anti-TNF-alpha polypeptides of the invention, following successively higher single doses. Dose selection is based on extrapolation from a pharmacologically active dose.

Eligible adult subjects are assigned sequentially to 1 of 5 cohorts, at successively higher single doses. Eight subjects per cohort are randomized in a 3:1 manner to receive active drug or matching placebo. Subjects are confined in a Phase 1 unit for 12 hours prior to dosing, during dosing, and for 24 hours after dosing (Study Days 1-2) for observation and PK/PD sampling. Subjects return on Study Day 8 (7 days after dosing) and Study Day 29 for additional safety evaluations and at more frequent intervals for PK sampling. The safety and available PK data from all subjects are reviewed after all subjects in a cohort have completed the Study Day 8 evaluation. Since PD can be assessed in normal volunteers, the study can confirm the PK/PD simulation derived from data from rat and single dose monkey studies.

Stage 2 involves a Phase lb study designed as a multi-center, sequential-cohort, double-blind, placebo-controlled, MAD study in 40 to 50 subjects with moderate to severe rheumatoid arthritis (based on the American College or Rheumatology Criteria of 1987 and 2010 Classification Systems). The study evaluates the safety and tolerability, and characterizes the PK, PD, and preliminary clinical efficacy (based on ACR20/DAS28 at Week 14) of the anti-TNF-alpha polypeptides of the invention in subjects with rheumatoid arthritis following 4 doses administered subcutaneously.

Eligible adult subjects, who continue to receive methotrexate at a weekly stable dose, are assigned sequentially to 1 of up to 5 cohorts, at successively higher multiple doses. Eight to ten (8-10) subjects per cohort are randomized in a 3:1 (or 4:1) manner to receive active drug or matching placebo on Study Days 1, 29/Week4, 57/Week 8, and 85/Week 12. Subjects return at specified time points for safety and PK evaluations, and at Weeks 6 and 14 for efficacy evaluations. The safety and PK data from all subjects are reviewed after all cohort subjects complete the Week 6 evaluation. Eligible subjects meet the following criteria: (i) diagnosis of rheumatoid arthritis within 3 months; and (ii) treatment for at least 12 weeks with methotrexate prior to randomization, but do not have any of the following characteristics: (i) an autoimmune disease other than rheumatoid arthritis; (ii) a history of acute inflammatory joint disease other than rheumatoid arthritis; (iii) latent or active tuberculosis; (iv) a fever or persistent chronic or active recurring infection requiring treatment with antibiotics, antivirals, or antifungals within 4 weeks prior to the screening visit, or history of frequent recurrent infections; (v) immunization with any live (attenuated) vaccine within 3 months prior to the randomization visit (e.g., varicella-zoster vaccine, oral polio, rabies); (vi) tuberculosis vaccination within 12 months prior to screening; or (vii) prior therapy with a TNF inhibitor or any other biologic agents within 3 months prior to inclusion.

Stage 3 invovles an initial Phase 2 study designed as a multi-center, randomized, double-blind, placebo-controlled, parallel arm study of 3 dose regimens of the anti-TNF-alpha polypeptides of the invention, administered subcutaneously, in approximately 200 subjects with moderate to severe rheumatoid arthritis (ACR 1987 and 2010 Classification Systems). The study evaluates the safety, tolerability, PD, and preliminary efficacy (based on ACR20 and/or DAS28at Weeks 14 and 26) of the anti-TNF-alpha polypeptides of the invention at 3 dose regimens over 6 months, identifying safe and therapeutic doses for Phase 3 trials.

Eligible adult subjects, who continue to receive methotrexate at a weekly stable dose, are randomized in a 3:1 manner to receive 1 of 3 dose regimens of an anti-TNF-alpha polypeptide of the invention or placebo. DMARDs other than stable dosages of methotrexate are stopped at least 4 weeks, and 12 weeks for biologics, prior to randomization. Subjects receive methotrexate weekly for at least 12 weeks (stable dosage for at least 4 weeks) before enrollment. Stable dosages of nonsteroidal anti-inflammatory drugs (NSAIDs) are permitted.

Approximately 200 eligible subjects are randomized to receive active drug or matching placebo every 4 weeks for 7 doses. Subjects return at specified time points for safety and PK evaluations, and at Weeks 14 and 26 for efficacy evaluations. The last study visit will be at Week 28.

Eligible subjects meet the same criteria as outlined above, except that there is a diagnosis of rheumatoid arthritis of 3 months duration.

### Example 19

### Immunogenicity analysis of de-immunized VL18-3L-VL11-PEP variants

Immunogenicity analysis of de-immunized VL18-3L-VL11-PEP variants is performed with Epibase IV platform (Algonomics/LONZA). Algonomics Epibase IV™ evaluates immunogenicity potential of antibody therapeutics by directly measuring T-cell responses in a naive donor population representative. It identifies T-cell epitopes on proteins and allows direct comparison of immunogenicity profiles of protein leads. Combined with the Algonomics Epibase™ in silico tool, Algonomics Epibase IV™ cellular assays facilitate the selection of best leads. Characterization and comparison of T-cell responses raised by de-immunized VL18-3L-VL11-DI #3-PEP DI and VL18-3L-VL11-DI #5-PEP DI variants is performed in a fifty (50) donor population using DC: CD4 assays. Evaluation of VL18-3L-VL11 fused to wild-type and de-immunized PEP are also performed and used as control.

1. An agent comprising a therapeutic molecule linked to at least one albumin-binding domain, or an albumin-binding fragment or derivative thereof,
wherein said albumin-binding domain comprises an amino acid sequence corresponding to SEQ ID NO:1 (PEP), wherein said domain is modified by at least one amino acid substitution selected from the group consisting of El2D, T29H-K35D, and A45D, said substitutions referring to amino acid positions in SEQ ID NO:1; and
wherein said agent has an increased serum half life as compared to the therapeutic molecule.

2. The agent of claim 1 wherein said albumin-binding domain comprises an amino acid sequence corresponding to SEQ ID NO:31.

3-5. (canceled)

6. The agent of claim 1 wherein the serum half life is increased by at least about 5 fold, by at least about 8 fold, or by at least about 10 fold compared to the therapeutic molecule; and/or
wherein the serum half life is at least about 30 hours or at least about 40 hours; and/or
wherein said agent also has increased solubility compared to the therapeutic molecule.

7-11. (canceled)

12. The agent of claim 6 wherein the solubility is increased by at least about 2 fold, by at least about 5 fold, by at least about 10 fold, or by at least about 15 fold.

13-15. (canceled)

16. The agent of claim 1 wherein the linkage is via a linker.

17. The agent of claim 16 wherein the linker is a peptide linker or a peptide linker comprising an amino acid sequence corresponding to SEQ ID NO:30

18. (canceled)

19. The agent of claim 1 wherein the therapeutic molecule is a therapeutic polypeptide or peptide.

20. The agent of claim 19 wherein said therapeutic polypeptide or peptide is linked to said albumin-binding domain as a fusion.

21. The agent of claim 1 wherein the therapeutic molecule is selected from the group consisting of protamine, gp60, gp30, gpl8, protein A, a G protein, a protein transduction domain, a toxin, a cytotoxin, a radionuclie, and a macrocyclic chelator.

22-23. (canceled)

24. The agent of claim 1 wherein the therapeutic molecule comprises a dimer of two antibody single domains or antigen-binding fragments thereof, wherein said domains comprise light chain variable domains.

25-27. (canceled)

28. The agent according to claim 25 wherein said agent antagonizes binding of human TNF-alpha to a TNF-alpha receptor and/or
wherein said agent cross-reacts with at least one non-primate mammalian TNF-alpha; and/or
wherein said agent cross-reacts with a TNF-alpha of at least two non-human mammals, said at least two other mammals being a rodent and a non-rodent species.

29-30. (canceled)

31. The agent according to claim 24 wherein said dimer is further de-immunized by eliminating at least one T_{H} epitope in at least one of said variable domains.

32. The agent according to claim 31 wherein said at least one variable domain comprises an amino acid sequence corresponding to SEQ ID NO:2 (VL18), which is also de-immunized by at least one amino acid substitution selected from the group consisting of T7Q, V15P, (A51V-L54R/A51V-L54E), K63S, E79K, (C805), T91A, and L111K, said substitutions referring to amino acid positions in SEQ ID NO:2^{.} and/or
wherein said at least one variable domain comprises an amino acid sequence corresponding to SEQ ID NO:3 (VL11), which is also de-immunized by at least one amino acid substitution selected from the group consisting of T7Q, V15P, R31 S, (A51V-54R /A51V-L54E), K63S, E79K, (C805), T91A, A100S, and E106K, said substitutions referring to amino acid positions in SEQ ID NO:3; and/or
wherein said dimer comprises at least one amino acid sequence selected from the group consisting of SEQ ID NOs:20-24, SEQ ID NOs: 25-29, SEQ ID NOs: 34-44 (VL18-3L-VL11/PEP variants), and a TNF-alpha-binding fragment or derivative thereof.

33-34. (canceled)

35. A method of enhancing the efficacy of a therapeutic molecule in a subject, comprising:
providing an agent comprising said therapeutic molecule linked at least one albumin-binding domain, or an albumin-binding fragment or derivative thereof,
wherein said albumin-binding domain comprises an amino acid sequence corresponding to SEQ ID NO:1 (PEP) wherein said domain is modified by at least one amino acid substitution selected from the group consisting of E12D, T29H-K35D, and A45D, said substitutions referring to amino acid positions in SEQ ID NO:1; and
administering said agent to said subject, wherein said agent has an increased serum half life as compared to the therapeutic molecule.

36-39. (canceled)

40. The method of claim 35 wherein the linkage is via a linker, the linkage is via a peptide linker, or the linkage is via a fusion.

41. (canceled)

42. The method of claim 35 wherein the therapeutic molecule is a therapeutic polypeptide or peptide; and/or
wherein the therapeutic molecule is selected from the group consisting of protamine, gp60, gp30, gp18, protein A, a G protein, a protein transduction domain, a toxin, a cytotoxin, a radionuclide, and a macrocyclic chelator; and/or
wherein the therapeutic molecule is selected from the group consisting of an antibody, and antibody fragment, a monoclonal antibody, multispecific antibody, humanized antibody, synthetic antibody, chimeric antibody, polyclonal antibody, single-chain Fv (scFv), single chain antibody, anti-idiotypic (anti-Id) antibody, diabody, minibody, nanobody, single domain antibody, Fab fragment, F(ab′) fragment, disulfide-linked bispecific Fv (sdFv), and intrabody; and/or
wherein the therapeutic molecule comprises a dimer of two antibody single domains or antigen-binding fragments thereof, wherein said domains comprise light chain variable domains.

43-47. (canceled)

48. A pharmaceutical composition comprising the agent according to claim 1 and a pharmaceutically acceptable carrier.

49. A nucleic acid comprising a nucleotide sequence encoding the agent according to claim 19,
a vector comprising the nucleic acid, or
a host cell comprising the vector.

50-51. (canceled)

52. A method of making the agent according to claim 19 comprising:
(i) providing a host cell comprising a vector encoding said agent;
(ii) culturing said cell under conditions allowing expression of said agent; and
(iii) recovering said agent from said culture.

53. The agent of claim 21 wherein said therapeutic molecule is selected from the group consisting of an antibody, an antibody fragment, a monoclonal antibody, multispecific antibody, humanized antibody, synthetic antibody, chimeric antibody, polyclonal antibody, single-chain Fv (scFv), single chain antibody, anti-idiotypic (anti-Id) antibody, diabody, minibody, nanobody, single domain antibody, Fab fragment, F(ab′) fragment, disulfide-linked bispecific Fv (sdFv), and intrabody.
